SEQUENCE LISTING (53 sequences)

SEQ ID NO: 1 - protein, 22 aa, synthetic construct
EESKDAGIRTLVMLDEQGEQLD

SEQ ID NO: 2 - protein, 22 aa, synthetic construct
IESQDAGIKTITMLDEQKEQLN

SEQ ID NO: 3 - protein, 16 aa, synthetic construct
  CROSSLNK 5..12: note = Macrocyclic crosslink is formed by substituting the non-natural amino acids R-octenylalanine and S-pentenylalanine
  MOD_RES 5: note = R-octenylalanine
  MOD_RES 12: note = S-pentenylalanine
SKDAAIRGLVMADEQC

SEQ ID NO: 4 - protein, 19 aa, synthetic construct
  CROSSLNK 4..11: note = Macrocyclic crosslink is formed by substituting the non-natural amino acids R-octenylalanine and S-pentenylalanine
  MOD_RES 4: note = R-octenylalanine
  MOD_RES 11: note = S-pentenylalanine
SKDAGIRTLVALDEQGEQL

SEQ ID NO: 5 - protein, 21 aa, synthetic construct
  MOD_RES 4: note = S-pentenylalanine
  MOD_RES 8: note = S-pentenylalanine
  MOD_RES 15: note = S-pentenylalanine
  MOD_RES 19: note = S-pentenylalanine
  CROSSLNK 4..8: note = Macrocyclic crosslink is formed by substituting the non-natural amino acids S-pentenylalanine
  CROSSLNK 15..19: note = Macrocyclic crosslink is formed by substituting the non-natural amino acids S-pentenylalanine
SKDAGIRALVMLDEAGEQADR

SEQ ID NO: 6 - protein, 19 aa, synthetic construct
  CROSSLNK 5..9: note = Macrocyclic crosslink is formed by substituting the non-natural amino acids S-pentenylalanine
  CROSSLNK 12..16: note = Macrocyclic crosslink is formed by substituting the non-natural amino acids S-pentenylalanine
  MOD_RES 5: note = S-pentenylalanine
  MOD_RES 9: note = S-pentenylalanine
  MOD_RES 12: note = S-pentenylalanine
  MOD_RES 16: note = S-pentenylalanine
SKDAAIRTAVMADEQAEQL

SEQ ID NO: 7 - protein, 21 aa, synthetic construct
  CROSSLNK 5..9: note = Macrocyclic crosslink is formed by substituting the non-natural amino acids S-pentenylalanine
  CROSSLNK 15..19: note = Macrocyclic crosslink is formed by substituting the non-natural amino acids S-pentenylalanine
  MOD_RES 5: note = S-pentenylalanine
  MOD_RES 9: note = S-pentenylalanine
  MOD_RES 15: note = S-pentenylalanine
  MOD_RES 19: note = S-pentenylalanine
SKDAAIRTAVMLDEAGEQADR

SEQ ID NO: 8 - protein, 18 aa, synthetic construct
  CROSSLNK 3..7: note = Macrocyclic crosslink is formed by substituting the non-natural amino acids S-pentenylalanine
  CROSSLNK 10..14: note = Macrocyclic crosslink is formed by substituting the non-natural amino acids S-pentenylalanine
  MOD_RES 3: note = S-pentenylalanine
  MOD_RES 7: note = S-pentenylalanine
  MOD_RES 10: note = S-pentenylalanine
  MOD_RES 14: note = S-pentenylalanine
EEAKDAAIRALVMADEQC

SEQ ID NO: 9 - protein, 19 aa, synthetic construct
  CROSSLNK 4..8: note = Macrocyclic crosslink is formed by substituting the non-natural amino acids S-pentenylalanine
  CROSSLNK 11..15: note = Macrocyclic crosslink is formed by substituting the non-natural amino acids S-pentenylalanine
  MOD_RES 4: note = S-pentenylalanine
  MOD_RES 8: note = S-pentenylalanine
  MOD_RES 11: note = S-pentenylalanine
  MOD_RES 15: note = S-pentenylalanine
SKDAGIRALVALDEAGEQL

SEQ ID NO: 10 - protein, 17 aa, synthetic construct
EESKDAGIRTLVMLDEQ

SEQ ID NO: 11 - protein, 19 aa, synthetic construct
  MOD_RES 5: note = R-octenylalanine
  MOD_RES 12: note = S-pentenylalanine
  CROSSLNK 5..12: note = Macrocyclic crosslink is formed by substituting the non-natural amino acids R-octenylalanine and S-pentenylalanine
SKDAAIRTLVMADEQGEQL

SEQ ID NO: 12 - protein, 19 aa, synthetic construct
  CROSSLNK 8..15: note = Macrocyclic crosslink is formed by substituting the non-natural amino acids R-octenylalanine and S-pentenylalanine
  MOD_RES 8: note = R-octenylalanine
  MOD_RES 15: note = S-pentenylalanine
SKDAGIRALVMGDEAGEQL

SEQ ID NO: 13 - protein, 19 aa, synthetic construct
  CROSSLNK 9..16: note = Macrocyclic crosslink is formed by substituting the non-natural amino acids R-octenylalanine and S-pentenylalanine
  MOD_RES 9: note = R-octenylalanine
  MOD_RES 16: note = S-pentenylalanine
SKDAGIRTAVMGDEQAEQL

SEQ ID NO: 14 - protein, 19 aa, synthetic construct
  CROSSLNK 5..9: note = Macrocyclic crosslink is formed by substituting the non-natural amino acids S-pentenylalanine
  CROSSLNK 11..15: note = Macrocyclic crosslink is formed by substituting the non-natural amino acids S-pentenylalanine
  MOD_RES 5: note = S-pentenylalanine
  MOD_RES 9: note = S-pentenylalanine
  MOD_RES 11: note = S-pentenylalanine
  MOD_RES 15: note = S-pentenylalanine
SKDAAIRTAVALDEAGEQL

SEQ ID NO: 15 - protein, 16 aa, synthetic construct
  MOD_RES 4: note = R-octenylalanine
  MOD_RES 11: note = S-pentenylalanine
  CROSSLNK 4..11: note = Macrocyclic crosslink is formed by substituting the non-natural amino acids R-octenylalanine and S-pentenylalanine
SKDAGIRTLVALDEQC

SEQ ID NO: 16 - protein, 16 aa, synthetic construct
RQIKIWFQNRRMKWKK

SEQ ID NO: 17 - protein, 19 aa, synthetic construct
VKRGLKLRHVRPRVTRMDV

SEQ ID NO: 18 - protein, 27 aa, synthetic construct
GALFLGFLGAAGSTMGAWSQPKKKRKV

SEQ ID NO: 19 - protein, 21 aa, synthetic construct
KETWWETWWTEWSQPKKKRKV

SEQ ID NO: 20 - protein, 18 aa, synthetic construct
LLIILRRRIRKQAHAHSK

SEQ ID NO: 21 - protein, 22 aa, synthetic construct
MVRRFLVTLRIRRACGPPRVRV

SEQ ID NO: 22 - protein, 28 aa, synthetic construct
MVKSKIGSWILVLFVAMWSDVGLCKKRP

SEQ ID NO: 23 - protein, 18 aa, synthetic construct
KLALKLALKALKAALKLA

SEQ ID NO: 24 - protein, 27 aa, synthetic construct
GWTLNSAGYLLGKINLKALAALAKKIL

SEQ ID NO: 25 - protein, 28 aa, synthetic construct
LSTAADMQGVVTDGMASGLDKDYLKPDD

SEQ ID NO: 26 - protein, 26 aa, synthetic construct
DPKGDPKGVTVTVTVTVTGKGDPKPD

SEQ ID NO: 27 - protein, 24 aa, synthetic construct
RRIRPRPPRLPRPRPRPLPFPRPG

SEQ ID NO: 28 - protein, 17 aa, synthetic construct
CSIPPEVKFNKPFVYLI

SEQ ID NO: 29 - protein, 6 aa, synthetic construct
PFVYLI

SEQ ID NO: 30 - protein, 15 aa, synthetic construct
SDLWEMMMVSLACQY

SEQ ID NO: 31 - protein, 18 aa, synthetic construct
KLALKLALKALKAALKLA

SEQ ID NO: 32 - protein, 12 aa, synthetic construct
PLILLRLLRGQF

SEQ ID NO: 33 - protein, 12 aa, synthetic construct
PLIYLRLLRGQF

SEQ ID NO: 34 - protein, 15 aa, synthetic construct
KLWMRWYSPTTRRYG

SEQ ID NO: 35 - protein, 22 aa, synthetic construct
MGLGHLLVLAAALQGAKKKRKV

SEQ ID NO: 36 - protein, 17 aa, synthetic construct
  MOD_RES 3: note = S-pentenylalanine
  MOD_RES 7: note = S-pentenylalanine
  MOD_RES 10: note = S-pentenylalanine
  MOD_RES 14: note = S-pentenylalanine
  CROSSLNK 3..7: note = Macrocyclic crosslink is formed by substituting the non-natural amino acids S-pentenylalanine
  CROSSLNK 10..14: note = Macrocyclic crosslink is formed by substituting the non-natural amino acids S-pentenylalanine
EEAKDAAIRALVMADEQ

SEQ ID NO: 37 - protein, 17 aa, synthetic construct
  CROSSLNK 3..7: note = Macrocyclic crosslink is formed by substituting the non-natural amino acids S-pentenylalanine
  CROSSLNK 10..14: note = Macrocyclic crosslink is formed by substituting the non-natural amino acids S-pentenylalanine
  MOD_RES 3: note = S-pentenylalanine
  MOD_RES 7: note = S-pentenylalanine
  MOD_RES 10: note = S-pentenylalanine
  MOD_RES 14: note = S-pentenylalanine
EEAKDAAIRALVMADEQ

SEQ ID NO: 38 - protein, 34 aa, synthetic construct
  MOD_RES 20: note = S-pentenylalanine
  MOD_RES 24: note = S-pentenylalanine
  MOD_RES 27: note = S-pentenylalanine
  MOD_RES 31: note = S-pentenylalanine
  CROSSLNK 20..24: note = Macrocyclic crosslink is formed by substituting the non-natural amino acids S-pentenylalanine
  CROSSLNK 27..31: note = Macrocyclic crosslink is formed by substituting the non-natural amino acids S-pentenylalanine
IRQIKIWFQNRRMKWKKEEAKDAAIRALVMADEQ

SEQ ID NO: 39 - protein, 33 aa, synthetic construct
RQIKIWFQNRRMKWKKEESKDAGIRTLVMLDEQ

SEQ ID NO: 40 - protein, 30 aa, synthetic construct
  CROSSLNK 16..20: note = Macrocyclic crosslink is formed by substituting the non-natural amino acids S-pentenylalanine
  CROSSLNK 23..27: note = Macrocyclic crosslink is formed by substituting the non-natural amino acids S-pentenylalanine
  MOD_RES 16: note = S-pentenylalanine
  MOD_RES 20: note = S-pentenylalanine
  MOD_RES 23: note = S-pentenylalanine
  MOD_RES 27: note = S-pentenylalanine
GRKKRRQRRRPPQEEAKDAAIRALVMADEQ

SEQ ID NO: 41 - protein, 30 aa, synthetic construct
GRKKRRQRRRPPQEESKDAGIRTLVMLDEQ

SEQ ID NO: 42 - protein, 26 aa, synthetic construct
LVESQGEMIDRIEYNVEHAVDYVERA

SEQ ID NO: 43 - protein, 26 aa, synthetic construct
LVENQGEMLDNIELNVMHTVDHVEKA

SEQ ID NO: 44 - protein, 15 aa, synthetic construct
IIGNLRHMALDMGNE

SEQ ID NO: 45 - protein, 15 aa, synthetic construct
ILGNLKDMALNIGNE

SEQ ID NO: 46 - protein, 140 aa, synthetic construct
EEQEKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIAQWHTL

SEQ ID NO: 47 - protein, 140 aa, synthetic construct
EEPEKLGDICTSLRYVPTAGKLTVCILEAKNLKKMDVGGLSDPYVKIHLMQNGKRLKKKKTTVKKKTLNPYFNESFSFEIPFEQIQKVQVVVTVLDYDKLGKNEAIGKIFVGSNATGTELRHWSDMLANPRRPIAQWHSL

SEQ ID NO: 48 - protein, 13 aa, synthetic construct
GRKKRRQRRRPPQ

SEQ ID NO: 49 - protein, 151 aa, synthetic construct
EKLGDICTSLRYVPTAGKLTVCILEAKNLKKMDVGGLSDPYVKIHLMQNGKRLKKKKTTVKKKTLNPYFNESFSFEIPFEQIQKVQVVVTVLDYDKLGKNEAIGKIFVGSNATGTELRHWSDMLANPRRPIAQWHSLKPEEEVDALLGKNK

SEQ ID NO: 50 - protein, 151 aa, synthetic construct
EKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIAQWHTLQVEEEVDAMLAVKK

SEQ ID NO: 51 - protein, 151 aa, synthetic construct
EKLGDICFSLAYVPTAGKLTVVILAAKNLKKMDVGGLSDPYVKIHLMQNGKRLKKKKTTIKKNTLNPAYNESFSFEVPFEQIQKVQVVVTVLDYDKIGKNDAIGKVFVGYNSTGAELRHWSDMLANPAAPIAQWHTLQVEEEVDAMLAVKK

SEQ ID NO: 52 - protein, 22 aa, synthetic construct
EESKDAGIRTLVMLDEQGEQLD

SEQ ID NO: 53 - protein, 19 aa, synthetic construct
  CROSSLNK 5..9: note = Macrocyclic crosslink is formed by substituting the non-natural amino acids S-pentenylalanine
  CROSSLNK 12..16: note = Macrocyclic crosslink is formed by substituting the non-natural amino acids S-pentenylalanine
  MOD_RES 5: note = S-pentenylalanine
  MOD_RES 9: note = S-pentenylalanine
  MOD_RES 12: note = S-pentenylalanine
  MOD_RES 16: note = S-pentenylalanine
SKDAAIRTAVMADEQAEQL

(19) United States
(12) Patent Application Publication
(10) Pub. No.: US 2025/0163122 A1
(43) Pub. Date: May 22, 2025

(54) COMPOSITIONS AND METHODS FOR TREATING AIRWAY MUCUS DYSFUNCTION

(71) Applicants: BOARD OF REGENTS, THE UNIVERSITY OF TEXAS SYSTEM, Austin, TX (US); THE BOARD OF TRUSTEES OF THE LELAND STANFORD JUNIOR UNIVERSITY, Standford, CA (US); UNIVERSITAET ULM, Ulm (DE)

(72) Inventors: Burton F. DICKEY, Houston, TX (US); Michael J. TUVIM, Houston, TX (US); Philip JONES, Houston, TX (US); Axel T. BRUNGER, San Francisco, CA (US); Ying LAI, Chengdu (CN); Manfred FRICK, Pfaffenhofen a.d. Roth (DE)

(21) Appl. No.: 18/838,956

(22) PCT Filed: Feb. 16, 2023
(86) PCT No.: PCT/US2023/062758

§ 371 Date: Aug. 15, 2024

Related U.S. Application Data
(60) Provisional application No. 63/311,001, filed on Feb. 16, 2022.

(51) Int. Cl. C07K 14/705; A61K 9/00; A61K 38/17; A61P 11/12; C12N 15/113

(52) U.S. Cl. CPC C07K 14/705; A61K 9/0073; A61K 38/177; A61P 11/12; C12N 15/113; C07K 2319/10

(57) ABSTRACT

Polypeptide constructs comprising a first peptide attached to a cell penetrating peptide, wherein the first peptide has both homology to a portion of the SNARE protein SNAP-25 and non-natural amino acids comprising one or two macrocyclic crosslinks, are provided herein. These polypeptide constructs are useful for disrupting the primary interface between SNAP-25 or its homolog and Syt1 or its homolog. Methods for treating a subject with mucus hypersecretion-based airway obstruction and/or developed mucus occlusions are also described, as well as methods of inhibiting mucin secretion in an airway epithelial cell.

### CROSS-REFERENCES TO RELATED APPLICATIONS

This application claims priority to and the benefit of U.S. Provisional Patent Application Ser. No. 63/311,001, filed Feb. 16, 2022, the contents of which is incorporated herein in its entirety by this reference as if fully set forth herein.

### STATEMENT AS TO RIGHTS TO INVENTIONS MADE UNDER FEDERALLY SPONSORED RESEARCH AND DEVELOPMENT

This invention was made with Government support under grant numbers R01 HL129795, RO1 A1137319, and MH063105 awarded by the National Institutes of Health. The Government has certain rights in the invention.

### REFERENCE TO A SEQUENCE LISTING SUBMITTED AS A XML FILE

This application is accompanied by a sequence listing entitled .xml, created February ______, 2023, which is approximately ______ bytes in size. This sequence listing is incorporated herein by reference in its entirety. This sequence listing is submitted herewith via EFS-Web, and is in compliance with 37 C.F.R. § 1.824(a)(2)-(6) and (b).

### BACKGROUND

Airway mucus plaques contribute to the pathogenesis of common lung diseases, including asthma, COPD, cystic fibrosis, and bronchiectasis. Plaques form because of increased mucin production with rapid mucin secretion (together, “mucin hypersecretion”) or inadequate surface liquid. Both result in the formation of mucus that is excessively concentrated, viscoelastic and adhesive.

Mucins are high molecular weight, highly glycosylated proteins that are packaged into secretory granules and secreted from airway epithelial cells. Mucin secretion occurs at both a low baseline rate and a high stimulated rate. Stimulated mucin secretion involves interactions between the Ca²⁺ sensitive synaptotagmin-2 (Syt2) protein with components of the SNARE (soluble NSF attachment protein receptor) complex. The SNARE complex is involved in packaging and delivering material to various parts of a cell via vesicles. In particular, there is a primary interface where the SNARE protein SNAP-23 binds to the C2B domain of Syt2 that is important for stimulated mucin secretion.

Available therapeutic drugs address mucin production and surface liquid production that occur when airway mucus plaques are formed, but do not target the stimulated hypersecretion of mucin mediated by the interaction between Syt2 and the SNAP-23.

### BRIEF SUMMARY

Various examples are described for compositions and methods for treating mucus dysfunction.

Provided herein are polypeptide constructs comprising a first peptide comprising a portion of the SNARE protein SNAP-25A (a close homolog of SNAP-23) or a variant thereof attached to a cell penetrating peptide, wherein the first peptide is a hydrocarbon-stapled peptide comprising non-natural amino acids connected via at least one macrocyclic crosslink. In some embodiments, the polypeptide constructs are useful for specifically inhibiting mucin hypersecretion. In some embodiments, the polypeptide constructs are useful for specifically disrupting other Ca2+-triggered membrane fusion processes, such synaptic neurotransmitter release. In some embodiments, the cell penetrating domain is penetratin.

Also provided herein are various Syt2 expression inhibitors. Syt2 expression inhibitors include Syt2 targeting polynucleotides or polynucleotide complexes, such shRNAs, ASOs, siRNAs, or miRNAs, and one or more gene editing system components (e.g., at least one of a targeted nuclease or a guide RNA).

Also provided are pharmaceutical compositions comprising such a polypeptide construct and a pharmaceutically acceptable excipient. In addition, provided are pharmaceutical compositions comprising such a Syt2 expression inhibitor. Also provided are kits and devices containing such pharmaceutical compositions.

Also provided are methods for treating a subject with mucus hypersecretion-based airway obstruction and/or developed mucus occlusions. In some instances, the methods can include administering a therapeutically effective amount of a polypeptide construct as described herein to a subject in need thereof. The polypeptide constructs can be administered in conjunction with an inhibitor of at least one of Munc18, VAMP8, Munc13, or Stx3. These are homologs of neuronal synaptic proteins and are implicated in basal and/or stimulated mucin secretion in airway cells. In some instances, the methods can include administering a therapeutically effective amount of a Syt2 expression inhibitor as described herein to a subject in need thereof.

Also provided are methods of inhibiting mucin secretion in an airway epithelial cell and methods of inhibiting Syt2-mediated stimulated mucin secretion, triggered by Ca²⁺ release after ATP or methacholine bind to helpta-helical plasma membrane receptors coupled to Gq. In some instances, the methods include contacting a cell with an effective amount of a polypeptide construct as described herein. In some instances, the methods include contacting a cell with an effective amount of a Syt2 expression inhibitor as described herein.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF DRAWINGS

The accompanying drawings, which are incorporated into and constitute a part of this specification, illustrate one or more certain examples and, together with the description of the example, serve to explain the principles and implementations of the certain examples.

FIG. 1A shows an analysis of sections of bronchial airways of mice were stained with PAFS to indicate mucin with fluorescence according to certain aspects of this disclosure. In naïve mice without airway inflammation, scant intracellular mucin is visible (top row, left). Treatment with IL-13 increases mucin synthesis, resulting in abundant intracellular mucin (top row, right, visible as more obvious protrusions from the surface). Subsequent treatment with ATP induces mucin secretion, reducing intracellular mucin in wild type (C57Bl/6J) and Syt2^{F/F} mice (data not shown). Scale bar=50 μm.

FIG. 1B shows a measurement of fractional mucin secretion by analysis of images of airways of wild type, Syt2^{F/F}, and Syt2^{D/D} mice treated with IL-13 compared to those of mice treated with IL-13 followed by ATP according to certain aspects of this disclosure. Shown are individual data points and box plots: the whiskers show the min and max values (excluding outliers), the box limits are the 25th and 75th percentiles, and the center line denotes the median. n=2 independent experiments combined to give a total of 9-11 mice per group. Comparison to the group of F/F mice is by Student's t-test, ** p<0.01.

FIG. 1C shows an analysis of transverse sections of bronchial airways of mice treated with IL-13 to increase mucin synthesis, then with methacholine (Mch) to induce smooth muscle contraction and mucin secretion, fixed with methacarn, and stained with PAFS to demonstrate lumenal mucus accumulation and residual intracellular mucin according to certain aspects of this disclosure. Scale bar=50 μm.

In FIG. 1D, the sum of lumenal mucus cross-sectional area in the left lung measured at 500 μm intervals is shown for wild type, Syt2^{F/F}, and Syt2^{D/D} mice according to certain aspects of this disclosure. Shown are individual data points and box plots as defined in the description for FIG. 1B. n=2 independent experiments combined to give a total of 10-11 mice per group. Comparison to the group of F/F mice is by Student's t-test, * p<0.05.

FIG. 2A shows the percentage of α-helical content in the stapled peptides, estimated by dividing the mean residue ellipticity [φ]222obs by the reported [φ]222obs for a model helical decapeptide according to certain aspects of this disclosure.

FIG. 2B shows the interactions between Cy3-labeled SP9 or P0 and unlabeled Syt1 C2B, Syt1 C2B_QM, and Syt2_C2B as measured by bulk fluorescence anisotropy (see Example 1) according to certain aspects of this disclosure. Data points are shown as mean±SEM along with individual data points from n=3-7 independent experiments. Hill equations were fit to estimate the dissociation constant Kd, where the Hill coefficients were constrained to one.

FIG. 2C shows the end points of five independent 1-μsec simulations of SP9:Syt1-C2B (top) and P9:Syt1-C2B (bottom) according to certain aspects of this disclosure. The simulations started from a conformation that was derived from the crystal structure with PDB ID 5W5C (data not shown; also see EXTENDED DATA FIGS. 2H and I of U.S. Provisional Patent Application Ser. No. 63/311,001). For one simulation of P9:Syt1 C2B, the P9 peptide dissociated around 168 nanosecondsdata not shown; also see EXTENDED DATA FIG. 2J of U.S. Provisional Patent Application Ser. No. 63/311,001.

FIGS. 3A-3G show the effect of stapled peptides on fusion with complete airway system reconstitution according to certain aspects of this disclosure. FIG. 3A shows the domain structure of Munc13-2 and its fragment (Munc13-2*). FIG. 3B shows a diagram of a single vesicle content mixing assay with complete reconstitution. PM: vesicles with reconstituted Stx3 and SNAP-23 that mimic the plasma membrane of epithelial cells; SM: vesicles with reconstituted Stx3/Munc18-2 complex; SG: mucin-containing secretory granules. 0.5 μM Munc13-2* fragment was added after formation of the SM vesicles and during all subsequent stages. 10 μM stapled peptide was added together with SG vesicles and was present during all subsequent stages. FIG. 3C shows the effect of SP9 on vesicle association. FIG. 3D shows the average probabilities of Ca²⁺-independent fusion events per second. FIG. 3E shows the corresponding Ca²⁺-triggered fusion amplitudes of the first 1-see time bin upon 500 μM Ca²⁺-injection. FIG. 3F shows the cumulative Ca²⁺-triggered fusion probability within 1 min. FIG. 3G shows the decay rate (1/τ) of the Ca²⁺-triggered fusion histogram. The fusion probabilities and amplitudes were normalized to the number of analyzed SG—airway PM vesicle pairs (see TABLE 4). FIGS. 3C, 3E, 3F, and 3G show means±SEM for multiple independent repeat experiments (see TABLE 2). FIG. 3G shows decay constants and error estimates computed from the covariance matrix upon fitting the corresponding histograms with a single exponential decay function using the Levenberg-Marquardt algorithm. * p<0.05, ** p<0.01 by Student's t-test, compared to the experiment without the stapled peptides.

FIGS. 4A-4E show the proved stapled peptides inhibit mucin secretion from airway epithelial cells according to certain aspects of this disclosure. Human airway epithelial cells (HAECs) were treated with SP9-Cy3 or SP9-Cy3 conjugated to CPPs or biotin. Biotin-SP9-Cy3 was bound to streptavidin-conjugated C2 or CRM197.

FIG. 4A shows confocal collapse (projected) images of fixed HAECs treated with SP9-Cy3 or SP9-Cy3 conjugated to bacterial toxins (C2, CRM197) or CPPs. Scale bar=10 μm.

FIG. 4B shows quantitative analysis of intracellular Cy3 fluorescence for each peptide in Muc5ac+ HAECs. Data are mean±SEM. n=30-59 cells for each condition. *** p<0.001 assessed by one-way ANOVA followed by post-hoc Dunnett's test.

FIG. 4C shows representative western blots for Muc5ac on apical surface of untreated HAECs (control), HAECs treated with 10 μM of SP9-Cy3, or HAECs treated with 10 μM of either SP9-Cy3 or P9-Cy3 conjugated to CPPs for 30 min before stimulation. basl. represents Muc5ac secretion during a 30 min period before stimulation (baseline) and exp. represents Muc5ac secreted within a 30 min experimental period with (IL-13+ATP) or without (IL-13) stimulation of HAECs with 10 μM ATP. Cells were treated with IL-13 to induce mucous metaplasia.

FIG. 4D shows bar graphs indicating the ratio of baseline/reference wash secretion (fold increase of baseline secretion over reference secretion) for each condition in FIG. 4C. Shown are individual data points and box plots as defined below for FIG. 4E. n=1-2 independent experiments from 4 independent donors for each condition.

FIG. 4E shows bar graphs indicating the ratio of experimental/baseline (fold increase of stimulated secretion over baseline secretion) for each condition in FIG. 4C. Shown are individual data points and box plots: the whiskers show the min and max values (excluding outliers), the box limits are the 25% and 75% percentiles, and the center line denotes the median. n=1-2 independent experiments from 4 donors for each condition. * p<0.05 assessed by two-way ANOVA followed by post-hoc Dunnett's test.

FIGS. 5A-5E show that stapled peptide PEN-SP9-Cy3 inhibits mucin secretion and airway mucus occlusion in mice according to certain aspects of this disclosure.

FIG. 5A shows transverse sections of bronchial airways of mice fixed 30 min after aerosol delivery of PBS or Cy3 (red fluorescent) labeled peptides showing intracellular uptake of peptides. Bright fluorescent staining shows nuclei labeled with DAPI. Fluorescent staining is overlaid on differential interference contrast images. Scale bar=25 μm. Left to right, samples are PBS, PE-P9-Cy3, and PEN-SP9-Cy3.

FIG. 5B shows averaged uptake fractions in box plots. They were generated by counting fluorescence indicating labeled peptide uptake as the numerator and fluorescent nuclei as the denominator, with a total of 260 cells counted in 6 sections from 3 mice for PEN-SP9-Cy3 and 361 cells in 6 sections from 3 mice for PEN-SP9-Cy3. Points are the mean of individual slides, whiskers show the min and max values (excluding outliers), the box limits are the 25th and 75th percentiles, and the center line denotes the median. Comparison of the two groups by Student's t-test shows p>0.05. Left to right, samples are PBS, PE-P9-Cy3, and PEN-SP9-Cy3.

FIG. 5C shows fractional mucin secretion measured by analysis of images of airways of mice treated with IL-13 and comparing those to mice treated with IL-13 followed by methacholine. Box plots show individual data points for each mouse, and the remainder of the plots are as in FIG. 5B. n=2 independent experiments combined to give a total of 8-10 mice per group. Comparison of mice pretreated with peptides to the group of mice pretreated with PBS is by Student's t-test, *** p<0.001. Left to right, samples are PBS, PE-P9-Cy3, and PEN-SP9-Cy3.

FIG. 5D shows airway lumenal mucus accumulation measured by image analysis as in FIG. 1, except that right lungs were examined instead of left lungs. Micrographs are transverse sections of small airways, so the epithelial cells are smaller than in c. Scale bar=25 μm. Left to right, samples are PBS, PE-P9-Cy3, and PEN-SP9-Cy3.

FIG. 5E shows the sum of lumenal mucus cross-sectional area in the caudal lobe of the right lung measured at 500 μm intervals is indicated for the same mouse genotypes as in FIG. 5E. Box plots show individual data points for each mouse, and the remainder of the plots are as in FIG. 5B. n=2 independent experiments combined to give a total of 7-13 mice per group. Comparison of mice pretreated with peptides to the group of mice pretreated with PBS is by Student's t-test, * p<0.05. Left to right, samples are PBS, PE-P9-Cy3, and PEN-SP9-Cy3.

FIG. 6 shows size exclusion chromatography (SEC) profiles of stapled peptides. Each peptide was loaded into a Superdex 75 column. The dashed line indicates the border of the void volume at ˜8 ml.

FIGS. 7A and 7B show screening data for the effect of the provided stapled peptides in an ensemble lipid mixing assay for neuronal SNAREs and Syt1 according to certain aspects of this disclosure. FIG. 7A shows the effect of stapled peptides in Ca²⁺-independent ensemble lipid mixing for neuronal SNAREs and Syt1. The two groups of vesicles were mixed at the same molar ratio with a final lipid concentration of 0.1 mM. Shown are time traces of FRET efficiency upon mixing neuronal PM- and SV-vesicles. Relative initial rates were also measured (data not shown; also see also see EXTENDED DATA FIG. 4F of U.S. Provisional Patent Application Ser. No. 63/311,001). FIG. 7B shows the effect of stapled peptides in Ca²⁺-triggered ensembled lipid mixing in neuronal system. The two groups of vesicles were mixed at the same molar ratio with a final lipid concentration of 0.1 mM. Shown are time traces of FRET efficiency upon mixing neuronal PM- and SV-vesicles. Relative initial rates were also measured (data not shown; also see also see EXTENDED DATA FIG. 4H of U.S. Provisional Patent Application Ser. No. 63/311,001).

FIGS. 8A-8E show the effect of the provided stapled peptides in a single vesicle content mixing assay for neuronal SNAREs and Syt1 or its “quintuple” mutant Syt1_QM according to certain aspects of this disclosure. Shown are the results of a single vesicle content mixing assay with a plasma membrane mimic vesicles with reconstituted Stx1A and SNAP-25A, and a synaptic vesicle mimic with reconstituted VAMP2 and Syt1. After SV—neuronal PM vesicle association, vesicle pairs either undergo Ca²⁺-independent fusion or remain associated until fusion is triggered by Ca²⁺ addition. 10 μM of each specific stapled peptide was added together with SV vesicles and was present in all subsequent stages. FIG. 8A shows the effects of stapled peptides on vesicle association. The corresponding Ca²⁺-independent fusion probabilities were also measured (data not shown; also see EXTENDED DATA FIG. 5C of U.S. Provisional Patent Application Ser. No. 63/311,001). FIG. 8B shows the corresponding average probabilities of Ca²⁺-independent fusion events per second. The corresponding Ca²⁺-triggered fusion probabilities were also measured (data not shown; also see EXTENDED DATA FIG. SE of U.S. Provisional Patent Application Ser. No. 63/311,001). FIG. 8C shows the corresponding Ca²⁺-triggered fusion amplitudes of the first 1-see time bin upon 500 μM Ca²⁺-injection. FIG. 8D shows the cumulative Ca²⁺-triggered fusion probability within 1 min. FIG. 8E shows the corresponding decay rate (1/τ) of the Ca²⁺-triggered fusion histogram. FIGS. 8A-8E show means±SEM for multiple independent repeat experiments. FIG. 8A is vesicle association; FIG. 8B is Ca²⁺-independent fusion (per second); FIG. 8C is Ca²⁺-triggered fusion amplitude; FIG. 8D is Ca²⁺-triggered fusion (per min); and FIG. 8E is Ca²⁺-triggered fusion synchronization. Decay constants and error estimates in FIG. 8E were computed from the covariance matrix upon fitting the corresponding histograms with a single exponential decay function using the Levenberg-Marquardt algorithm. In FIGS. 8B, 8C, and 8D, * p<0.05, ** p<0.01 by Student's t-test, compared to the experiment without the stapled peptides.

FIGS. 9A-9C show data relating to the preparation of airway PM and SG vesicles according to certain aspects of this disclosure. FIG. 9A shows SDS-PAGE analysis of airway PM and SG vesicles with reconstituted airway SNAREs and Syt2. For details of the reconstitution and lipid compositions, see Example 1. FIG. 9B shows cryo-EM images of airway PM and SG proteoliposomes. Scale bar=100 nm. FIG. 9C shows diameter distributions for airway PM and SG vesicles.

FIGS. 10A-10E show that the provided stapled peptides inhibit Ca²⁺-triggered vesicle fusion with reconstituted airway epithelial SNAREs and Syt2 according to certain aspects of this disclosure. FIG. 10A shows the effects of stapled peptides on vesicle association. FIG. 10B shows the average probabilities of Ca²⁺-independent fusion events per second. The corresponding Ca²⁺-triggered fusion probabilities were also measured (data not shown; also see EXTENDED DATA FIG. 7E of U.S. Provisional Patent Application Ser. No. 63/311,001). FIG. 10C shows the corresponding Ca²⁺-triggered fusion amplitudes of the first 1-sec time bin upon 500 μM Ca²⁺-injection (F). FIG. 10D shows the cumulative Ca²⁺-triggered fusion probability within 1 min. FIG. 10E shows the decay rate (1/i) of the Ca²⁺-triggered fusion histogram. The effects of 10 μM of each of the specified peptides on vesicle association using the assay described above, the corresponding Ca²⁺-independent fusion probabilities, and the corresponding average probabilities of Ca²⁺-independent fusion events per second were also measured (data not shown; also see EXTENDED DATA FIGS. 7I-7K of U.S. Provisional Patent Application Ser. No. 63/311,001). For FIGS. 10A-10D, the means±SEM for multiple independent repeat experiments are shown. Decay constants and error estimates in FIG. 10E was computed from the covariance matrix upon fitting the corresponding histograms with a single exponential decay function using the Levenberg-Marquardt algorithm. * p<0.05, ** p<0.01 by Student's t-test, compared to the experiment without the stapled peptides.

FIGS. 11A and 111B show that the provided stapled peptides penetrate epithelial cells when conjugated to CPPs and inhibit mucin secretion from airway epithelium cells according to certain aspects of this disclosure. FIG. 11A shows a representative western blot for Muc5ac on apical surface of untreated HAECs (control 1 and 2) or HAECs treated with 100 μM SP9-Cy3, PEN-SP9-Cy3, TAT-SP9-Cy3, PEN-P9-Cy3, or TAT-P9-Cy3 for 24 h before stimulation. Wash represents Muc5ac accumulated during culture and before start of experiment. Baseline represents unstimulated Muc5ac secretion during a 15 min period after removal of accumulated Muc5ac and experimental represents Muc5ac secreted within 15 min of stimulation with (ATP) or without (no ATP) 100 μM ATP. Lysate represents MUC5AC within HAECs at the end of the experiment. Cells were treated with IL-13 to induce mucous metaplasia. FIG. 11B is bar graphs showing the ratio of experimental/baseline secretion (fold increase of stimulated secretion over baseline secretion) following 24 h pre-incubation with 100 NM of the respective peptides. Shown are individual data points and box plots: the whiskers show the min and max values (excluding outliers), the box limits are the 25% and 75% percentile and the center line denotes the median. * p<0.05 assessed by two-way ANOVA followed by post-hoc Dunnett's test. For each condition, data is shown from left to right for control, SP9-Cy3, PEN-SP9-Cy3, TAT-SP9-Cye, PEN-p9-Cy3, and TAT-P9-Cy3.

FIGS. 12A-12C show that cytoplasmic delivery of SP9 depends on endocytosis and endosomal escape. FIG. 12A shows a schematic of SP9 endocytosis and edosomal escape and indicates the steps inhibited by dynasore and chloroquine. FIGS. 12B and 12C show the effects of adding 80 μM dynasore or 100 μM chloroquine on the mean fluorescence intensity of Cy3 intracellular fluorescence after incubation of single CALU-3 cells with 100 μM PEN-SP9-Cy3 conjugates (N>1200 cells/condition in 3 independent experiments). Statistical analysis: One-way ANOVA, *** denotes p<0.001).

### DETAILED DESCRIPTION

Provided herein are compositions and methods for treating obstruction of airways due to mucin hypersecretion by airway epithelial cells, which contributes to the pathogenesis of a number of diseases, including asthma, COPD, cystic fibrosis, and bronchiectasis. Mucin hypersecretion includes elevated mucin production and stimulated mucin secretion. The invention includes compositions that disrupt stimulated mucin secretion and methods of using such compositions. In some instances, the compositions are polypeptide constructs that disrupt the interaction between soluble NSF attachment protein receptor (SNARE) vesicle fusion proteins and Ca²⁺ sensor proteins involved in stimulated mucin secretion. In some instances, the compositions are polynucleotides or polynucleotide complexes that result in reduced expression of Syt2 in an airway epithelial cell, such as an airway epithelial cell.

Secretion of proteins such as mucin relies on vesicle-mediated transport, which in turn relies on lipid bilayer fusion. Fusion proteins, such as SNARE proteins, play an essential role in secretion, supplying the energy to overcome the high kinetic barrier to mediate lipid bilayer fusion and mediating fusion of vesicles with a target membrane efficiently and specifically. Since membrane fusion is a key process within all living cells, SNARE proteins are highly conserved among different organisms and in different vesicle-mediated processes with a single organism.

The SNARE protein family consists of at least 60 membrane-associated proteins in mammalian cells. Among the best studied SNARE proteins are those involved in the targeting of synaptic vesicles, which carry neurotransmitters, in neurons (Gerald, K., 2002). SNARE proteins can be functionally distinguished as v-SNAREs, which are located on vesicles, and t-SNAREs which are located on the target membrane, which associate together to form the SNARE protein complex.

The underlying molecular mechanisms for membrane fusion are superficially similar for both class 1 viral fusion proteins and SNARE-mediated fusion, wherein formation of helical bundles drives membranes together, leading to membrane fusion. However, for stimulated mucin secretion, SNARE proteins must cooperate with other factors, in particular, the Ca²⁺ sensor synaptotagmin. Synaptotagmins are a family of proteins containing an N-terminal transmembrane region, a linker, and two C-terminal domains, called C2A and C2B. The C2 domain of a subset of synaptotagmins binds to Ca²⁺ (Mackler et al., 2002), an interacts with other SNARE components. For example, between 2 and 3 Ca²⁻ ions bind to the C2B domain of synaptotagmin-1 (Syt1). Id.

The primary interface between the neuronal SNARE complex and the C2B domain of Syt1 is a specific interface that is conserved in all species and across the other fast isoforms for neurotransmitter release, synaptotagmin-2 (Syt2) and synaptotagmin-9 (Zhou et al., 2015). Many of the key residues involved in the primary interface are located in the t-SNARE SNAP-25A. Residues involved in the primary interface are critical for Ca²⁺-triggered fusion in a reconstituted system and in neuronal cultures (Zhou et al., 2015; Zhou et al., 2017).

Primary sequence conservation suggests that the primary interface also exists in other systems that utilize SNAREs and synaptotagmins, such as stimulated exocytosis in neurons, endocrine cells, and exocrine cells (Zhou et al., 2015). In particular, mucin exocytosis in airway secretory cells is mediated by SNAREs, Syt2, and other factors (Jaramillo et al., 2018; Davis and Dickey, 2008; Fahy et al., 2010). Syt2 is selectively expressed in airway secretory cells compared to ciliated cells, and it serves as a critical sensor for stimulated but not baseline mucin secretion (Tuvim et al., 2009). Syntaxin-3 (Stx3) and SNAP-23 are also highly expressed in airway epithelial cells (Riento et al., 1998; Ren et al. 2015). In stimulated mucin secretion, Ca²⁺ is released from the endoplasmic reticulum (ER) via the activated inositol triphosphate (IP3) receptor. IP3 is generated by phospholipase C (PLC) upon binding of agonists (such as ATP or methacholine) to hepta-helical receptors in the plasma membrane coupled to Gq. The released Ca²⁺ in turn binds to Syt2 on the granule vesicle and then triggers SNARE-mediated fusion of the granule with the plasma membrane, leading to mucin secretion (Jaramillo et al., 2018; Davis & Dickey, 2008; Tuvim et al., 2009).

Both baseline and stimulated mucin secretion are impaired in SNAP-23 heterozygous mutant mice (Ren et al., 2015), VAMP8 knock-out mice (Jones et al., 2012), and Munc13-2 knock-out mice (Zhu et al., 2008). In contrast, stimulated mucin secretion is selectively impaired in Munc18-2 mutant mice (Kim et al., 2012; Jaramillo et al., 2019). Stx3 binds and colocalizes with Munc18-2 (Riento et al., 1998) and overexpression of Munc18-2 reduces the level of Stx3/SNAP-23 binary complex (Kim et al., 2012). These finding suggested that, similar to neurotransmitter release, SNAREs (Stx3, SNAP-23, and VAMP8), Syt2, Munc13-2, and Munc18-2 are among the key components that drive membrane fusion between mucin-containing granules and the plasma membrane (Jaramillo et al., 2018; Davis & Dickey, 2008; Fahy et al., 2010).

The invention described in this disclosure is based in part on the discovery that hydrocarbon-stapled peptides can disrupts Ca²⁺-triggered membrane fusion by interfering with the primary interface between the neuronal SNARE complex and synaptotagmin-1. In reconstituted systems with these neuronal synaptic proteins or with their airway homologues syntaxin-3, SNAP-23, VAMP8, synaptotagmin-2, along with Munc13-2 and Munc18-2, SP9 strongly suppressed Ca²⁺-triggered fusion at physiological Ca²⁺ concentrations. Conjugation of cell penetrating peptides to SP9 resulted in efficient delivery into cultured human airway epithelial cells and mouse airway epithelium, where it markedly reduced stimulated mucin secretion in both systems, and substantially attenuated mucus occlusion of mouse airways. Based on this work, peptides that disrupt Ca²⁺-triggered membrane fusion may allow therapeutic modulation of mucin secretory pathways.

### I. Definitions

The terms “peptide,” “polypeptide,” and “protein” are used interchangeably herein to refer to a polymer of amino acid residues linked by covalent peptide bonds. All three terms apply to naturally occurring amino acid polymers and non-natural amino acid polymers, as well as to amino acid polymers in which one (or more) amino acid residue is an artificial chemical mimetic of a corresponding naturally occurring amino acid. Unless otherwise specified, the terms encompass amino acid chains of any length, including full-length proteins.

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

As used herein, a “chemically modified amino acid” refers to an amino acid whose side chain has been chemically modified. For example, a side chain can be modified to comprise a new functional group, such as an olefin, a thiol, a carboxylic acid, or an amino group. A side chain can also be modified to comprise a signaling moiety, such as a fluorophore or a radiolabel. Post-translationally modified amino acids are also included in the definition of chemically modified amino acids.

Also contemplated are “conservative amino acid substitutions.” By way of example, a conservative amino acid substitution can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

The terms “nucleic acid,” “nucleotide,” and “polynucleotide” refer to deoxyribonucleic acids (DNA) or ribonucleic acids (RNA) and polymers. The term includes, but is not limited to, single-, double-, or multi-stranded DNA or RNA, genomic DNA, cDNA, and DNA-RNA hybrids, as well as other polymers comprising purine and/or pyrimidine bases or other natural, chemically modified, biochemically modified, non-natural, synthetic, or derivatized nucleotide bases. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), orthologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

“Percentage of sequence identity” is determined by comparing two optimally aligned sequences over a comparison window, wherein the portion of the sequence (e.g., a peptide of the invention) in the comparison window may comprise additions or deletions (i.e., gaps) as compared to the reference sequence that does not comprise additions or deletions, for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

“Identical” and “identity,” in the context of two or more nucleic acids or polypeptide sequences, refer to two or more sequences or subsequences that are the same. Sequences are “substantially identical” to each other if they have a specified percentage of nucleotides or amino acid residues that are the same (e.g., at least 75%, at least 80%, at least 85%, at least 90%, at least 95%, or at least 99% identical over a specified region), when compared and aligned for maximum correspondence over a comparison window, or designated region as measured using one of the following sequence comparison algorithms or by manual alignment and visual inspection.

“Similarity” and “percent similarity,” in the context of two or more polypeptide sequences, refer to two or more sequences or subsequences that have a specified percentage of amino acid residues that are either the same or similar as defined by a conservative amino acid substitutions (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% similar over a specified region), when compared and aligned for maximum correspondence over a comparison window, or designated region as measured using one of the following sequence comparison algorithms or by manual alignment and visual inspection. Sequences are “substantially similar” to each other if, for example, they are at least 20%, at least 25%, at least 30%, at least 35%, at least 40%, at least 45%, at least 50%, or at least 55% similar to each other.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA, 90: 5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test sequence to the reference sequence is less than about 0.2, more preferably less than about 0.01, and most preferably less than about 0.001.

When only two polypeptides are being compared, one sequence acts as the reference polypeptide sequence, the homologous regions are aligned, and the identical amino acids are counted and recorded as a percent of the total reference polypeptide sequence, which is the “percent identity.”

“Hydrocarbon-stapled peptides” are peptides whose structure has been stabilized or altered by, for example, incorporating at least one all-hydrocarbon macrocyclic crosslink.

A “macrocyclic crosslink” is a covalent bond formed when pairs of amino acids bearing two terminal alkenes, such as α,α-disubstituted non-natural amino acids bearing olefin tethers of various lengths, undergo ring closing metathesis. See, e.g., Schafmeister, C. E., et al., J. Am. Chem. Soc. 122(24): 5891-5892 (2000).

As used herein, the term “cell penetration peptide” refers to an amino acid sequence that, when linked to a second peptide, causes or enhances the ability of the second peptide to cross membranes of a cell when the cell is contacted by the cell penetration peptide linked to the second peptide.

An “airway epithelial cell” includes any one of a variety of cell types found in the respiratory epithelium including, for example, basal cells, club cells, ciliated cells, goblet cells, tuft cells, pulmonary ionocytes, pulmonary neuroendocrine cells, hillock cells, and microfold cells. An airway secretory cell is an airway epithelial cell that is variously termed a club, Clara, and goblet cell. While these cell types are of the same lineage, their appearance can change depending on the degree of mucin production.

As used herein, “treating” or “treatment” of any disease or disorder refers to preventing or ameliorating a disease or disorder in a subject or a symptom thereof. The term ameliorating refers to any therapeutically beneficial result in the treatment of a disease state, e.g., COPD or cystic fibrosis, lessening in the severity or progression, or curing thereof. Treating or treatment also encompass prophylactic treatments that reduce the incidence of a disease or disorder in a subject and/or reduce the incidence or reduce severity of a symptom thereof. Thus, treating or treatment includes ameliorating at least one physical parameter or symptom. Treating or treatment includes modulating the disease or disorder, either physically (e.g., stabilization of a discernible symptom) or physiologically (e.g., stabilization of a physical parameter) or both. Treating or treatment includes delaying, preventing increases in, or decreasing mucin hypersecretion. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating airway obstruction in a subject by administering a fusion protein or modified protein as described in this disclosure is considered to be a treatment if there is at least a 10% reduction in one or more symptoms of airway obstruction in a subject as compared to a control. Thus, the reduction can be a 10%, 20%, 30%, 40%, 50%, 60%, 70%, 80%, 90%, 100%, or any percent reduction in between 10% and 100% as compared to native or control levels. In some embodiments, formulations comprising a fusion protein or modified protein as described herein are administered to the subject until the subject exhibits amelioration of at least one symptom of airway obstruction and/or is demonstrated to have a sustained decrease in mucin secretion, e.g., as measured by immunoassay. In some instances, the formulation is administered to the subject until mucin hypersecretion is undetectable, i.e. below the level of detection, such that only basal mucin secretion can be detected after IL-13 and ATP treatment by the assay methodology employed. In some instances, the subject exhibits undetectable mucin hypersecretion 1-4 weeks, 2-4 weeks, 2-12 weeks, 4-12 weeks, or 12-24 weeks after last administration of the formulation. It is understood that treatment does not necessarily refer to a cure or complete ablation of the disease, condition, or symptoms of the disease or condition.

As used herein, the term “therapeutically effective amount” refers to an amount of polypeptide construct as provided herein that, when administered to a subject, is effective to achieve an intended purpose, e.g., to reduce airway obstruction, to reduce or ameliorate at least one symptom of an airway obstruction disease (e.g., COPD, cystic fibrosis), and/or otherwise reduce the length of time that a patient experiences a symptom of an airway obstruction disease, or extend the length of time before a symptom may recur. The term therapeutically effective amount may be referred to herein as effective amount, with the context depending on the subject who is receiving treatment or in reference to in vitro effects. An effective amount is also one in which any toxic or detrimental effects of the composition are outweighed by the therapeutically beneficial effects. In some instances, an effective amount is not a dosage so large as to cause adverse side effects, such as excessive coughing, increased propensity for pulmonary infections, and the like. An effective amount may vary with the subject's age, condition, and sex, the extent of the disease in the subject, frequency of treatment, the nature of concurrent therapy (if any), the method of administration, and the nature and scope of the desired effect(s) (Nies et ah, Chapter 3 In: Goodman & Gilman's The Pharmacological Basis of Therapeutics, 9th Ed., Hardman et ah, eds., McGraw-Hill, New York, NY, 1996). and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics or treatments that are administered to the subject. Although individual needs may vary, determination of optimal ranges for effective amounts of formulations is within the skill of the art. It should also be understood that a specific dosage and treatment regimen for any particular subject also depends upon the judgment of the treating medical practitioner (e.g., doctor or nurse).

As used herein, the term “subject” refers to animals such as mammals, including, but not limited to, primates (e.g., humans), cows, sheep, goats, horses, dogs, cats, rabbits, rats, mice and the like.

As used herein, the term “pharmaceutically acceptable excipient” refers to a substance that aids the administration of an active agent to a subject. By “pharmaceutically acceptable,” it is meant that the excipient is compatible with the other ingredients of the formulation and is not deleterious to the recipient thereof. Pharmaceutical excipients useful in the compositions include, but are not limited to, binders, fillers, disintegrants, lubricants, glidants, coatings, sweeteners, flavors and colors.

As used herein, the terms “about” and “around” indicate a close range around a numerical value when used to modify that specific value. If “X” were the value, for example, “about X” or “around X” would indicate a value from 0.9X to 1.1X, e.g., a value from 0.95X to 1.05X, or a value from 0.98X to 1.02X, or a value from 0.99X to 1.01X. Any reference to “about X” or “around X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

### II. Peptide Constructs

Provided herein are polypeptides comprising a hydrocarbon-stapled peptide attached to a cell penetrating peptide. Also provided herein are polypeptides comprising a hydrocarbon-stapled peptide attached to a non-biological material. The hydrocarbon-stapled peptide is derived from a portion of SNAP-25A that forms an α-helix in the full-length SNARE complex that is a part of the primary interface with synaptotagmin. The hydrocarbon-stapled peptide, also referred to as the first peptide in this disclosure, contains one or two macrocyclic crosslinks that stabilize the formation of an α-helical peptide conformation.

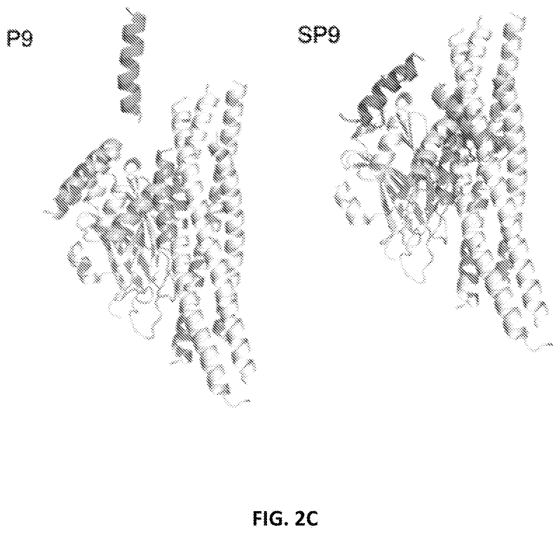

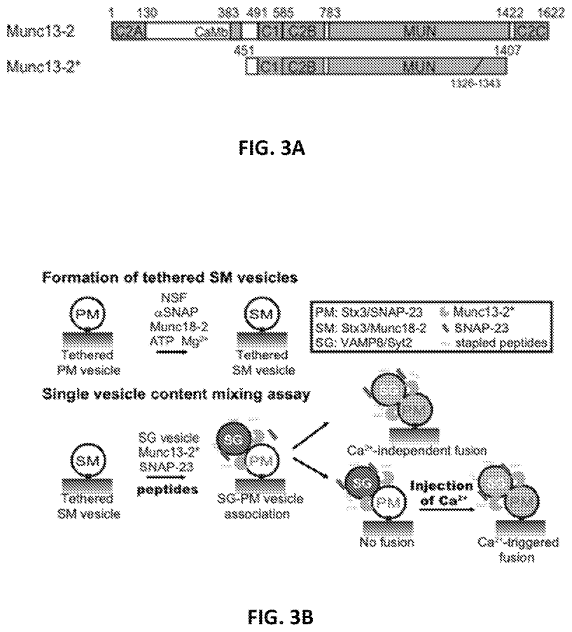

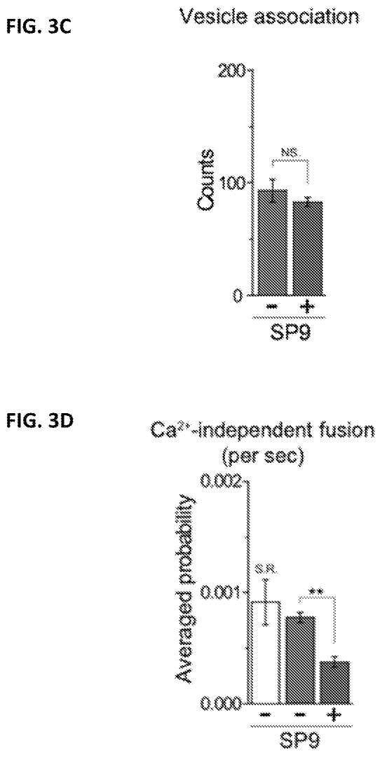

In some embodiments, the first peptide comprises one side that contains amino acid residues that interact with amino acid residues in a synaptotagmin protein. In the context of this disclosure, this side of the first peptide is the primary interface-facing side of the first peptide. In some embodiments, the first peptide also comprises one side that does not contain amino acids that interact with amino acid residues in a synaptotagmin protein. In the context of this disclosure, this side of the first peptide is the non-binding side of the first peptide. As discussed above, the primary interface of SNARE proteins with Syt2 and other synaptotagmin homologs is conserved across species. The crystal structure of the primary interface between the SNARE complex (VAMP-2), Stx1, SNAP-25A, and the C2B domain of Syt1 indicates which amino acid residues in the first peptide (e.g., SP9) likely face the primary interface (data not shown; also see EXTENDED DATA FIGS. 2B and 2C of U.S. Provisional Patent Application Ser. No. 63/311,001).

Primary sequence alignments between neuronal and airway systems (Stx1A vs Stx3, SNAP-25A vs SNAP-23, and Syt1 vs Syt2) indicate that the residues directly involved in interactions at the primary interface are identical for Syt1/Syt2 (see SEQ ID NO: 46, amino acid positions 268-407; SEQ ID NO:47, amino acid positions 269-408) and mostly identical for SNAP-25A/SNAP-23 (see SEQ ID NOs: 1-2 (amino acid positions 37-58) and SEQ ID NOs: 44-45 (amino acid positions 156-170); also see EXTENDED DATA FIGS. 2A and 2B of U.S. Provisional Patent Application Ser. No. 63/311,001). For example, residues that are important for the primary interface include R281, E295, Y338, R398, and R399 in Syt1 C2B (also corresponding to residues mutated in Syt1_QM); K40, D51, E52, E55, Q56, and D166 in SNAP-25A; and D231, E234, and E238 in Stx1A (see SEQ ID NOS: 42-47; also see EXTENDED DATA FIGS. 2A and 2B of U.S. Provisional Patent Application Ser. No. 63/311,001). These are among the highly conserved amino acid residues when comparing SEQ ID NO: 42 (Stx1A aa 222-247) to SEQ ID NO: 43 (Stx3 aa 222-247), when comparing SEQ ID NO: 44 (SNAP-25 aa 156-170) to SEQ ID NO: 45 (SNAP-23 aa 156-170), and when comparing SEQ ID NO: 46 (Syt1, aa 268-407) to SEQ ID NO: 47 (Syt2, aa 268-407) (also see EXTENDED DATA FIGS. 2A and 2B of U.S. Provisional Patent Application Ser. No. 63/311,001).

In some embodiments, the macrocyclic crosslinks are located on the first peptide so as to not interfere with the interaction of the first peptide with the region of a synaptotagmin homolog that forms part of the primary interface and binds to components of the SNARE complex. In some embodiments, the macrocyclic crosslink is located between non-natural amino acid pairs that are on the non-binding side of the first peptide. Said another way, in some embodiments, the macrocyclic crosslinks of the first peptide are positioned such that they oriented away from the primary interface-facing side of the first peptide.

In some embodiments, the first peptide comprises SEQ ID NO: 1 or a sequence having at least 64% identity (e.g., at least 64%, 65%, 70%, 75%, 80%, 85%, 90% 95%, or 99% identity) thereto. In some embodiments, the first peptide comprises SEQ ID NO: 2, or a sequence having at least 64% identity (e.g., at least 64%, 65%, 70%, 75%, 80%, 85%, 90% 95%, or 99% identity) thereto.

SEQ ID NO: 1 and SEQ ID NO: 2 correspond to residues 37-58 of human SNAP-25A and residues 32-53 of human SNAP-23, respectively. Other proteins within this family of SNARE proteins are identifiable by amino acid sequence homology using, for example, the BLAST algorithm. Since they may perform analogous functions in vivo, the corresponding peptide sequences from such sequence homologs may functionally substitute for SEQ ID NO: 1 and/or SEQ ID NO: 2.

In some embodiments, the first peptide comprises SEQ ID NO: 1 or SEQ ID NO: 2 or a portion or variant thereof (e.g., having at least 64% identity (e.g., at least 64%, 65%, 70%, 75%, 80%, 85%, 90% 95%, or 99% identity) thereto) and one or more pairs of non-natural amino acids in which each pair forms a macrocyclic crosslink. In some embodiments, the first peptide comprises a variant of SEQ ID NO: 1 or SEQ ID NO: 2 comprising a non-native amino acid residue in at least one amino acid position not predicted to interact with Syt1 C2B based on the crystal structure of the SP9-Syt2 C2B complex. In some embodiments, the first peptide comprises a variant of SEQ ID NO: 1 or SEQ ID NO: 2 with a non-native amino acid residue in a position corresponding to at least one of amino acid positions 2, 6, 9, 13, 17, or 18 of the SP9 sequence as set forth in SEQ ID NO:8. In some embodiments, the variant of SEQ ID NO: 1 or SEQ ID NO: 2 comprises a non-native amino acid residue at least one of positions 2, 6, 9, 13, 17, or 18 of SEQ ID NO: 8.

In some embodiments, the first peptide comprises one pair of non-natural amino acids that form a macrocyclic crosslink, and the non-natural amino acids flank six contiguous amino acid residues in the first peptide. In some embodiments, the one pair of non-natural amino acids corresponds to amino acids 6 and 13, 7 and 14, 10 and 17, or 11 and 18 of SEQ ID NO: 1 or SEQ ID NO: 2. In some embodiments, the first peptide comprises SEQ ID NO: 3 or 4.

In some embodiments, the first peptide comprises two pairs of non-natural amino acids that each form a macrocyclic crosslink, and each pair of non-natural amino acids flanks three continguous amino acids. In some embodiments, the first pair of non-natural amino acids correspond to amino acids 3 and 7, 6 and 10, or 7 and 11 in SEQ ID NO: 1 or SEQ ID NO: 2, and the second pair of non-natural amino acids correspond to amino acids 10 and 14, 13 and 17, 14 and 18, or 17 and 21 in SEQ ID NO: 1 or SEQ ID NO: 2. In some embodiments, the first peptide comprises SEQ ID NO: 5, 6, 7, 8, or 9.

In some embodiments, the polypeptide constructs comprise one or more cell penetration peptides linked to the first peptide (e.g., to the N-terminus of the first peptide or to the C-terminus of the first peptide). Cell penetration peptides are positively charged and 5-30 amino acids long, and characterized by their ability to penetrate into biological membranes, often taking their attached cargo with them. A number of cell penetration peptides can be linked to the first peptide so as to enhance delivery of the first peptide to target cells in vitro and/or in vivo (see, e.g., Guidotti 2017 and Derakhshankhah & Jafari 2018). The polypeptide construct can have the first peptide and one or more cell penetration peptides in any orientation. In some embodiments, the C-terminus of the first peptide is linked to the N-terminus of the cell penetration peptide. In some embodiments, the N-terminus of the first peptide is linked to the C-terminus of a first cell penetration peptide(s) and C-terminus of the first peptide is linked to the N-terminus of a second cell penetration peptide(s). In some embodiments, the cell penetration peptide is a cationic peptide having 5-25 total amino acid residues and at least 5 arginine residues, lysine residues, or a combination thereof. In some embodiments, the cell penetration peptide is a polyarginine ranging in length from 5 residues to 25 residues. In some embodiments, the cell penetration peptide comprises an amino acid sequence set forth in Table 1 or a sequence having at least 70% identity (e.g., at least 70%, 75%, 80%, 85%, 90%, 95%, or 99% identity) thereto. In some embodiments, the cell penetrating peptide is selected so that the polypeptide cannot stimulate IL-13-dependent release of mucin without stimulation by ATP or methacholine. IL-13 is the cytokine interleukin 13.

Table 1 shows stapled peptide sequences according to various embodiments of this disclosure. Shown are the staple types and location of linkages involved in macrocyclic crosslinks within each peptide. A single macrocyclic crosslink is typically formed by substituting the non-natural amino acid S-pentylalanine at i, i+4 positions, resulting in a three amino acid gap flanking the macrocyclic crosslink. Two macrocyclic crosslinks within a single polypeptide are typically formed by substituting R-octenylalanine (R8)/S-pentenylalanine (S5) or S-octenylalanine (S8)/R-pentenylalanine (R5) at i, i+7 positions. Also indicated are any N- or C-terminal modifications such as acetyl (Ac) or amide groups.

TABLE 1
Stapled peptide sequences and their N- and C-terminal modifications.

| SEQ | | | | | | | |
|---|---|---|---|---|---|---|---|
| ID | | Staple | | N- | C- | No. of | |
| NO | Peptide | Type | Linkages | term | term | residues | Peptide Sequence |
| 1 | P0 | none | | | | 22 | EESKDAGIRTLVMLDEQGEQLD |
| 10 | P9 | none | | | | 17 | EESKDAGIRTLVMLDEQ |
| 5 | SP1 | i, i + 4 | [(4, 8; | Ac | amide | 21 | Ac-SKD(S5)GIR(S5)LVMLDE(S5)GEQ |
| | | | 15, 19)] | | | | (S5)DR-amide |
| 11 | SP2 | i, i + 7 | [(5, 12)] | Ac | amide | 19 | Ac-SKDA(R8)IRTLVM(S5)DEQGEQL-amide |
| 3 | SP3 | i, i + 7 | [(5, 12)] | Ac | amide | 16 | Ac-SKDA(R8)IRGL VM(S5)DEQC-amide |
| 6 | SP4 | i, i + 4 | [(5, 9; | Ac | amide | 19 | Ac-SKDA(S5)IRT(S5)VM(S5)DEQ(S5) |
| | | | 12, 16)] | | | | EQL-amide |
| 12 | SP5 | i, i + 7 | [(8, 15)] | Ac | amide | 19 | Ac-SKDAGIR(R8)LVMGDE(S5)GEQL-amide |
| 13 | SP6 | i, i + 7 | [(9, 16)] | Ac | amide | 19 | Ac-SKDAGIRT(R8)VMGDEQ(S5)EQL-amide |
| 7 | SP7 | i, i + 4 | [(5, 9; | Ac | amide | 21 | Ac-SKDA(S5)IRT(S5)VMLDE(S5)GEQ |
| | | | 15, 19)] | | | | (S5)DR-amide |
| 4 | SP8 | i, i + 7 | [(4, 11)] | Ac | amide | 19 | Ac-SKD(R8)GIRTLV(S5)LDEQGEQL-amide |
| 8 | SP9 | i, i + 4 | [(3, 7; | Ac | amide | 18 | Ac-EE(S5)KDA(S5)IR(S5)LVM(S5)DEQC- |
| | | | 10, 14)] | | | | amide |
| 9 | SP10 | i, i + 4 | [(4, 8; | Ac | amide | 19 | Ac-SKD(S5)GIR(S5)LV(S5)LDE(S5) |
| | | | 11, 15)] | | | | GEQL-amide |
| 14 | SP11 | i, i + 4 | [(5, 9; | Ac | amide | 19 | Ac-SKDA(S5)IRT(S5)V(S5)LDE(S5) |
| | | | 11, 15)] | | | | GEQL-amide |
| 15 | SP12 | i, i + 7 | [(4, 11)] | Ac | amide | 16 | Ac-SKD(R8)GIRTLV(S5)LDEQC |

TABLE 2
Cell Penetration Peptides

| SEQ ID | | |
|---|---|---|
| NO: | Sequence | Origin/Name |
| 16 | RQIKIWFQNRRMKWKK | Penetratin |
| 17 | VKRGLKLRHVRPRVTRMDV | Chemically synthesized |
| 18 | GALFLGFLGAAGSTMGAWSQPKKKRKV | HIV glycoprotein 41/SV40 T antigen |
| | | NLS |
| 19 | KETWWETWWTEWSQPKKKRKV | Tryptophan-rich cluster/SV40 T |
| | | antigen NLS |
| 20 | LLIILRRRIRKQAHAHSK | Vascular endothelial cadherin |
| 21 | MVRRFLVTLRIRRACGPPRVRV | p14ARF protein |
| 22 | MVKSKIGSWILVLFVAMWSDVGLCKKRP | N terminus of unprocessed bovine |
| | | prion protein |
| 23 | KLALKLALKALKAALKLA | Chemically synthesized |
| 24 | GWTLNSAGYLLGKINLKALAALAKKIL | Chimeric galanin-mastoparan |
| 25 | LSTAADMQGVVTDGMASGLDKDYLKPDD | Azurin |
| 26 | DPKGDPKGVTVTVTVTVTGKGDPKPD | Synthetic |
| 27 | RRIRPRPPRLPRPRPRPLPFPRPG | Bactenecin family of antimicrobial |
| | | peptides |
| 28 | CSIPPEVKFNKPFVYLI | α1-Antitrypsin |
| 29 | PFVYLI | Derived from synthetic C105Y |
| 30 | SDLWEMMMVSLACQY | CHL8 peptide phage clone |
| 31 | KLALKLALKALKAALKLA | MAP |
| 32 | PLILLRLLRGQF | Pept1 |
| 33 | PLIYLRLLRGQF | Pept2 |
| 34 | KLWMRWYSPTTRRYG | IVV-14 |
| 35 | MGLGHLLVLAAALQGAKKKRKV | Ig(v) |
| 48 | GRKKRRQRRRPPQ | TAT |

In some embodiments, the polypeptide constructs comprise the first peptide linked to a non-biological material. In some embodiments, the non-biological material is a small molecular compound, a metal chelate, a polymer, a hydrogel, or a nanoparticle. See, e.g., Shu, J. Y. et al., Peptide-polymer conjugates: from fundamental science to application. Annu. Rev. Phys. Chem. 64, 631-657 (2013) and Xiao, Y. et al., Diabetic wound regeneration using peptide-modified hydrogels to target re-epithelialization. Proc. Natl. Acad. Sci. USA. 113(40), E5792-E5801 (2016). Nanoparricles (NPs) have shown their potential to serve as conjugate scaffolds that not only improve the functionality of peptides but also implement abiotic characteristics, often resulting in synergistic effects. See, e.g., Jeong, Wj., et al. Peptide-nanoparticle conjugates: a next generation of diagnostic and therapeutic platforms?Nano Convergence 5, 38 (2018).

The polypeptide constructs as described herein may be synthesized by solid-phase peptide synthesis methods, during which N-α-protected amino acids having protected side chains are added stepwise to a growing polypeptide chain linked by its C-terminus to a solid support, e.g., polystyrene beads. Various chemistries, resins, protecting groups, protected amino acids and reagents can be employed as described, for example, by Barany and Merrifield, “Solid-Phase Peptide Synthesis,” in The Peptides: Analysis, Synthesis, Biology Gross and Meienhofer (eds.), Academic Press, N.Y., vol. 2, pp. 3-284 (1980); Atherton et al., SolidPhase Peptide Synthesis: A Practical Approach, IRL Press (1989); Bodanszky, Peptide Chemistry, A Practical Textbook, 2nd Ed., Springer-Verlag (1993)); and Chan et al. Fmoc Solid Phase Peptide Synthesis: A Practical Approach, Oxford University Press (2000).

Non-limiting examples of support materials for solid-phase peptide synthesis include polystyrene (e.g., microporous polystyrene resin, mesoporous polystyrene resin, macroporous polystyrene resin; including commercially-available Wang resins, Rink amide resins, and trityl resins), glass, polysaccharides (e.g., cellulose, agarose), polyacrylamide resins, polyethylene glycol, or copolymer resins (e.g., comprising polyethylene glycol, polystyrene, etc.). The solid support may have any suitable form factor. For example, the solid support can be in the form of beads, particles, fibers, or in any other suitable form factor. Non-limiting examples of protecting groups (e.g., N-terminal protecting groups) include Fmoc, Boc, allyloxycarbonyl (Alloc), benzyloxycarbonyl (Z), and photolabile protecting groups. Sidechain protecting groups include, but are not limited to, Fmoc; Boc; cyclohexyloxycarbonyl (Hoc); allyloxycarbonyl (Alloc); mesityl-2-sulfonyl (Mts); 4-(N-methylamino)butanoyl (Nmbu); 2,4-dimethylpent-3-yloxycarbonyl (Doc); 1-(4,4-dimethyl-2,6-dioxocyclohex-1-ylidene)-3-ethyl (Dde); 1-(4,4-dimethyl-2,6-dioxocyclohex-1-ylidene)-3-methylbutyl (ivDde); 4-methyltrityl (Mtt). Additional protecting groups and methods for their addition and removal from supported peptides are described, for example, by Isidro-Llobet et al. Chem. Rev. 2009, 19: 2455-2504.

A base may be used to activate or complete the activation of amino acids prior to exposing the amino acids to immobilized peptides. In some embodiments, the base is a non-nucleophilic bases, such as triisopropylethylamine, N,N-diisopropylethylamine, certain tertiary amines, or collidine, that are non-reactive to or react slowly with protected peptides to remove protecting groups. A coupling agent may be used to form a bond with the C-terminus of an amino acid to facilitate the coupling reaction and the formation of an amide bond. The coupling agent may be used to form activated amino acids prior to exposing the amino acids to immobilized peptides. Any suitable coupling agent may be used. In some embodiments, the coupling agent is a carbodiimide, a guanidinium salt, a phosphonium salt, or a uronium salt. Examples of carbodiimides include, but are not limited to, N,N′-dicyclohexylcarbodiimide (DCC), 1-ethyl-3-(3-dimethylaminopropyl)carbodiimide (EDC), and the like. Examples of phosphonium salts include, but are not limited to, such as (benzotriazol-1-yloxy)tripyrrolidinophosphonium hexafluorophosphate (PyBOP); bromotris (dimethylamino) phosphonium hexafluorophosphate (BroP); and the like. Examples of guanidinium/uronium salts include, but are not limited to, O-(benzotriazol-1-yl)-N,N,N′,N′-tetramethyluronium hexafluorophosphate (HBTU); 2-(7-aza-1H-benzotriazole-1-yl)-1,1,3,3-tetramethyluronium hexafluorophosphate (HATU); 1-[(1-(cyano-2-ethoxy-2-oxoethylideneaminooxy) dimethylaminomorpholino)]uronium hexafluorophosphate (COMU); and the like.

A single macrocyclic crosslink is typically formed by substituting the non-natural amino acid S-pentylalanine at i, i+4 positions, resulting in a three amino acid gap flanking the macrocyclic crosslink. Two macrocyclic crosslinks within a single polypeptide are typically formed by substituting R-octenylalanine/S-pentenylalanine or S-octenylalanine/R-pentenylalanine at i, i−7 positions.

Ring closing metathesis is typically catalyzed by a ruthenium and/or molybdenum catalyst. Such a catalyst could be a first generation Grubb's catalyst (which consists of a ruthenium molecule substituted with two phosphine groups, two chlorine atoms, and a carbene compound), a second generation Grubb's catalyst (which is like the first generation Grubb's catalyst but consists of an N-Heterocyclic carbene in place of the phosphine groups), or a Schrock catalyst (which is a molybdenum-based catalyst).

Such polypeptides may exhibit increased stabilization of an α-helical conformation and protease resistance. Other means for stabilizing an α-helix include other ring-forming reactions such as: copper catalyzed azide alkyne cycloaddition (CuAAC), lactamization, cysteine-xylene stapling, cysteine-perfluorobenzene stapling, thiol-yne/-ene Click chemistry, selenocysteine stapling, tryptophan condensation, and C—H activation. Additionally, an α-helix might be stabilized by adding helical caps between terminal side-chains and the peptide backbone or via electrostatic or hydrogen bonding interactions at select positions.

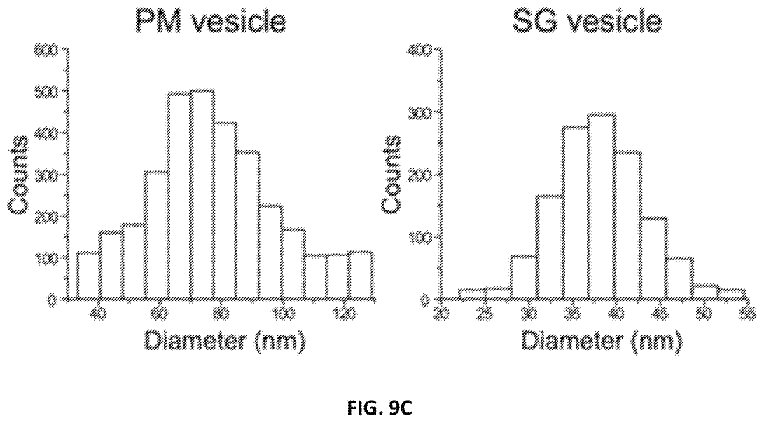

In some instances, the polypeptide constructs described herein do not substantially aggregate, as determined by size exclusion chromatography as shown, for example, in FIG. 6 and described in Example 3. In some embodiments, less than 10% of the polypeptide aggregates (e.g., less than 10%, less than 9%, less than 8%, less than 7%, less than 6%, less than 5%, less than 4%, less than 3%, less than 2%, less than 1%). In some instances, the polypeptide constructs described herein bind to the C2B domain of Syt1 or Syt2 with a K_{D} of around 24 μM to around 35 μM, as shown, for example, in EXTENDED DATA FIG. 2 and described in Example 3. In some embodiments, the polypeptide constructs described herein binds to a wild-type Syt1 or Syt2 C2B domain but not a mutant Syt1 C2B domain comprising mutations R281A, E295A, Y338W, R398A, and R399A (e.g., Syt1 QM), as described in Example 3 (also see EXTENDED DATA FIG. 2 of U.S. Provisional Patent Application Ser. No. 63/311,001). In some embodiments, the polypeptide constructs described herein block Ca²⁺-dependent binding of a Syt2 homolog to a SNARE peptide.

### III. Syt2 Expression Inhibitors

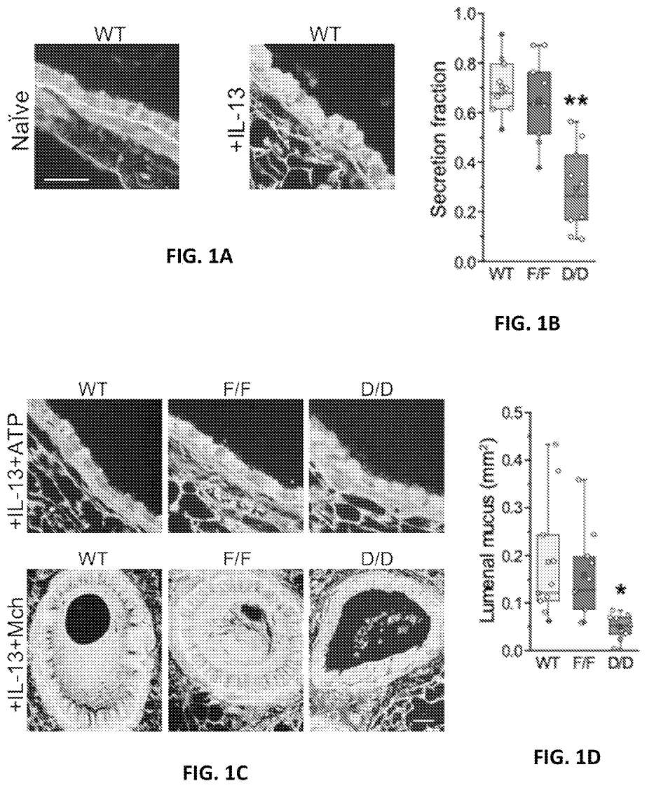

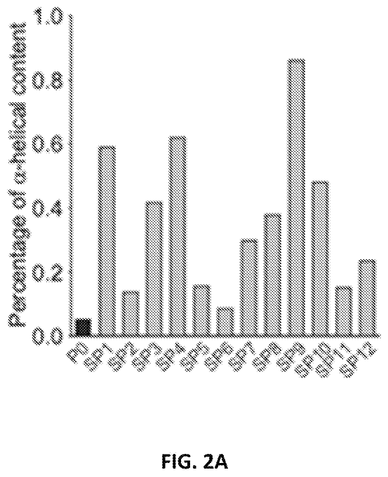

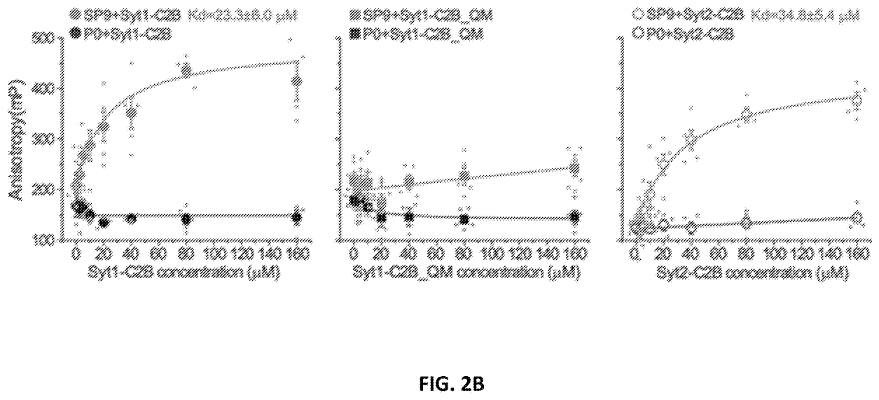

As described in FIG. 1, deletion of SYT2 blocks secretion of intracellular mucin in mouse bronchial airways after IL-3 and ATP stimulation. Therefore, inhibition of SYT2 expression in airway epithelial cells, such as airway secretory cells, is another approach for treating obstruction of bronchial airways in a subject as described herein. Inhibition of SYT2 expression may be accomplished by administering a polynucleotide (e.g., oligonucleotide) to the subject to decrease or inhibit the expression of the SYT2 gene. In some embodiments, the polynucleotide may be, for example, a DNA oligonucleotide or an RNA oligonucleotide. In some embodiments, the oligonucleotide may be used in a gene editing system. An oligonucleotide that inhibits or decreases the expression of the SYT2 gene may knock out or knock down the SYT2 gene (e.g., in an airway epithelial cell such as, for example, an airway secretory cell) in the subject. Polynucleotide compositions as provided herein can be designed based on the genomic or transcript sequences of the SYT2 gene. The human SYT2 gene is located at chromosome 1q32.1. Information about the SYT2 gene and protein are set forth under NCBI Gene ID 127833 and under UniProt Accession No. Q8N9I0.

In some embodiments, the mRNA transcript of the SYT2 gene may be targeted for cleavage and degradation. Different portions of the mRNA transcript may be targeted to decrease or inhibit the expression of the SYT2 gene. In some embodiments, a DNA oligonucleotide may be used to target the mRNA transcript and form a DNA:RNA duplex with the mRNA transcript. The duplex may then be recognized and the mRNA cleaved by specific proteins in the cell. In other embodiments, an RNA oligonucleotide may be used to target the mRNA transcript of the SYT2 gene. In some embodiments, the oligonucleotide may be a shRNA, an ASOs, or an miRNA. In some embodiments, the oligonucleotide may mediate an RNase H-dependent cleavage of the mRNA transcript of the SYT2 gene.

A short hairpin RNA or small hairpin RNA (shRNA) is an artificial RNA molecule with a hairpin turn that can be used to silence target gene expression via the small interfering RNA (siRNA) it produced in cells. See, e.g., Fire et. al., Nature 391:806-811, 1998; Elbashir et. Al., Nature 411:494-498, 2001; Chakraborty et al., Mol Ther Nucleic Acids 8:132-143, 2017; Bouard et al., Br. J. Pharmacol. 157:153-165, 2009. Expression of shRNA in cells is typically accomplished by delivery of plasmids or through viral or bacterial vectors. Suitable bacterial vectors include but not limited to adeno-associated viruses (AAVs), adenoviruses, and lentiviruses (as discussed further in Section IV). Once the vector has integrated into the host genome, the shRNA is then transcribed in the nucleus by polymerase II or polymerase III depending on the promoter choice. The resulting pre-shRNA is exported from the nucleus and then processed by Dicer and loaded into the RNA-induced silencing complex (RISC). The sense strand is degraded by RISC and the antisense strand directs RISC to an mRNA that has a complementary sequence. A protein called Ago2 in the RISC then cleaves the mRNA, or in some cases, represses translation of the mRNA, thus, leading to its destruction and an eventual reduction in the protein encoded by the mRNA. Thus, the shRNA leads to targeted gene silencing. shRNA is an advantageous mediator of siRNA in that it has relatively low rate of degradation and turnover.

In some embodiments, the methods described herein for treating airway obstruction in a subject comprise administering an shRNA to the subject. The methods may comprise administering to the subject a therapeutically effective amount of a vector, wherein the vector comprises a polynucleotide encoding an shRNA capable of hybridizing to a portion of an mRNA transcript of the SYT2 gene. In some embodiments, the vector may also include appropriate expression control elements known in the art, including, e.g., promoters (e.g., tissue specific promoters), enhancers, and transcription terminators. In particular, expression of the shRNA can be driven by a promoter solely or particularly active in airway epithelial cells such as, for example airway secretory cells. Such promoters include, for example, ScblA1 (also known as CCSP), cytokeratin 5 (CK5), Sox2, and Sonic Hedgehog (Shh). See, e.g., Li, H. et al. Cre-mediated recombination in mouse Clara cells. Genesis 46, 300-307, (2008).

Once the vector is delivered to the airway epithelial cell, the shRNA may be integrated into the cell's genome and undergo downstream processing by Dicer and RISC (described in detail further herein) to eventually hybridize to the mRNA transcript of the SYT2 gene, leading to mRNA cleavage and degradation.

The present disclosure also provides siRNA-based therapeutics for inhibiting expression of SYT2 in a subject. The double stranded RNAi therapeutic includes a sense strand complementary to an antisense strand. The sense or antisense strands of the siRNA may be about 14, 15, 16, 17, 18, 19, 20, 21, 22, 23, 24, 25, 26, 27, 28, 29, or 30 nucleotides in length. The antisense strand of the siRNA-based therapeutic includes a region complementary to a part of an mRNA encoding SYT2. Additional methods to make therapeutic siRNA can be found in U.S. Pat. No. 9,399,775, which is incorporated by reference in its entirety for all purposes.

RNase H-dependent antisense oligonucleotides (ASOs) are single-stranded, chemically modified oligonucleotides that bind to complementary sequences in target mRNAs and reduce gene expression both by RNase H-mediated cleavage of the target RNA and by inhibition of translation by steric blockade of ribosomes. In some embodiments, to target the mRNA transcript of the SYT2 gene for degradation, a nucleic acid (e.g., DNA oligonucleotide) capable of hybridizing to a portion of the mRNA may be administered to the subject. Once inside the cell (e.g., an airway epithelial cell), the DNA oligonucleotide base pairs with its targeted mRNA transcript. RNase H may bind to the resulting duplex and cleave the mRNA transcript at one or more places. The DNA oligonucleotide may further bind to other mRNA transcripts to target them for RNase H degradation reducing the expression of the SYT2 gene.

A microRNA (miRNA) is a small non-coding RNA molecule that functions in RNA silencing and post-transcriptional regulation of gene expression. miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease translation efficiency of the mRNA transcript into proteins by ribosomes. In some embodiments, miRNAs derive from regions of RNA transcripts that fold back on themselves to form short hairpins, which are also called pri-miRNA. Once transcribed as pri-miRNA, the hairpins are cleaved out of the primary transcript in the nucleus by an enzyme called Drosha. The hairpins, or pre-miRNA, are then exported from the nucleus into the cytosol. In the cytosol, the loop of the hairpin is cleaved off by an enzyme called Dicer. The resulting product is now a double strand RNA with overhangs at the 3′ end, which is then incorporated into RISC. Once in the RISC, the second strand is discarded and the miRNA that is now in the RISC is a mature miRNA, which binds to mRNAs that have complementary sequences. In some embodiments, an miRNA targeting the SYT2 gene may be used in methods described herein.

In some embodiments, the knocking out or knocking down of SYT2 gene expression is performed using a gene editing system such as, but not limited to, meganucleases designed against the Syt2 genomic sequence, the CRISPR/Cas system, TALENs, and other technologies for precise editing of genomes; Cre-lox site-specific recombination; FLP-FRT recombination; Bxbl-mediated integration; zinc-finger mediated integration; homologous recombination; prime editing and transposases; translocation; and inversion.

In some embodiments, the Syt2 expression inhibitor comprises one or more gene editing components such as a targeted nuclease and a guide RNA (gRNA). Four classes of CRISPR-based genome editing agents are nucleases, base editors, transposases/recombinases and prime editors. Components of any of these gene editing systems can be used to reduce or eliminate Syt2 expression. As used throughout, the term “targeted nuclease” refers to nuclease that is targeted to a specific DNA sequence in the genome of a cell to produce a strand break at that specific DNA sequence. The strand break can be single-stranded or double-stranded. Targeted nucleases include, but are not limited to, a Cas nuclease, a TAL-effector nuclease and a zinc finger nuclease. As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. Any CRISPR/Cas system that is capable of altering a target polynucleotide sequence in a cell can be used in methods described here. See, for example, Sanders and Joung, Nature Biotechnol 32:347-355, 2014, Huang et al., J Cell Physiol 10:1-17, 2017 and Mitsunobu et al., Trends Biotechnol 17:30132-30134, 2017.

In some embodiments, inhibiting comprises contacting the polynucleotide encoding Syt2 with at least one gRNA and optionally a targeted nuclease, wherein the at least one gRNA comprises a sequence that specifically hybridizes to a genomic sequence of the SYT2 gene In some embodiments, inhibiting comprises mutating the polynucleotide encoding Syt2. In some embodiments, inhibiting comprises contacting the polynucleotide encoding Syt2 with a targeted nuclease. In some embodiments, inhibiting comprises performing clustered regularly interspaced short palindromic repeats (CRISPR)/Cas genome editing.

### IV. Pharmaceutical Compositions

Also provided in the present disclosure are pharmaceutical compositions containing one or more of the polypeptide constructs as described herein and one or more pharmaceutically acceptable excipients. Also provided are pharmaceutical compositions comprising an inhibitor of Syt2 expression as described above in Section III, optionally in combination with one or more pharmaceutically acceptable excipients. The term “active ingredient” is used herein to refer individually and collectively to any of the polypeptide constructs or SYT2 expression inhibitors provided herein.

The pharmaceutical compositions can be prepared by any of the methods well known in the art of pharmacy and drug delivery. In general, methods of preparing the compositions include the step of bringing an active ingredient as described herein and any other additional active ingredients into association with a carrier containing one or more accessory ingredients. The pharmaceutical compositions are typically prepared by uniformly and intimately bringing the active ingredient(s) into association with a liquid carrier or a finely divided solid carrier or both, and then, if necessary, shaping the product into the desired formulation. In addition to an active ingredients described herein in Sections II and III, pharmaceutical compositions provided herein may also contain additional active ingredients such as corticosteroids, anti-inflammatory drugs, agents to improve mucus clearance, and inhibitors of other SNARE complex proteins (e.g., Munc18, VAMP8, Munc13, or Stx3) as described below.

Pharmaceutical compositions containing an active ingredient as described herein can be in the form of aqueous or oleaginous solutions and suspensions (e.g., sterile solutions or suspensions for administration by oral inhalation or as a nasal spray or nasal drops). Such preparations can be formulated using non-toxic parenterally-acceptable vehicles including water, Ringer's solution, and isotonic sodium chloride solution, and acceptable solvents such as 1,3-butane diol. In addition, sterile, fixed oils can be used as a solvent or suspending medium. For this purpose any bland fixed oil can be employed including synthetic monoglycerides, diglycerides, or triglycerides.

Aqueous suspensions can contain one or more of active ingredient as described herein in admixture with excipients including, but not limited to: suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxybenzoate. Dispersible powders and granules (suitable for preparation of an aqueous suspension by the addition of water) can contain an active ingredient as described herein in admixture with a dispersing agent, wetting agent, suspending agent, or combinations thereof. Oily suspensions can be formulated by suspending an an active ingredient as described herein in a vegetable oil (e.g., arachis oil, olive oil, sesame oil or coconut oil), or in a mineral oil (e.g., liquid paraffin). Oily suspensions can contain one or more thickening agents, for example beeswax, hard paraffin, or cetyl alcohol. These compositions can be preserved by the addition of an anti-oxidant such as ascorbic acid.

In certain embodiments, a pharmaceutical composition can be formulated for inhalation. The compositions described herein, either alone or in combination with other suitable components, can be made into aerosol formulations to be administered via inhalation (e.g., intranasally or intratracheally). Aerosol formulations can be placed into pressurized acceptable propellants, such as dichiorodifluoromethane, propane, nitrogen, and the like. In certain embodiments, the pharmaceutical composition can be nebulized. Methods for delivering compositions directly to the lungs via nasal aerosol sprays have been described, e.g., in U.S. Pat. No. 6,565,841. Likewise, the delivery of drugs using intranasal microparticle resins and lysophosphatidyl-glycerol compounds (U.S. Pat. No. 5,725,871) are also well-known in the pharmaceutical arts. Similarly, transmucosal drug delivery in the form of a polytetrafluoroetheylene support matrix is described in U.S. Pat. No. 5,780,045.

In some instances, the pharmaceutical composition can be formulated as a solid particulate. In some instances, the pharmaceutical composition can be can be formulated as a liquid. In some instances, the pharmaceutical composition can be formulated as a polymeric nanopartical or a lipid nanoparticle. A polymeric nanoparticle formed from natural or synthetic materials can comprise, for example, a polymersome, a dendrimer, a polymer micelle, or a nanosphere. A lipid-based nanoparticle can comprise, for example, a liposome or a lipid nanoparticle. Liposomes comprise phospholipids that form vesicular structures, whereas lipid nanoparticles comprise miceller structures within the particle core.

The active ingredients, and pharmaceutical compositions thereof, may be administered to a subject by any technique known in the art, including local or systemic delivery. Routes of administration include, but are not limited to, intrapulmonary inhalation, oral inhalation administration, and intranasal administration. Inhalation delivery can enhance targeting a pharmaceutical composition to airway epithelial cells. See, e.g., Manunta et al., Airway Deposition of Nebulized Gene Delivery Nanocomplexes Monitored by Radioimaging Agents, Am J Respir Cell Mol Biol 49(3): 471-480 (2013).

Depending on whether transient or stable expression is desired one can select an appropriate delivery vector for delivery of the polynucleotides provided herein for inhibition of Syt2 expression. Examples of delivery vectors that may be used with the present disclosure are viral vectors, plasmids, exosomes, liposomes, bacterial vectors, or nanoparticles. The present disclosure also provides for delivery by any means known in the art, some of which are described in Fumoto et al. (2021), which is hereby incorporated by reference in its entirety for all purposes.

For targeted delivery to epithelial cells, one skilled in the art would appreciate that some delivery vectors may be genetically modified to target a specific cell type or to tissue type. In addition, depending on the required therapeutic duration a viral delivery vector can be genetically modified to be continuously replicating, replication-defective, or conditionally replicating as described in, Sliva, K. and Schnierle, B. S., Selective gene silencing by viral delivery of short hairpin RNA Virology Journal (2010).

In some embodiments, the SYT2 shRNA or siRNA can be delivered by vector comprising an adenovirus vector, an adeno-associated viral vector, a retrovirus vector, a lentivirus vector, or a nanoparticle. Examples of nanoparticles that can be use with the present disclosure, include but are not limited to, exosomes, liposomes, organic nanoparticles, or inorganic nanoparticles. Other non-limiting examples of nanoparticles include, but are not limited to, e.g., those provided in Hong and Nam, Functional Nanostructures for Effective Delivery of Small Interfering RNA Therapeutics, Theranostics 4.12: 1211-1232 (2014), which is hereby incorporated by reference in its entirety for all purposes. In some embodiments, the siRNA active ingredient is administered in a solution. The siRNA may be administered in an unbuffered solution. In one embodiment, the siRNA is administered in water. In other embodiments, the siRNA is administered with a buffer solution, such as an acetate buffer, a citrate buffer, a prolamine buffer, a carbonate buffer, or a phosphate buffer or any combination thereof. In some embodiments, the buffer solution is phosphate buffered saline.

In some embodiments, where the Syt2 expression inhibitor comprises one or more gene editing components including a targeted nuclease, the targeted nuclease can be introduced into a cell in polypeptide form. In certain embodiments, the targeted nuclease may be conjugated to a cell-penetrating polypeptide. Non-limiting examples of cell-penetrating peptides include, but are not limited to, e.g., those provided in Milletti et al., Drug Discov. Today 17: 850-860, 2012, the relevant disclosure of which is hereby incorporated by reference in its entirety. In some embodiments, the target motif in the SYT2 gene, to which the targeted nuclease is directed by a polynucleotide (e.g., a guide RNAs). The sgRNAs can be selected depending on the particular CRISPR/Cas system employed, and the sequence of the target polynucleotide, as will be appreciated by those skilled in the art. In some instances, the targeted nuclease and the sgRNA can be provide as separate the pharmaceutical compositions or can be provided together in a single pharmaceutical composition (e.g., a polynucleotide complex or ribonucleoprotein (RNP)). As used herein, the phrase “introducing” in the context of introducing a polynucleotide or a polynucleotide complex comprising a nucleic acid and a polypeptide refers to the translocation of the nucleic acid sequence or the polynucleotide complex from outside a cell to inside the cell. In some cases, introducing refers to translocation of the nucleic acid or the complex from outside the cell to inside the nucleus of the cell.

Pharmaceutical compositions of the present disclosure may be packaged as a single use “unit dose” container or as a multi-dose container. In some instances, a unit dose of the compositions described in this disclosure is provided. Examples of single use containers are blister packs or capsules. Examples of multi-dose containers are drop dispensers, or vials. Kits according to the present disclosure may include one or more unit doses of a composition and a device for administering the composition. Kits may include a single use “unit dose” container or a multi-dose container. Examples of single use containers are blister packs or capsules. Examples of multi-dose containers are drop dispensers, or vials. In some instances, the device for administering the composition may be an aerosolization device. For example, in some instances, the device may be an aerosolizer, an inhaler, or a nebulizer. Inhalers include metered dose inhalers (MDIs), dry powder inhalers (DPIs), and soft mist inhalers (SMIs). Other devices for aerosolization of liquid compositions are well-known in the art. In some instances, the kits may include a device for administrating the composition via injection. For example, the kits may include one or more syringes. In another example, the kits may include one or more needles. In another example, the kits may include one or more syringes and one or more needles. The kits may also include a pump or a pen device for administering the composition via injection. In some instances, the kit may include instructions describing use of the device to administer the composition.

### V. Methods for Inhibiting Mucin Secretion

Also provided herein are methods of inhibiting mucin secretion in an airway epithelial cell. In some embodiments, the airway epithelial cell is a airway secretory cell. Further provided herein are methods of inhibiting Syt2-mediated stimulated mucin secretion, triggered by Ca²⁺ release after ATP or methacholine bind to hepta-helical PM receptors coupled to Gq. In some instances, the methods include contacting the cell with an effective amount of at least one polypeptide construct as described above in Section II. In some instances, the methods include contacting the cell with an effective amount of an inhibitor of SYT2 expression as described above in Section III thereby reducing Syt2 expression in the cell. In some instances, the methods comprise contacting the cell with both at least one polypeptide construct and an inhibitor of SYT2 expression. “Contacting” cells may include addition of a polypeptide construct to a cell culture in vitro, or administering a polypeptide construct to a subject (e.g., in conjunction with a pharmaceutical composition as described above).

Also provided herein is a method of treating a subject having mucus hypersecretion-based airway obstruction and/or mucus occlusions by administering an effective amount of at least one polypeptide construct or pharmaceutical composition thereof as described above. Also provided herein are methods of treating a subject having mucus hypersecretion-based airway obstruction and/or mucus occlusions by administering an effective amount of an inhibitor of Syt2 expression or pharmaceutical composition thereof as described above. In some instances, the methods comprising administereing an effective amount of at least one polypeptide construct or pharmaceutical composition thereof and an effective amount of an inhibitor of Syt2 expression.

In some embodiments, the subject has one or more of a respiratory viral infection, asthma, chronic obstructive pulmonary disease (COPD), or cystic fibrosis. In some instances, treatment may further comprise administering a therapeutically effective amount of an inhibitor of at least one of Munc18, VAMP8, Munc13, or Stx3.

Mucus dysfunction is a very common cause of symptoms and disease progression in common diseases of the airways. Rapid mucin secretion is a major contributor to airway mucus dysfunction, yet there are no currently available therapies directed at this root cause. Patients with asthma (8% of Americans), COPD (6% of American adults), cystic fibrosis (1/3,000 white newborns), and bronchiectasis (139/100,000 adult Americans) could potentially benefit from this invention. In general, it could be anticipated that the greatest need would be among patients with persistent mucus plaques and plugs, and those with acute airflow obstruction due to mucus obstruction of the airways.

In some embodiments, the polypeptide constructs described herein reduce fractional secretion of intracellular mucin stimulated by methacholine by up to about 5%, 10%, 15%, 20%, 25%, 30%, 35%, 40%, 50%, 55%, 60%, 65%, 70%, 75%, 80%, 85%, or 90% when administered to an airway epithelial cell.

In some embodiments, administration of the polypeptide constructs described herein reduces airway luminal mucus accumulation in the lung by at least about 10% (e.g., at least 10%, 15%, 20%, 25%, 30%, 35%, or 40%) when administered to an airway epithelial cell as described, for example, in Example 8.

Polypeptide constructs provided herein can be used acutely and chronically. In the acute setting, the rapid release of hyperproduced mucins is well-known to be the major cause of airflow obstruction and death in asthma, and plays a similar role in other airway diseases such as acute exacerbations of COPD, cystic fibrosis, and bronchiectasis. Administration of a polypeptide construct as provided herein could prevent further acute mucus occlusion, while other interventions such as corticosteroids can be administered to prevent further mucin production. Thus, in some embodiments, the polypeptide constructs provided herein are administered to the subject pro re nata (i.e., on an as needed basis). In the chronic setting, where persistent mucus plaques cause chronic airway obstruction and inflammation in asthma, COPD, cystic fibrosis, and bronchiectasis, the polypeptide construct as provided herein could be administered alongside anti-inflammatory drugs (to reduce mucin production) and agents such as hypertonic saline solution (to improve mucus clearance) to prevent further plaque formation. Thus, in some embodiments, the polypeptide constructs provided herein are administered to the subject on a regular dosing regimen (e.g., each at least once per day). Together, such a strategy might improve the therapy of these undertreated airway diseases. Examples of methods of administration of the formulation are generally described under “Pharmaceutical Compositions” above.

In some embodiments, the subject may also be administered a therapeutically effective amount of an inhibitor of at least one of Munc18, VAMP8, Munc13, or Stx3. Each of these proteins are implicated in basal and/or stimulated mucin secretion and are either a component of the SNARE complex that includes SNAP-23 or affects its function. For example, Stx3 and VAMP8 are components of the SNARE complex containing SNAP-23. Furthermore, Munc18-2 mutant mice are selectively impaired in stimulated mucin secretion. Stx2 binds and colocalizes with Munc18-2, and Munc18-2 overexpression reduces the amount of Stx3 associated with SNAP-23. Also, the importance of these proteins in mucin secretion can be inferred by the role of homologous SNARE protein complex components, synaptotagmins, and Munc proteins involved in neurotransmitter secretion. Example of such inhibitors are 2-amiinobenzothiazoles tht inhibit Munc13-4 (Bruinsma et al., 2018).

In certain embodiments, an active ingredient or a pharmaceutical composition as provided herein are administered to the lungs of the subject (i.e. pulmonary administration). The term “pulmonary administration” represents any method of administration in which an active agent can be administered through the pulmonary route by inhaling or otherwise administering into the lungs an aerosolized liquid or powder form (nasally or orally). Such aerosolized liquid or powder forms are traditionally intended to substantially release and or deliver the active agent to the epithelium of the lungs. In certain embodiments, the active agent is in liquid form. Pulmonary administration is further described in International Application Publication No. WO/1994/020069, which describes pulmonary delivery of chemically modified proteins.

The active ingredients, and pharmaceutical compositions thereof, may be administered in a single dose or in multiple doses (e.g., two, three, or more single doses per treatment) over a time period (e.g., hours or days). The active ingredients described herein, and pharmaceutical compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of active ingredient administered will depend upon a variety of factors, including, for example, the particular indication being treated, the mode of administration, whether the desired benefit is prophylactic or therapeutic, the severity of the indication being treated and the age and weight of the patient, the bioavailability of the particular active ingredient, the conversation rate and efficiency into active drug compound under the selected route of administration, etc.

Determination of an effective dosage of an active ingredient for a particular use and mode of administration is well within the capabilities of those skilled in the art. Effective dosages may be estimated initially from in vitro activity and metabolism assays. For example, an initial dosage of a polypeptide construct for use in animals may be formulated to achieve a circulating blood or serum concentration that is at or above an IC₅₀ of the particular polypeptide construct as measured in an in vitro assay. The dosage can be calculated to achieve such circulating blood or serum concentrations taking into account the bioavailability of the particular polypeptide construct via the desired route of administration. Initial dosages of compound can also be estimated from in vivo data, such as animal models. For example, an average mouse weighs 0.025 kg. Administering 0.025, 0.05, 0.1 and 0.2 mg of a polypeptide constructs per day may therefore correspond to a dose range of 1, 2, 4, and 8 mg/kg/day. If an average human adult is assumed to have a weight of 70 kg, the corresponding human dosage would be 70, 140, 280, and 560 mg of the polypeptide construct per day. Dosages for other active agents may be determined in similar fashion. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the polypeptide construct or other active compound, the bioavailability of the polypeptide construct or other active compound, its metabolism kinetics and other pharmacokinetic properties, the mode of administration and various other factors, discussed above. The dose of the polypeptide construct can be, for example, about 0.01-750 mg/kg, or about 0.01-500 mg/kg, or about 0.01-250 mg/kg, or about 0.01-100 mg/kg, or about 0.1-50 mg/kg, or about 1-25 mg/kg, or about 1-10 mg/kg, or about 5-10 mg/kg, or about 1-5 mg/kg. The dose of the polypeptide construct can be about 0.01, 0.05, 0.1, 0.5, 1, 5, 10, 15, 20, 25, 30, 35, 40, 45, 50, 55, 60, 65, 70, 75, 80, 85, 90, 95, or 100 mg/kg.

Dosage amount and interval may be adjusted individually to provide levels of the an active ingredient that is sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of teh an active ingredient may not be related to plasma concentration. The dosage may be adjusted by the individual physician in the event of any complication.

The compounds may be present in a therapeutically effective concentration. In certain embodiments, the concentration of said compound is about 0.1 nmol/L to about 1000 nmol/L at the time of administration; e.g., about 0.1 nmol/L to about 500 nmol/L, or about 0.1 nmol/L to about 250 nmol/L, or about 0.1 nmol/L to about 100 nmol/L, or about 0.1 nmol/L to about 50 nmol/L, or about 0.1 nmol/L to about 10 nmol/L, or about 0.1 nmol/L to about 1 nmol/L, or about 1 nmol/L to about 500 nmol/L, or about 1 nmol/L to about 250 nmol/L, or about 1 nmol/L to about 100 nmol/L, or about 1 nmol/L to about 50 nmol/L, or about 1 nmol/L to about 10 nmol/L, or about 10 nmol/L to about 500 nmol/L, or about 10 nmol/L to about 250 nmol/L, or about 10 nmol/L to about 100 nmol/L, or about 10 nmol/L to about 50 nmol/L, or about 100 nmol/L to about 500 nmol/L, or about 100 nmol/L to about 250 nmol/L. One of skill in the art will recognize that suitable volume of the dose may be selected based on the desired route of administration.

### Exemplary Embodiments

As used below, any reference to a series of embodiments is to be understood as a reference to each of those embodiments disjunctively (e.g., “Embodiments 1-4” is to be understood as “Embodiments 1, 2, 3, or 4”).

Embodiment 1 is a polypeptide comprising a SNAP-25A peptide, or homolog or variant thereof, attached to a cell penetrating peptide, the peptide comprising a first pair of non-natural amino acids comprising a macrocyclic crosslink.

Embodiment 2 is a polypeptide comprising a SNAP-25A peptide, or homolog or variant thereof, attached to a non-biological material, the peptide comprising a first pair of non-natural amino acids comprising a macrocyclic crosslink.

Embodiment 3 is a polypeptide comprising a first peptide having at least 64% identity to SEQ ID NO: 1 or SEQ ID NO: 2 attached to a cell penetrating peptide, the peptide comprising a first pair of non-natural amino acids comprising a macrocyclic crosslink.

Embodiment 4 is a polypeptide comprising a first peptide having at least 64% identity to SEQ ID NO: 1 or SEQ ID NO: 2 attached to a a non-biological material, the peptide comprising a first pair of non-natural amino acids comprising a macrocyclic crosslink.

Embodiment 5 is a polypeptide of any one of embodiments 1-4, wherein the first peptide comprises a second pair of non-natural amino acids comprising a macrocyclic crosslink.

Embodiment 6 is a polypeptide of any one of embodiments 1-4, wherein the first pair of non-natural amino acids flanks six contiguous amino acid residues in the peptide.

Embodiment 7 is a polypeptide of embodiment 5, wherein the first pair of non-natural amino acids and second pair of non-natural amino acids each flank three contiguous amino acid residues in the peptide, and wherein the three contiguous amino acid residues flanked by the first pair of non-natural amino acids are different than the three contiguous amino acid residues flanked by the second pair of non-natural amino acids.

Embodiment 8 is a polypeptide of any one of embodiments 1-4 or 6, wherein the first pair of non-natural amino acids correspond to amino acids 6 and 13, 7 and 14, 10 and 17, or 11 and 18 in SEQ ID NO: 1 or SEQ ID NO: 2.

Embodiment 9 is a polypeptide of any one of embodiments 1-4 or 7, wherein the first pair of non-natural amino acids correspond to amino acids 3 and 7, 6 and 10, or 7 and 11 in SEQ ID NO: 1 or SEQ ID NO: 2.

Embodiment 10 is a polypeptide according to any one of embodiments 1-4, 7, or 9, wherein the second pair of non-natural amino acids correspond to amino acids 10 and 14, 13 and 17, 14 and 18, or 17 and 21 in SEQ ID NO: 1 or SEQ ID NO: 2.

Embodiment 11 is a polypeptide according to any one of embodiments 1 to 10, wherein the macrocyclic crosslink is positioned on a non-binding side of the first peptide, wherein the non-binding side of the first peptide does not contain amino acid residues that interact with amino acid residues in the C2B domain of a synaptotagmin protein.

Embodiment 12 is a polypeptide according to any one of embodiments 1-4, 6, 8, or 11, wherein the first peptide comprises any one of SEQ ID NO: 3 or 4.

Embodiment 13 is a polypeptide according to any one of of embodiments 1-5, 7, 9, 10, or 11, wherein the first peptide comprises any one of SEQ ID NO: 5, 6, 7, 8, or 9.

Embodiment 14 is a polypeptide of embodiment 13, comprising SEQ ID NO: 8.

Embodiment 15 is a polypeptide according to any one of embodiments 1 to 14, wherein the cell penetrating peptide comprises at least one of the cell penetrating peptides listed in Table 1.

Embodiment 16 is a polypeptide according to any one of embodiments 1 to 15, wherein the cell penetrating peptide comprises penetratin.

Embodiment 17 is a polypeptide according to any one of embodiments 1 to 16, wherein the cell penetrating peptide is not an HIV-1 TAT peptide.

Embodiment 18 is a polypeptide according to any one of embodiments 1 to 17, wherein the C-terminus of the cell penetrating peptide is linked to the N-terminus of the peptide.

Embodiment 19 is a polypeptide construct as described herein comprising at least one chemically modified amino acid or at least one conservative amino acid substitution.

Embodiment 20 is a polypeptide construct as described herein that does not substantially aggregate, as determined by size exclusion chromatography.

Embodiment 21 is a polypeptide construct as described herein that binds to the C2B domain of Syt1 or Syt2 with a K_{D} of around 24 μM to around 35 μM.

Embodiment 22 is a polypeptide construct as described herein that binds to the of a wild-type Syt1 or Syt2 C2B domain but not a mutant Syt1 C2B domain comprising mutations R281A, E295A, Y338W, R398A, and R399A.

Embodiment 23 is a polypeptide construct as described herein that blocks Ca²⁺-dependent binding of a Syt2 homolog to a SNARE peptide.

Embodiment 24 is a pharmaceutical composition comprising a polypeptide according to any one of the preceding embodiments and a pharmaceutically acceptable excipient.

Embodiment 25 is a method of treating a subject having mucus hypersecretion-based airway obstruction, the method comprising administering to the subject a therapeutically effective amount of a polypeptide or pharmaceutical composition according to any one of the preceding embodiments.

Embodiment 26 is a method according to embodiment 25, wherein the subject has a respiratory viral infection, asthma, chronic obstructive pulmonary disease (COPD), or cystic fibrosis.

Embodiment 27 is a method according to embodiments 25 or 26, wherein the subject has developed mucus occlusions.

Embodiment 28 is a method according to any one of embodiments 25 to 27, further comprising administering a therapeutically effective amount of an inhibitor of at least one of Munc18, VAMP8, Munc13, or Stx3.

Embodiment 29 is a method of inhibiting mucin secretion in an airway epithelial cell, the method comprising contacting the airway epithelial cell or a cell derived from an airway epithelial cell with a polypeptide or pharmaceutical composition according to any one of the preceding embodiments.

Embodiment 30 is a method of inhibiting Syt2-mediated stimulated mucin secretion in an airway epithelial cell or a cell derived from an airway epithelial cell, triggered by Ca²¹ release after ATP or methacholine bind to hepta-helical PM receptors coupled to Gq, the method comprising contacting the cell with a polypeptide or pharmaceutical composition according to any one of the preceding embodiments.

Embodiment 31 is a method according to any one of embodiments 25 to 30 wherein administration of the polypeptide or pharmaceutical composition reduces fractional secretion of intracellular mucin stimulated by methacholine by up to about 5%, 10%, 15%, 20%, 25%, 30%, 35%, 40%, 50%, 55%, 60%, 65%, 70%, 75%, 80%, 85%, or 90% when administered to an airway epithelial cell.

Embodiment 32 is a method according to any one of embodiments 25 to 31 wherein administration of the polypeptide or pharmaceutical composition as described herein reduces airway luminal mucus accumulation in the lung by at least about 10% (e.g., at least 10%, 15%, 20%, 25%, 30%, 35%, or 40%) when administered to an airway epithelial cell.

Embodiment 33 is a polynucleotide that inhibits expression of SYT2.

Embodiment 34 is a polynucleotide according to embodiment 30, wherein the polynucleotide is a SYT2 targeting siRNA, shRNA, ASO, miRNA, or CRISPR/Cas system guide RNA.

Embodiment 35 is a pharmaceutical composition comprising a polynucleotide according to embodiment 33 or 34 and a pharmaceutically acceptable excipient.

Embodiment 36 is a method of treating a subject having mucus hypersecretion-based airway obstruction, the method comprising administering to the subject a therapeutically effective amount of a Syt2 expression inhibitor according to embodiment 33 or 34 or a pharmaceutical composition thereof according to embodiment 35.

Embodiment 37 is a method of inhibiting mucin secretion in an airway epithelial cell, the method comprising contacting the airway epithelial cell or a cell derived from an airway epithelial cell with a Syt2 expression inhibitor according to embodiment 33 or 34 or a pharmaceutical composition thereof according to embodiment 35.

Embodiment 38 is a method of inhibiting Syt2-mediated stimulated mucin secretion in an airway epithelial cell or a cell derived from an airway epithelial cell, triggered by Ca²⁺ release after ATP or methacholine bind to hepta-helical PM receptors coupled to Gq, the method comprising contacting the cell with a Syt2 expression inhibitor according to embodiment 33 or 34 or a pharmaceutical composition thereof according to embodiment 35.

Embodiment 39 is a method according to any one of embodiments 36 to 38, wherein the Syt2 expression inhibitor is a polynucleotide or polynucleotide complex.

Embodiment 40 is a method according to any one of embodiments 36 to 39, wherein the Syt2 expression inhibitor is a shRNA, an siRNA, or an miRNA.

Embodiment 41 is a method according to any one of embodiments 36 to 39, wherein the Syt2 expression inhibitor is one or more gene editing system components.

Embodiment 42 is a method according to embodiment 41, wherein the one or more gene editing system components comprise at least one of a targeted nuclease or a guide RNA.

References referred to in this disclosure include:
1. Abdullah, L. H., Wolber, C., Kesimer, M., Sheehan, J. K. & Davis, C. W. Studying Mucin Secretion from Human Bronchial Epithelial Cell Primary Cultures Luhna. 842, (Humana Press, 2012).
2. Alfaro V Y, et al. Safety, tolerability, and biomarkers of the treatment of mice with aerosolized Toll-like receptor ligands. Front Pharmacol. 5, 8 (2014).
3. Basu, J. et al. A minimal domain responsible for Munc13 activity. Nat. Struct. Mol. Biol. 12, 1017-8 (2005).
4. Bird, G. H. et al. Hydrocarbon double-stapling remedies the proteolytic instability of a lengthy peptide therapeutic. Proc. Natl. Acad. Sci. U.S.A 107, 14093-14098 (2010).
5. Bossé, Y., Riesenfeld, E. P., Pare, P. D. & Irvin, C. G. It's Not All Smooth Muscle: Non-Smooth-Muscle Elements in Control of Resistance to Airflow. Annu. Rev. Physiol. 72, 437-462 (2010).
6. Boucher, R. C. Muco-Obstructive Lung Diseases. N. Engl. J. Med. 380, 1941-1953 (2019).
7. Brooks, B. R. et al. CHARMM: The biomolecular simulation program. J. Comput. Chem. 30, 1545-1614 (2009).
8. Bruinsma, James, D., Serrano, M. Q., Esquibel, J., Woo, S. S., Kieler-Grevstad, E., Qurashi, R. Kowalchyk, J. A., and Martin, T. F. J., Small molecules that inhibit the late stage of Munc13-4-dependent secretory granule exocytosis in mast cells J. Biol. Chem. 293, 8217-29 (2018).
9. Brunger, A. T., Choi, U. B., Lai, Y., Leitz, J. & Zhou, Q. Molecular Mechanisms of Fast Neurotransmitter Release. Annu. Rev. Biophys. 47, 469-497 (2018).
10. Choi, U. B. et al. NSF-mediated disassembly of on- and off-pathway SNARE complexes and inhibition by complexin. Elife 7, (2018).
11. Cleaver J O, et al. Lung epithelial cells are essential effectors of inducible resistance to pneumonia. Mucosal Immunol. 7:78-88 (2014).
12. Davis, C. W. & Dickey, B. F. Regulated Airway Goblet Cell Mucin Secretion. Annu. Rev. Physiol. 70, 487-512 (2008).
13. Diao, J. et al. A single-vesicle content mixing assay for SNARE-mediated membrane fusion. Nat. Commun. 1, 54 (2010).
14. Durairaj L. et al., Safety assessment of inhaled xylitol in mice and healthy volunteers. Respir Res, 5:13 (2004).
15. Evans, C. M. et al. The polymeric mucin Muc5ac is required for allergic airway hyperreactivity. Nat. Commun. 6, 6281 (2015).
16. Evans, J. H. & Sanderson, M. J. Intracellular calcium oscillations induced by ATP in airway epithelial cells. Am. J Physiol. Cell. Mol. Physiol. 277, L30-L41 (1999).
17. Evans S E, et al. Inhaled innate immune ligands to prevent pneumonia. Br J Pharmacol, 163:195-206 (2011).
18. Fahrer, J. et al. C2-streptavidin mediates the delivery of biotin-conjugated tumor suppressor protein P53 into tumor cells. Bioconjug. Chem. 24, 595-603 (2013).
19. Fahy, J. V. & Dickey, B. F. Airway Mucus Function and Dysfunction. N. Engl. J. Med. 363, 2233-2247 (2010).
20. Fellermann, M. et al. Super-resolution microscopy unveils transmembrane domain-mediated internalization of cross-reacting material 197 into diphtheria toxin-resistant mouse J774A.1 cells and primary rat fibroblasts in vitro. Arch. Toxicol. (2020). doi:10.1007/s00204-020-02731-4.
21. Gerald, K. Cell and Molecular Biology (4^{th} ed.). John Wiley & Sons. (2002).
22. Goldblatt, D. L. et al. Inducible epithelial resistance against acute Sendai virus infection prevents chronic asthma-like lung disease in mice. Br. J Pharmacol. bph.14977 (2020). doi:10.1111/bph.14977.
23. Guidotti, G., Brambilla, L. & Rossi, D. Cell-Penetrating Peptides: From Basic Research to Clinics. Trends Pharmacol. Sci. 38, 406-424 (2017).
24. Harrison, S. C. Viral membrane fusion. Virology 479-480, 498-507 (2015).
25. Hays, S. R. & Fahy, J. V. The role of mucus in fatal asthma. Am. J. Med. 115, 68-69 (2003).
26. Humphrey, W., Dalke, A. & Schulten, K. VMD: Visual molecular dynamics. J. Mol. Graph. 14, 33-38 (1996).
27. Jaramillo, A. M., Azzegagh, Z., Tuvim, M. J. & Dickey, B. F. Airway mucin secretion. Ann. Am. Thorac. Soc. 15, S164-5170 (2018).
28. Jaramillo, A. M. et al. Different Munc18 proteins mediate baseline and stimulated airway mucin secretion. JCI Insight 4, (2019).
29. Jones, L. C. C. et al. VAMP8 is a vesicle SNARE that regulates mucin secretion in airway goblet cells. J Physiol 5903, 545-561 (2012).
30. Kilby, J. M. et al. Potent suppression of HIV-1 replication in humans by T-20, a peptide inhibitor of gp41-mediated virus entry. Nat. Med. 4, 1302-1307 (1998).
31. Kim, K. et al. Munc18b is an essential gene in mice whose expression is limiting for secretion by airway epithelial and mast cells. Biochem. J. 446, 383-394 (2012).
32. Kim, T. D., Eddlestone, G. T., Mahmoud, S. F., Kuchtey, J. & Fewtrell, C. Correlating Ca²⁺ Responses and Secretion in Individual RBL-2H3 Mucosal Mast Cells. J. Biol. Chem. 272, 31225-31229 (1997).
33. Knust J., et al., Stereological estimates of alveolar number and size and capillary length and surface area in mice lungs. Anat Rec, 292:113, (2009).
34. Kyoung, M. et al. In vitro system capable of differentiating fast Ca²⁺-triggered content mixing from lipid exchange for mechanistic studies of neurotransmitter release. Proc. Natl. Acad. Sci. 108, E304-E313 (2011).
35. Kyoung, M., Zhang, Y., Diao, J., Chu, S. & Brunger, A. T. Studying calcium-triggered vesicle fusion in a single vesicle-vesicle content and lipid-mixing system. Nat. Protoc. 8, 1-16 (2012).
36. Lai, Y. et al. Complexin inhibits spontaneous release and synchronizes Ca²⁺-triggered synaptic vesicle fusion by distinct mechanisms. Elife 3, e03756 (2014).
37. Lai, Y. et al. Molecular Mechanisms of Synaptic Vesicle Priming by Munc13 and Munc18. Neuron 95, 591-607.e10 (2017).
38. Li, H. et al. Cre-mediated recombination in mouse Clara cells. genesis 46, 300-307 (2008).
39. Liu, J. et al. ErbB2 Pathway Activation upon Smad4 Loss Promotes Lung Tumor Growth and Metastasis. Cell Rep. 10, 1599-1613 (2015).
40. Lu, L. et al. Structure-based discovery of Middle East respiratory syndrome coronavirus fusion inhibitor. Nat. Commun. 5, 3067 (2014).
41. Luo, F. & Sudhof, T. C. Synaptotagmin-7-Mediated Asynchronous Release Boosts High-Fidelity Synchronous Transmission at a Central Synapse. Neuron 94, 826-839.e3 (2017).
42. Ma, C., Su, L., Seven, A. B., Xu, Y. & Rizo, J. Reconstitution of the vital functions of Munc18 and Munc13 in neurotransmitter release. Science 339, 421-5 (2013).
43. Ma, C., Li, W., Xu, Y. & Rizo, J. Munc13 mediates the transition from the closed syntaxin-Munc18 complex to the SNARE complex. Nat. Struct. Mol. Biol. 18, 542-9 (2011).
44. Mackler, J. M., Drummon, J. A., Loewen, C. A., Robinson, I. M., and Reist, N. E. Th C(2)B Ca(2+)-binding motif of synaptotagmin is required for synaptic transmission in vivo. Nature. 18, 340-4 (2002)
45. Moiola, M., et al. Stapled Peptids—A Useful Improvement for Peptide-Based Drugs. Molecules 24, 3654-(2019).
46. Pang, Z. P. et al. Synaptotagmin-2 is essential for survival and contributes to Ca²⁺ triggering of neurotransmitter release in central and neuromuscular synapses. J. Neurosci. 26, 13493-504 (2006).
47. Patton, J. S., et al., The particle has landed—characterizing the fate of inhaled pharmaceuticals. J Aerosol Med Pulm DrugDelivery, 23:S71-S87 (2010). Phillips, J. C. et al. Scalable molecular dynamics on CPU and GPU architectures with NAMD. J. Chem. Phys. 153, 044130 (2020).
48. Podbilewicz, B. Virus and Cell Fusion Mechanisms. Annu. Rev. Cell Dev. Biol. 30, 111-139 (2014).
49. Ren, B. et al. SNAP23 is selectively expressed in airway secretory cells and mediates baseline and stimulated mucin secretion. Biosci. Rep. 35, e00220 (2015).
50. Riento, K. et al. Interaction of Munc-18-2 with syntaxin 3 controls the association of apical SNARES in epithelial cells. J Cell Sci. 111, 2681-2688 (1998).
51. Russell, C. J., Jardetzky, T. S. & Lamb, R. A. Membrane fusion machines of paramyxoviruses: Capture of intermediates of fusion. EMBO J. 20, 4024-4034 (2001).
52. Schafmeister, C. E., Po, J. & Verdine, G. L. An All-Hydrocarbon Cross-Linking System for Enhancing the Helicity and Metabolic Stability of Peptides. J. Am. Chem. Soc. 122, 5891-5892 (2000).
53. Speltz, T. E. et al. Stapled Peptides with γ-Methylated Hydrocarbon Chains for the Estrogen Receptor/Coactivator Interaction. Angew. Chemie-Int. Ed. 55, 4252-4255 (2016).
54. Sudhof, T. C. Neurotransmitter Release: The Last Millisecond in the Life of a Synaptic Vesicle. Neuron 80, 675-690 (2013).
55. Sutton, R. B. B., Fasshauer, D., Jahn, R. & Brunger, A. T. Crystal structure of a SNARE complex involved in synaptic exocytosis at 2.4 Aresolution. Nature 395, 347-353 (1998).
56. Thorn, P., Zorec, R., Rettig, J. & Keating, D. J. Exocytosis in non-neuronal cells. J. Neurochem. 137, 849-859 (2016).
57. Turner, J. et al. Goblet Cells Are Derived from a FOXJ1-Expressing Progenitor in a Human Airway Epithelium. Am. J. Respir. Cell Mol. Biol. 44, 276-284 (2011).
58. Tuvim, M. J. et al. Synaptotagmin 2 couples mucin granule exocytosis to Ca²⁺ signaling from endoplasmic reticulum. J. Biol. Chem. 284, 9781-9787 (2009).
59. Watanabe, S. et al. Functional Importance of the Coiled-Coil of the Ebola Virus Glycoprotein. J Virol. 74, 10194-10201 (2000).
60. Wills-Karp, M. et al. Interleukin-13: Central Mediator of Allergic Asthma. Science. 282, 2258-2261 (1998).
61. Winkelmann, V. E. et al. Inflammation-induced upregulation of P2X 4 expression augments mucin secretion in airway epithelia. Am. J. Physiol. Cell. Mol. Physiol. 316, L58-L70 (2019).
62. Xia, S. et al. Fusion mechanism of 2019-nCoV and fusion inhibitors targeting HR1 domain in spike protein. Cell. Mol. Immunol. 17, 765-767 (2020).
63. Yang, X. et al. Syntaxin opening by the MUN domain underlies the function of Munc13 in synaptic-vesicle priming. Nat. Struct. Mol. Biol. 22, 547-554 (2015).
64. Yang, J. T., Wu, C.-S. C. & Martinez, H. M. Calculation of protein conformation from circular dichroism. Methods Enzymol. 130, 208-269 (1986).
65. Zhou, Q. et al. Architecture of the synaptotagmin-SNARE machinery for neuronal exocytosis. Nature 525, 62-67 (2015).
66. Zhou, Q. et al. The primed SNARE-complexin-synaptotagmin complex for neuronal exocytosis. Nature 548, 420-425 (2017).
67. Zhu, Y. et al. Baseline goblet cell mucin secretion in the airways exceeds stimulated secretion over extended time periods, and is sensitive to shear stress and intracellular mucin stores. PLoS One 10, 1-27 (2015).
68. Zhu, Y. et al. Munc13-2−/− baseline secretion defect reveals source of oligomeric mucins in mouse airways. J. Physiol. 586, 1977-92 (2008).

### EXAMPLES

The following examples are provided to illustrate, but not to limit, the claimed invention.

### Example 1. Materials and Methods

Mice. All experiments were approved by the Institutional Animal Care and Use Committee of MD Anderson Cancer Center. Syt2 conditional deletant mice with the second exon flanked by LoxP recombination sites (Syt2^{F/F}) were obtained from Dr. Thomas C. Sudhof (Luo and Sudhof, 2017). These were generated on a mixed 129/Sv:C57BL/6 background and backcrossed by us for ten generations onto a C57BL/6J background. To delete Syt2 in airway epithelial cells, Syt2^{F/F} mice were crossed with mice in which a Cre recombinase optimized for mammalian codon usage was knocked into the secretoglobin 1A1 locus (Scgb1a1^{Cre}) (Liu et al., 2015; Li et al., 2008). Half the progeny from crossing Syt2^{F/F} mice with Syt2^{F/F} mice that were also heterozygous for the Scgb1a1^{cre} allele were Syt2 deletant (Syt2^{D/D}) mice, and the other half were Syt2^{F/F} mice that served as littermate controls for the mucin secretion experiments. Genotyping was performed by PCR using the oligonucleotide primers for Syt2^{WT} and mutant alleles described in Luo and Sudhof (2017). C57BL/6J mice were purchased from the Jackson Laboratory and used as controls to be certain the Syt^{F/F} allele was not hypomorphic in airway epithelium. Since Syt2^{F/F} mice did not differ from Syt2^{WT} at baseline (data not shown; see U.S. Provisional Appl. No. 63/311,001 at EXTENDED DATA FIG. 1) or in the degree of mucous metaplasia and efficiency of stimulated secretion (FIG. 1), they were used as the primary comparator for Syt2^{D/D} mice to minimize environmental and off-target genetic differences. Mice of both sexes were used from age 6 to 26 weeks.

Mucin secretion and airway mucus occlusion in mice. The efficiency of stimulated mucin secretion and the extent of mucus accumulation in the airway lumen of Syt2 mutant mice were measured as described previously (Jaramillo et al, 2019). Briefly, to increase intracellular mucin content (i.e., induce mucous metaplasia), 3 μg IL-13 (BioLegend) in 40 μl PBS was instilled every other day×3 into the posterior pharynx of mice under isoflurane anesthesia to be aspirated during inhalation. Three days after the last instillation, mucin secretion was stimulated by exposing mice for 10 min to an aerosol of 100 mM ATP in 0.9% NaCl, then lungs were harvested 20 min later. Fractional mucin secretion was calculated as the percentage reduction in intracellular mucin content of individual mice after sequential treatment with IL-13 and ATP compared to the group mean mucin content of mice of the same genotype treated only with IL-13. To measure intracellular airway epithelial mucin content, lungs were inflated via the trachea with 10% neutral buffered formalin to 20 cm water pressure for 24 h at 4° C., then embedded in paraffin. A single transverse 5 μm section was taken through the axial bronchus of the left lung between lateral branches 1 and 2, deparaffinized, rehydrated and stained with periodic acid fluorescent Schiff (PAFS) reagent. Images were acquired using an upright microscope (Olympus BX 60) with a ×40 NA 0.75 objective lens, and intracellular mucin was measured around the circumferential section of the axial bronchus using ImagePro (Media Cybernetics). Images were analyzed by investigators blinded to mouse genotype and treatment, and data are presented as the epithelial mucin volume density, signifying the measured volume of mucin overlying a unit area of epithelial baseline lamina.

To measure airway lumenal mucus content, mucous metaplasia was induced as above, then mucin secretion and bronchoconstriction were induced by exposure for 10 min to an aerosol of 150 mM methacholine. Lungs were harvested and fixed by immersion for 48 h at 4° C. to avoid displacement of lumenal mucus and using methanol-based Carnoy's solution (methacarn) for fixation to minimize changes in mucus volume. A single transverse 5 μm section was taken through the axial bronchus of the left lung between lateral branches 1 and 2 and stained with PAFS as above to evaluate intracellular mucin to ensure secretion had been stimulated. Then, six 5 μm sections of the paraffin blocks caudal to the initial section were taken at 500 m intervals and stained with PAFS. Mucus in the lumens of airways was identified manually and the area summed for all twelve sections using ImagePro (Media Cybernetics).

To measure the efficiency of stimulated mucin secretion and the extent of mucus accumulation in the airway lumen of WT mice treated with peptides, the same procedures as those used for analysis of Syt2 mutant mice were followed, except that both outcomes were measured in the same mouse by fixation of the left lung by inflation with formalin to measure mucin secretion and fixation of the right lung by immersion in methacarn to measure mucus accumulation. Stimulation of mucin secretion with a single methacholine aerosol was used for both outcomes. In addition, a MicroSprayer Aerosolizer (Penn-Century) was used for peptide delivery to the airways, secretion was measured in the left axial bronchus at a site 3 mm distal to the site used in the Syt2 mutant mice, and mucus accumulation was measured in the right caudal lobe.

Protein expression and purification. The same constructs and protocols were used to purify cysteine-free Stx1A, SNAP-25A, VAMP2, and Syt1 as described in Lai et al., 2017. The same constructs and protocols were used to purify NSF, and α-SNAP as described in Choi et al., 2018. The protein sample concentrations were measured by UV absorption at 280 nm, aliquots were flash frozen in liquid nitrogen and stored at −80° C.

Stx3. Full-length human Stx3 was expressed in E. coli strain BL-21 (DE3) with an N-terminal, TEV protease-cleavable, hexa-histidine tag. The expression and purification protocols were mostly identical to that of Stx1A. The protein was expressed overnight at 30° C. in 8 l of autoinducing media. Cell pellets from 8 l of culture were suspended in 1× phosphate-buffered saline, 1 mM EDTA, 1 mM PMSF, and 8 EDTA free protease inhibitor tablets (Roche) supplemented with lysozyme and DNAse I (Sigma). The cells were lysed using a sonicator (Fisher Scientific) and an M-110EH microfluidizer (Microfluidics). Inclusion bodies were removed by a 30 min spin at 13,000 RPM in a JA-14 rotor (Beckman Coulter), and the supernatant was centrifuged at 43,000 RPM for 1.5 h in a Ti-45 rotor (Beckman Coulter) to pellet the membrane. Membranes were resuspended in 20 mM HEPES pH 7.5, 300 mM NaCl, 10% glycerol, and centrifuged at 43,000 RPM for 1 h. The pellet was resuspended once more in the same buffer, dodecylmaltoside (Anatrace) was added to 2%, and stirred for 1.5 h at 4° C. The solubilized membrane was centrifuged at 40,000 RPM for 35 min, and the supernatant was loaded onto a 5 ml column of Nickel-NTA agarose (Qiagen). The column was washed with 20 mM HEPES pH 7.5, 20 mM imidazole, 300 mM NaCl, 110 mM octyl glucoside (Anatrace), 10% glycerol, and the protein was eluted with wash buffer supplemented with 450 mM imidazole and 1 M NaCl. The protein fractions were pooled, digested with 110 ug TEV protease, and dialyzed overnight against 20 mM HEPES pH 7.5, 50 mM NaCl, 110 mM OG, 10% glycerol. The fractions were loaded onto a MonoQ 4.6/100 PE column (GE Healthcare) previously equilibrated with dialysis buffer. The protein was eluted with a gradient of 50 mM to 1 M NaCl over 30 column volumes. Protein concentration was measured by absorption at 280 nm and aliquots were flash frozen in liquid nitrogen and stored at −80° C.

SNAP-23. The expression and purification protocols were mostly identical to those of SNAP-25A. Cysteine free SNAP-23, in which all cysteine residues were changed to serine, was expressed in BL21(DE3) cells using auto inducing media from a pGEX vector as an N-terminal GST tag with a thrombin protease cleavage site to remove the tag. Cells from 4.0 l of induced culture were resuspended in 250 ml of buffer (20 mM HEPES pH 7.5, 300 mM NaCl, 4 mM DTT, 10% glycerol) containing 1 mM PMSF and 5 EDTA free protease inhibitor tablets. Cells were lysed by sonication. The lysate was clarified by centrifugation in a Ti45 rotor for 35 min at 40,000 RPM. The supernatant was bound to 10 ml of Glutathione Sepharose beads (GE Healthcare) for 1 h with stirring at 4° C. The beads were harvested by centrifugation, poured into a column, and washed with 100 ml of buffer (20 mM HEPES pH 7.5, 300 mM NaCl, 4 mM DTT, 10% glycerol). 10 μl of 5 mg/ml thrombin was added to the washed beads along with 5 ml of buffer (20 mM HEPES pH 7.5, 300 mM NaCl, 4 mM DTT, 10% glycerol) and the mixture was rocked overnight at 4° C. to remove the GST tag. The cleaved SNAP-23 sample was washed out of the column using buffer (20 mM HEPES pH 7.5, 300 mM NaCl, 4 mM DTT, 10% glycerol) and concentrated to 5 ml. The sample was injected on to a Superdex 200 (16/60) column (GE Healthcare) equilibrated in 20 mM HEPES pH 7.5, 100 mM NaCl, 10% glycerol. Protein containing fractions were combined and concentrated to ˜100 μM SNAP23. The protein concentration was measured by absorption at 280 nm and aliquots were flash frozen in liquid nitrogen and stored at −80° C.

VAMP8. VAMP8 was expressed in E. coli strain BL-21 (DE3) with an N-terminal, TEV protease-cleavable, GST tag. The protein was expressed overnight at 25° C. in 8.0 l of autoinducing media. Cell pellets were suspended in 20 mM HEPES pH 7.5, 300 mM NaCl, 2 mM DTT, 1 mM EDTA, and 8 EDTA free protease inhibitor tablets supplemented with lysozyme and DNAse I. The cells were lysed using a sonicator (Fisher Scientific) and an M-110EH microfluidizer. Cell debris was removed by a 30 min spin at 13,000 RPM in a JA-14 rotor, and the supernatant was centrifuged at 43,000 RPM for 1 h in a Ti-45 rotor to pellet the membrane. The pellet was resuspended in 20 mM HEPES pH 7.5, 300 mM NaCl, 2 mM DTT, 1 mM EDTA and 1.5% DDM and solubilized at 4° C. with stirring for 2.5 h. The solubilized membrane was centrifuged at 43,000 RPM for 35 min, the supernatant was mixed with 5 ml of Glutathione Sepharose 4B and incubated overnight at 4° C. with end-over-end mixing. The beads were washed with 20 CV of 20 mM HEPES pH 7.5, 300 mM NaCl, 2 mM DTT, 1 mM EDTA, 110 mM OG. The protein was cleaved off the column by resuspending the beads in 2 ml of wash buffer supplemented with 2 mM DTT and 110 μg of TEV protease, and incubating at 4° C. for 1 hr. After digest the column was drained and the flow through (containing cleaved VAMP8) was injected on a Superdex 200 10/300 Increase column (GE Healthcare) equilibrated with 20 mM HEPES pH 7.5, 300 mM NaCl, 2 mM DTT, 110 mM OG. Fractions containing protein were pooled, and protein concentration was measured by absorption at 280 nm. Aliquots were flash frozen in liquid nitrogen and stored at −80° C.

Syt2. Syt2 was expressed in BL21(DE3) cells using auto inducing medium from a pGEX vector as an N-terminal GST tag with a thrombin protease cleavage site to remove the tag. Cells from 4 l of induced culture were re-suspended in 200 ml of buffer (20 mM HEPES pH 7.5, 300 mM NaCl, 1 mM EDTA, 2 mM DTT), containing 4 EDTA free protease inhibitor tablets. Cells were lysed by three passes through the Emulsiflex C5 homogenizer (Avestin) at 15000 psi. Unlysed cells and debris were removed by centrifugation in a JA-14 rotor for 10 min at 8000 RPM, the supernatant was centrifuged again in the same rotor for 10 min at 8000 RPM to remove any final debris. The supernatant from the second spin was then centrifuged in a Ti45 rotor for one h at 40,000 RPM to collect the membranes. Membranes were resuspended using a Dounce homogenizer in 100 ml of buffer (20 mM HEPES pH 7.5, 300 mM NaCl, 1 mM EDTA, 2 mM DTT) and n-dodecylmaltoside was added to a final concentration of 2% (w/v) to solubilize the membranes overnight at 4° C. with stirring. The extract was clarified by centrifugation using the Ti45 rotor at 40000 RPM for 35 min. The extract was applied to a 5 ml bed of glutathione Sepharose by stirring at 4° C. for 2 h. The column was washed with buffer (20 mM HEPES pH 7.5, 300 mM NaCl, 1 mM EDTA, 2 mM DTT) containing 110 mM 3-octyl-glucoside and eluted with buffer (20 mM HEPES pH 7.5, 300 mM NaCl, 1 mM EDTA, 2 mM DTT) containing 110 mM 3-octyl-glucoside and 20 mM reduced glutathione. The GST tag was removed by cleavage with 10 μl of 5 mg/ml thrombin for 2 hours and the Syt2 sample was purified on a monoS column equilibrated in 20 mM HEPES pH 7.5, 100 mM NaCl, 110 mM 3-octyl-glucoside, 2 mM DTT (monoS buffer). After washing the column with monoS buffer (20 mM HEPES pH 7.5, 100 mM NaCl, 110 mM 3-octyl-glucoside, 2 mM DTT), the protein was eluted using a linear gradient from 100 mM to 1 M NaCl. Protein containing fractions were combined, the protein concentration was measured by absorption at 280 nm and aliquots were flash frozen in liquid nitrogen and stored at −80° C.

Syt1 C2B and Syt1_QM. The Syt1 C2B domain and the Syt1_QM mutant (R281A, E295A, Y338W, R398A, R399A) were expressed as GST-tagged fusion proteins in E. coli BL21 (DE3) cells at 30° C. overnight. After harvesting the cells by centrifugation, the sample was resuspended in lysis buffer containing 50 mM HEPES-Na, pH 7.5, 300 mM NaCl, 2 mM DTT and EDTA-free protease inhibitor cocktail, and then subjected to sonication and centrifugation. The supernatant was incubated with Glutathione Sepharose beads. The resin was extensively washed with 50 ml of wash buffer I containing 50 mM HEPES-Na, pH 7.5, 300 mM NaCl, and 1 mM DDT, followed by 50 ml of wash buffer II containing 50 mM HEPES-Na, pH 7.5, 300 mM NaCl, 1 mM DTT, and 50 mM CaCl₂). The GST tag was cleaved overnight at 4° C. with PreScission protease (GE Healthcare) in cleavage buffer containing 50 mM HEPES-Na, pH 7.5, 300 mM NaCl, 1 mM DTT, 2 mM EDTA. The cleaved proteins were purified by monoS column and gel filtration on Superdex 75 (GE Healthcare). The protein concentration was measured by absorption at 280 nm and aliquots were flash frozen in liquid nitrogen and stored at −80° C.

Munc13-2*. The Munc13-2* fragment of Munc13-2 (amino acid range 451-1407, that is, including the C1, C2B, and the C-terminally truncated MUN domains, but excluding residues 1326-1343) was cloned into a pFastBac HTB vector with a GST tag and a PreScission cleavage site. The deletion of residues 1326-1343 in this construct prevents aggregation (Li et al., 2011; Ma et al., 2013), and the C-terminal truncation improves solubility. Cells from 8 l of SF9 cell culture were re-suspended in 200 ml re-suspension buffer (RB) (50 mM Tris, pH 8.0, 500 mM NaCl, 1 mM EDTA, 0.5 mM TCEP, 10% glycerol) containing 6 EDTA-free protease inhibitor tablets. The cells were lysed via 3 passes through the Avestin C5 homogenizer at 15000 psi. The lysate was clarified by centrifugation for 35 min at 40,000 RPM in a Ti45 rotor. The supernatant was mixed with 15 ml Glutathione Sepharose beads at 4° C. stirring for 2 h. The beads were washed using an Akta Prime system (GE Healthcare) with 20 ml RB, 90 ml RB+1% triton X-100, then eluted with RB+50 mM reduced glutathione. Peak fractions were pooled and then 100 μl of 10 mg/ml PreScission protease was added and incubated overnight. The cleaved proteins were purified by gel filtration on Superdex 200. The protein concentration was measured by absorption at 280 nm and aliquots were flash frozen in liquid nitrogen and stored at −80° C.

Munc18-2. Munc18-2 (amino acid range 1-594) was cloned into a pFastBac HTB vector with an N-terminal hexa-histidine tag and a TEV cleavage site. Cells from 4.0 l of a SF9 cell culture were re-suspended in 100 ml re-suspension buffer (RB) (20 mM sodium phosphate, pH 8.0, 300 mM NaCl, 2 mM DTT, 10% glycerol with 1 mM PMSF) containing 6 EDTA-free protease inhibitor tablets. The cells were lysed via 3 passes through the Avestin C5 homogenizer at 15000 psi. The lysate was clarified by centrifugation for 35 min at 40,000 RPM in a Ti45 rotor. The supernatant was mixed with 3 ml Ni-NTA beads at 4° C. stirring for 1 h. The beads were washed using an Akta Prime system with 20 ml each of RB, then eluted with RB+300 mM imidazole. Peak fractions were pooled and then 100 μl of 11 mg/ml TEV protease was added. The mixture was dialyzed overnight against 1 l of 500 ml of 20 mM HEPES, pH 7.5, 300 mM NaCl, 2 mM DTT, 10% glycerol. The TEV cleaved protein was injected on a Superdex 200 column. Peak fractions were combined and the protein concentration was measured by UV absorption at 280 nm. Aliquots of 100 μl were flash frozen in liquid N2 and stored at −80° C.

Stapled peptides. All stapled peptides (SP1-SP12, Table 1), the non-stapled peptide P0 (FIG. 2C), as well as peptide chimeras with membrane-penetrating tags or Biotin at the N-terminus, and fluorescent dye Cy3 labels at the C-terminus (SP9-Cy3, Biotin-SP9-Cy3, PEN-SP9-Cy3, PEN-P9-Cy3, TAT-SP9-Cy3 and TAT-P9-Cy3) (Table 3) as well as the labeled control peptide PO-Cy3 were synthesized by New England Peptides (Gardner, USA) with purity higher than 95%. Peptide synthesis was carried out using solid phase peptide synthesis and FMOC chemistry. The peptides were cleaved using trifluoroacetic acid and standard scavengers. The peptides were purified using reverse phase high pressure liquid chromatography. Within the peptides, α,α-distributed non-natural amino acids of olefinic side chains were synthesized (S5-S stereochemistry, bridging 5 amino acids; R8-R stereochemistry, bridging 8 amino acids). The hydrocarbon-staple was made via Grubbs catalyst (Schafmeister et al., 2000). The N-terminii were acetylated and the C-terminii amidated in order to increase their biological activity. For fluorescent labeling experiments, specified peptides were labeled with Cy3 via maleimide reaction with a C-terminal cysteine residue. The biotin-labeled stapled peptide, biotin-SP9-Cy3, was biotinylated at the N-terminus by cross-linking biotin via the carbon spacer 6-aminohexaonic. The C-terminii of selected SP9 peptides were conjugated to Cy3 fluorescent dyes via maleimide reaction chemistry at pH 7.4 and a 1-2 molar ratio of dye to peptide. After conjugation the peptides were purified again and quality control performed by the manufacturer.

For binding of Biotin-SP9-Cy3 to bacterial toxins, C2 and CRM197 were conjugated to streptavidin. Biotin-SP9-Cy3 and streptavidin-conjugated toxins were mixed in a 10:1 ratio at 30° C. for 30 min before adding to cells.

CD spectroscopy. CD spectra were measured with an AVIV stop-flow CD spectropolarimeter at 190 to 250 nm using a cell with a 1 mm path-length. The sample containing 100 mM of synthesized peptides in PBS buffer (137 mM NaCl, 2.7 mM KCl, 10 mM Na2HPO4, and 2 mM KH2PO4, pH 7.4) was measured at 20° C. For the correction of the baseline error, the signal from a blank run with PBS buffer was subtracted from all the experimental spectra. The α-helical content of each peptide was calculated by dividing the mean residue ellipticity [φ]222obs by the reported [φ]222obs for a model helical decapeptide (Yang et al., 1986).

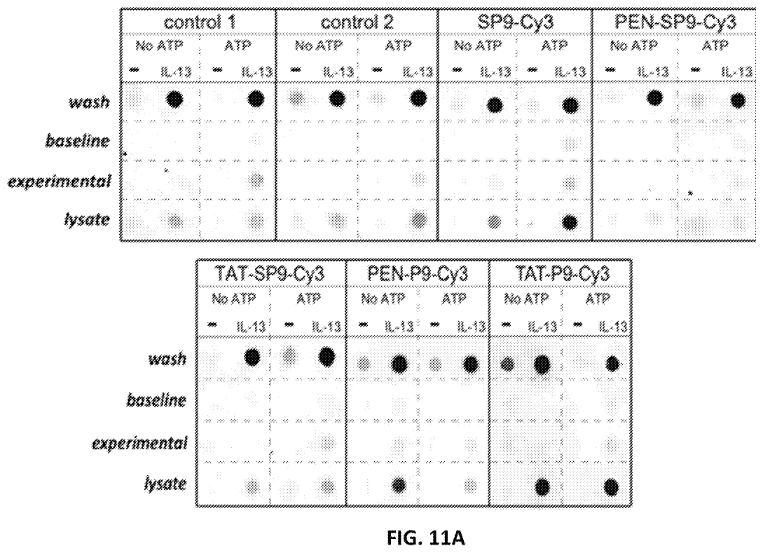

Cryo-electron microscopy. PM and SG vesicles were separately vitrified on lacey carbon grids with a Vitrobot (Thermo Fisher Scientific), and imaged with a FEI Tecnai F20 transmission cryo-electron microscope with a field emission gun (FEI) operated at 200 kV. Images were recorded on a Gatan K2 Summit electron-counting direct detection camera (Gatan) in electron-counting mode (Li et al., 2013). Nominal magnifications of 5,000× and 9,600× (corresponding to pixel sizes of 7.4 Å and 3.8 Å) were used for airway PM and SG vesicles, respectively (FIG. 9B). The diameters of the vesicles (FIG. 9C) were measured with EMAN2 (Tang et al., 2007).

Bulk fluorescence anisotropy measurements. In the bulk fluorescence anisotropy experiments, the P0 and SP9 peptides were labeled with the fluorescent dye Cy3 at the C-terminus. Anisotropy was measured with a Tecan Infinite M1000/PRO fluorimeter using an excitation wavelength of 530±5 nm and emission wavelength of 580±5 nm at 27.2° C. The fluorescent dye labeled samples were diluted to 10 nM concentration in TBS (20 mM Tris, pH 7.5, 150 mM NaCl, 0.5 mM TCEP) for optimal read out.

Molecular dynamics simulations. The starting point for all molecular dynamics simulations was the crystal structure of the SNARE/Syt-1/complexin complex at 1.85 Å resolution (PDB ID 5W5C) (Zhou et al., 2017). Prior to the simulations, the Syt1 C2A domain, the crystallographic water molecules, Mg²⁺, and glycerol molecules were deleted from the crystal structure. Specifically, the following residues that were included in the simulations: Syt1 C2B [270-419], synaptobrevin-2[29-66], Stx1A[191-244], SNAP-25A[10-74 & 141-194]. Cpx1[51-75] was not included in the simulations. For all simulations the NAMD program was used (Phillips et al., 2020). For the primary interface (SNARE: Syt1_C2B) simulations, the Syt1 C2B molecule that produces the primary interface was used. For the simulations with SP9, P9, only residues 37-53 of SNAP-25_N were kept (sequence: EESKDAGIRTLVMLDEQ (SEQ ID NO:10)). The SP9, P9 peptides were simulated with an acetylated N-terminus, and an amidated C-terminus.

The staples for SP9 were created by using CHARMM topology and parameter files for S5 and the covalent bond between S5 residues (Speltz et al., 2016). Initial coordinates were generated by mutating the native residues into Lys using PyMol (Schrödinger, LLC.), and then using the VMD “mutate” command (Humphrey et al., 1996) to change Lys into S5.

For the primary interface (SNARE: Syt1 C2B) simulations, the starting models were placed in a 113×125×116 Å periodic boundary condition box. The empty space in the box was filled with 50,420 water molecules using the VMD solvate plugin. The system has a total of 157,833 atoms. The system was charge-neutralized and ionized by addition of 155 potassium and 138 chloride ions, corresponding to a salt concentration of ˜145 mM using the VMD autoionize plugin.

For the simulations with P9 and SP9, the starting models were placed in a 80×80×80 Å periodic boundary condition box. The empty space in the box was filled with ˜15,200 water molecules using the VMD “solvate” plugin. The system has a total of 48,486 atoms. The system was charge-neutralized and ionized by addition of 42 potassium and 44 chloride ions, corresponding to a salt concentration of ˜145 mM using the VMD “autoionize” plugin.

The CHARMM22 (P9 and SP9:Syt1 C2B simulations) or CHARMM36 (primary interface simulations) all-hydrogen force fields and parameters (Brooks et al., 2009) were used with a non-bonded cutoff of 11 Å. A constant pressure method was used by adjusting the size of the box. The Particle Mesh Ewald method was used to accelerate the calculation of long-range electrostatic nonbonded energy terms. Langevin dynamics (with a friction term and a random force term) was used to maintain the temperature of the simulation. All hydrogen-heavy-atom bonds were kept rigid using the Rattle method as implemented in NAMD.

For the simulations with stapled peptides, in the relaxation step, dihedral angle restraints were added to restrain the S5 CE-CE double bond in the cis conformation, the S5 aliphatic chains in the trans conformation, and all α-helices in the alpha-helical conformation (using the “ssrestraints” plugin for VMD. In all subsequent steps (heating steps, and production runs), all these dihedral angle restraints were turned off. For all other simulations with peptides without staples, in the relaxation step, α-helical (secondary structure) restraints were added for all α-helices (using the ssrestraints plugin for VMD). In all subsequent steps (heating steps, and production runs), all these dihedral angle restraints were turned off. The system was equilibrated by the following procedure: (1) relaxation step, ramping up the temperature from 0 to 50 K for 50 picoseconds with a 1 femtosecond time step; (2) first heating step, ramping up the temperature from 50 to 100 K for 50 picoseconds with a 1 femtosecond time step; (3) second heating step, ramping up the temperature from 100 to 250 K for 150 picoseconds with a 1 femtosecond time step. For all simulations, 1-nanosecond junks were run at a temperature of 300 K with a time step of 1 femtosecond. Five independent 1—μsec simulations were performed for each system (primary interface, SP9-Syt1 C2B, P9-Syt1 C2B) by using different initial random number seeds. All simulations were performed on the Stanford Sherlock Cluster using 4 nodes, each node consisting of dual 10-core CPU 2.4 Ghz Intel processors, i.e. a total of 80 CPUs were used for each simulation. The MPI-parallel NAMD2 2.14b1 executable was used (compiled by Kailu Yang). To visualize the results, only protein components are shown, and all structures were fitted to each other, and displayed with PyMOL.

Vesicle reconstitution. For the ensemble lipid mixing assay, the lipid composition of the SV vesicles was phosphatidylcholine (PC) (46%), phosphatidylethanolamine (PE) (20%), phosphatidylserine (PS) (12%), cholesterol (20%), and 1,1′-dioctadecyl-3,3,3′,3′-tetramethylindodicarbocyanine perchlorate (DiD) (Invitrogen) (2%); for the both neuronal and airway PM vesicles the lipid composition was Brain Total Lipid Extract supplemented 3.5 mol % PIP2, 0.1 mol % biotinylated phosphatidylethanolamine (PE) and 1,1′-dioctadecyl-3,3,3′,3′-tetramethylindocarbocyanine perchlorate (DiI) (Invitrogen). All the lipids are from Avanti Polar Lipids.

For single vesicle content mixing assay, the lipid composition of the SV, SG, VAMP2, or VAMP8 vesicles was phosphatidylcholine (PC) (48%), phosphatidylethanolamine (PE) (20%), phosphatidylserine (PS) (12%), and cholesterol (20%); for both the neuronal and airway PM vesicles the lipid composition was Brain Total Lipid Extract supplemented 3.5 mol % PIP2, and 0.1 mol % biotinylated phosphatidylethanolamine (PE).

The reconstitution method for neuronal PM and SV vesicles was described in detail previously (see Lai et al., 2017; Kyoung et al., 2011; and Lai et al., 2014). The same methods were used for airway PM, SG, VAMP2, and VAMP8 vesicles. Dried lipid films were dissolved in 110 mM OG buffer containing purified proteins at protein-to-lipid ratios of 1:200 for VAMP2 and Stx1A for SV and neuronal PM vesicles, respectively (or 1:200 for VAMP8 and Stx3 for SG and airway PM vesicles, respectively), and 1:800 for Syt1 for SV vesicles (or 1:1200 for Syt2 for SG vesicles).

A 3-5 fold excess of SNAP-25A or SNAP-23 (with respect to Stx1A or Stx3) and 3.5 mol % PIP2 were added to the protein-lipid mixture for neuronal or airway PM vesicles. Detergent-free buffer (20 mM HEPES, pH 7.4, 90 mM NaCl, and 0.1% 2-mercaptoethanol) was added to the protein-lipid mixture until the detergent concentration was at (but not lower than) the critical micelle concentration of 24.4 mM, i.e., vesicles did not yet form. For the preparation of SV, SG, VAMP2, or VAMP8 vesicles for single vesicle content mixing assay, 50 mM sulforhodamine B (Thermo Fisher Scientific) was added to the protein-lipid mixture. The vesicles subsequently formed during size exclusion chromatography using a Sepharose CL-4B column, packed under near constant pressure by gravity with a peristaltic pump (GE Healthcare) in a 5.5 ml column with a ˜5 ml bed volume that was equilibrated with buffer V (20 mM HEPES, pH 7.4, 90 mM NaCl) supplemented with 20 μM EGTA and 0.1% 2-mercaptoethanol. The eluent was subjected to dialysis into 21 of detergent-free buffer V supplemented with 20 μM EGTA, 0.1% 2-mercaptoethanol, 5 g of Bio-beads SM2 (Bio-Rad) and 0.8 g/L Chelex 100 resin (Bio-Rad). After 4 hours, the buffer was exchanged with 21 of fresh buffer V supplemented with 20 μM EGTA, 0.1% 2-mercaptoethanol and Bio-beads, and the dialysis continued for another 12 h (overnight). The chromatography equilibration and elution buffers did not contain sulforhodamine, so the effective sulforhodamine concentration inside SV, SG, VAMP2, or VAMP8 vesicles is considerably (up to ten-fold) lower than 50 mM. For ensemble lipid mixing assay, the reconstitution method is the same as that for the single vesicle content mixing assay, except that 50 mM sulforhodamine B was omitted for all the steps.

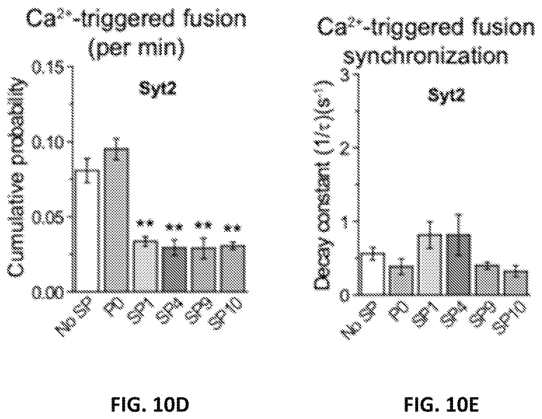

As described previously (Kyoung et al., 2011), the presence and purity of reconstituted proteins in the airway system was confirmed by SDS-PAGE of the vesicle preparations (FIG. 8), and the directionality of the membrane proteins (facing outward) was assessed by chymotrypsin digestion followed by SDS-PAGE gel electrophoresis. The size distributions of the airway PM and SG vesicles were analyzed by electron cryo-microscopy (FIGS. 8B and C), as described previously (Diao et al., 2010).

Bulk lipid mixing experiments with neuronal synaptic proteins. Protein-reconstituted neuronal PM and SV vesicles were mixed at a molar ratio of 1:1, both at 0.1 mM lipid concentration. Lipid mixing is measured by monitoring the fluorescent emission (670 nm) of the DiD dyes in the SV vesicles by FRET using 530 nm laser light excitation of the DiI dyes in neuronal PM vesicles. The fluorescence intensity was monitored in two channels at 570 nm and 670 nm, respectively. Fluorescence changes were recorded with a Varian Cary Eclipse model fluorescence spectrophotometer using a quartz cell of 100 μl with a 5 mm path length. All measurements were performed at 35° C.

Single vesicle content mixing experiments. All single vesicle fusion and single molecule experiments were performed on a prism-type total internal reflection fluorescence (TIRF) microscope using 532 nm (green) laser (CrystaLaser) excitation. Two observation channels were created by a 640 nm single-edge dichroic beamsplitter (FF640-FDi01-25x36, Shemrock): one channel was used for the fluorescence emission intensity of the content dyes and the other channel for that of the Cy5 dye that are part of the injected Ca²⁺-solution. The two channels were recorded on two adjacent rectangular areas (45×90 m²) of a charge-coupled device (CCD) camera (iXon+DV 897E, Andor Technology). The imaging data were recorded with the smCamera software developed by Taekjip Ha, Johns Hopkins University, Baltimore.

Flow chambers were assembled by creating a “sandwich” consisting of a quartz slide and a glass coverslip that were both coated with polyethylene glycol (PEG) molecules consisting 0.1% (w/v) biotinylated-PEG except when stated otherwise, and using double-sided tape to create up to five flow-chambers.

The single vesicle content mixing assay described in Lai et al. (2014) was used to monitor vesicle-vesicle association, Ca²⁺-independent, and Ca²⁺-triggered fusion. The surface of the quartz slides was passivated by coating the surface with polyethylene glycol (PEG) molecules which alleviated non-specific binding of vesicles. The same protocol and quality controls (surface coverage and non-specific binding) were used as described previously (Kyoung et al., 2011; Kyoung et al., 2012) except that PEG-SVA (Laysan Bio) instead of mPEG-SCM (Laysan Bio) was used since it has a longer half-life. The surface was functionalized by inclusion of biotin-PEG (Laysan Bio) during pegylation. A quartz slide was assembled into a flow chamber and incubated with neutravidin for 30 min (0.1 mg/ml).

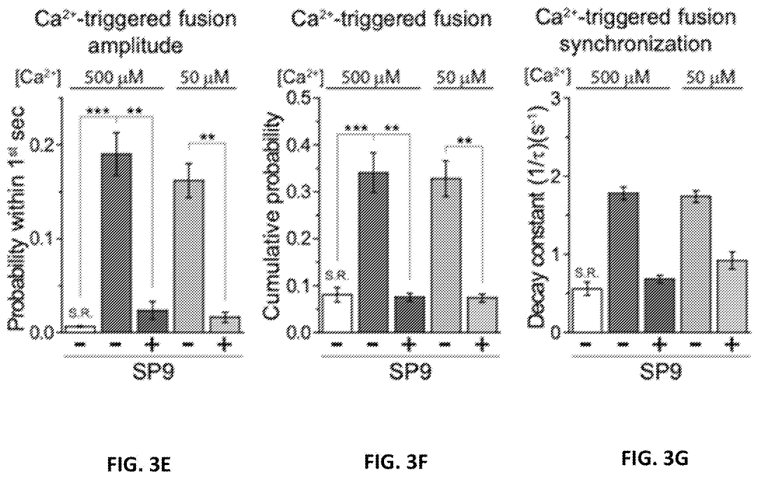

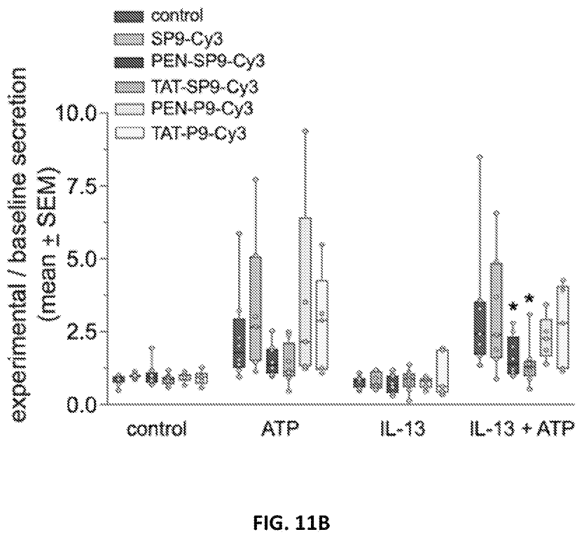

For the fusion experiments described in FIG. 3, and FIGS. 8 and 10, biotinylated neuronal or airway PM vesicles (100× dilution) were tethered to the imaging surface by incubation at room temperature (25° C.) for 30 min followed by three rounds of washing with 120 μl buffer V, in order to remove unbound neuronal or airway PM vesicles; each buffer wash effectively replaces the (3 μl) flow chamber volume more than 100 times. Upon the start of illumination and recording of the fluorescence from a particular field of view of the flow chamber, SV, SG, VAMP2, or VAMP8 vesicles (diluted 100 to 1000 times) were loaded into the flow chamber to directly monitor vesicle association of SG, SV, VAMP2, or VAMP8 vesicles to neuronal or airway PM vesicles for one min; when peptide was included in a particular experiment, it was added concurrently with the SV, VAMP2, or VAMP8 vesicles. While continuing the recording, the flow chamber was washed three times with 120 μl of vesicle buffer (including peptides at the specified concentration, if applicable) in order to remove unbound vesicles. Subsequently, recording was continued for another min to monitor spontaneous fusion events. To initiate Ca²⁺-triggered fusion events within the same field of view, a solution consisting of vesicle buffer, 500 μM Ca²+, 500 nM Cy5 dye molecules (used as an indicator for the arrival of Ca²⁺ in the evanescent field), and, if applicable, peptides was injected into the flow chamber. The injection was performed at a speed of 66 μl/sec by a motorized syringe pump (Harvard Apparatus) using a withdrawal method similar to the one described previously (Kyoung et al., 2012).

Multiple acquisition rounds and repeats for the single vesicle content mixing experiments. In order to increase the throughput of the assay and make better use of the vesicle samples, after intensive washing (3×120 μL) with buffer V (which includes 20 μM EGTA to remove Ca²⁺ from the sample chamber), the entire acquisition sequence (SV, SG, VAMP2, or VAMP8 vesicle loading, counting the number of freshly associated vesicle-vesicle pairs, monitoring of Ca²⁺-independent fusion, Ca²⁺-injection, and monitoring of Ca²⁺-triggered fusion) was repeated in a different imaging area within the same flow chamber. Five such acquisition rounds were performed with the same sample chamber. SV, SG, VAMP2, or VAMP8 vesicles were diluted 1000× for the first and second acquisition rounds, 200× for the third and fourth acquisition rounds, and 100× for the fifth acquisition round in order to offset the slightly increasing saturation of the surface with SG, SV, VAMP2 or VAMP8 vesicles. The entire experiment (each with five acquisition rounds) was then repeated several times (TABLE 4) (referred to as repeat experiment). Among the specified number of repeats there are at least three different protein preps and vesicle reconstitutions, so the variations observed in the bar charts reflect sample variations as well as variations among different flow chambers.

Cell culture. Primary human airway epithelial cells (HAECs) from several donors were obtained from Promocell (Heidelberg, Germany) at passage 2. HAECs from individual donors were thawed and expanded in a T75 flask (Sarstedt) in Airway Epithelial Cell Basal Medium supplemented with Airway Epithelial Cell Growth Medium SupplementPack (both Promocell). Growth medium was replaced every two days. Upon reaching 90 percent confluence, HAECs were detached using DetachKIT (Promocell) and seeded into 6.5 mm Transwell filters (Corning Costar). Filters were precoated with Collagen Solution (StemCell Technologies) overnight and irradiated with UV light for 30 min before cell seeding for collagen crosslinking and sterilization. 3.5×10⁴ cells in 200 μl growth medium were added to the apical side of each filter, and an additional 600 μl of growth medium was added basolaterally. The apical medium was replaced after 48 h. After 72-96 h, when cells reached confluence, the apical medium was removed and basolateral medium was switched to differentiation medium+/−10 ng/ml IL-13 (IL012; Merck Millipore). Differentiation medium consisted of a 50:50 mixture of DMEM-H and LHC Basal (Thermo Fisher) supplemented with Airway Epithelial Cell Growth Medium SupplementPack and was replaced every 2 days. Air-lifting (removal of apical medium) defined day 0 of air-liquid interface (ALI) culture, and cells were grown at ALI conditions until experiments were performed at day 25 to 28. To avoid mucus accumulation on the apical side, HAEC cultures were washed apically with Dulbecco's phosphate buffered solution (DPBS) for 30 min every three days from day 14 onwards.

Mucin secretion assay in HAECs. Mucin secretion experiments under static, i.e., non-perfused, conditions were conducted as detailed previously (Winkelmann et al., 2019; Zhu et al., 2015; Abdullah et al., 2012) with modifications for the peptide treatments. In brief, for the 24 h peptide treatment, 20 μl of DMEM+/−100 μM peptides was added to the apical surface 24 h before stimulation. On the day of the assay, cells were washed 5 times with 100 μl DMEM for 1 h for each wash on the apical side. Apical supernatants were collected after every wash (wash 15), then 100 μl of DMEM+/−100 μM peptides was added to the apical surface and HAECs incubated for 15 min before collecting the supernatant (baseline wash). HAECs were then incubated for an additional 15 min with 100 μl DMEM+/−100 μM ATP (Sigma) before collection of supernatants (experimental washes). Following sample collection, cells were lysed in 100 μl of lysis buffer (lysate) containing 50 mM Tris-HCl pH 7.2, 1 mM EDTA, 1 mM EGTA, 1% Triton-X (Sigma), protease inhibitor cOmplete mini EDTA-free and phosphatase inhibitor PhosSTOP (Roche, Germany). The protocol was adapted for 30 min peptide treatment as follows. After wash 5, 100 μl of DMEM+/−10 μM or 100 μM of peptides was added to the apical surface and HAECs incubated for 30 min (baseline wash). HAECs were then incubated for 30 min with 100 μl DMEM+/−100 μM ATP to collect experimental washes.

All samples were diluted 1:10 in PBS (washes and cell lysates) and 50 μl of each sample were vacuum-aspirated onto a 0.45 μm pore nitrocellulose membrane using the Bio-Dot® MicrofiltrationApparatus (Bio-Rad). Subsequently, membranes were incubated with Intercept blocking buffer (Li-Cor®, USA) for 1 h before probing with anti-MUC5AC (MA1-21907, Invitrogen) added at 1:250 in Intercept blocking buffer for 1 h. Membranes were then washed four times for 10 min in PBS-Tween 20 (PBST) before incubation with the IRDye secondary antibody (#926-33212 or #926-68072; Li-Cor®) for 1 h. All steps were performed at room temperature. Fluorescent signals were acquired using the Odyssey® Fc Imaging System (Li-Cor®, USA) and quantified using ImageJ (v.2.0.0; NIH, USA). Equal volumes of samples were loaded on the gels for control and peptide treatments. Differences in total Muc5ac signal result from differences in IL-13 induced metaplasia between individual filters. Stimulated secretion is therefore normalized to baseline secretion within individual filters to account for filter to filter heterogeneities.

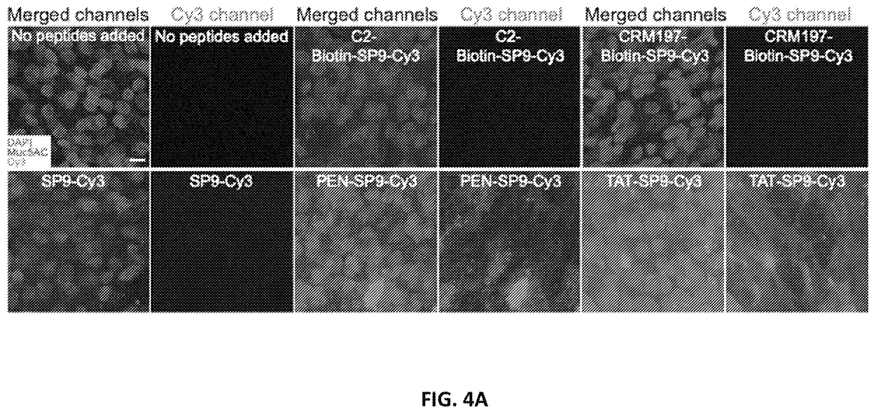

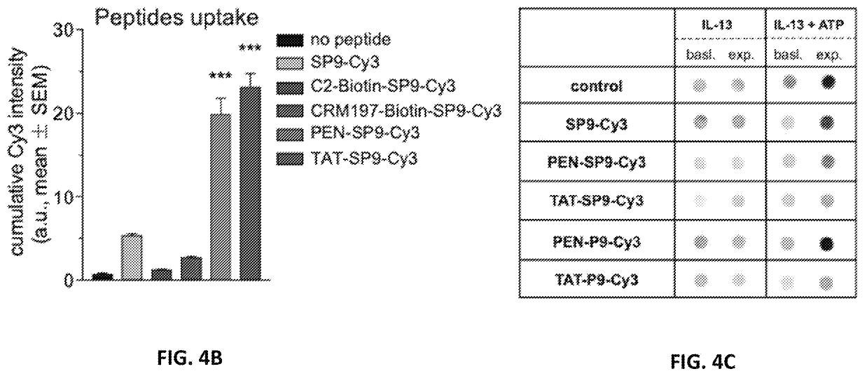

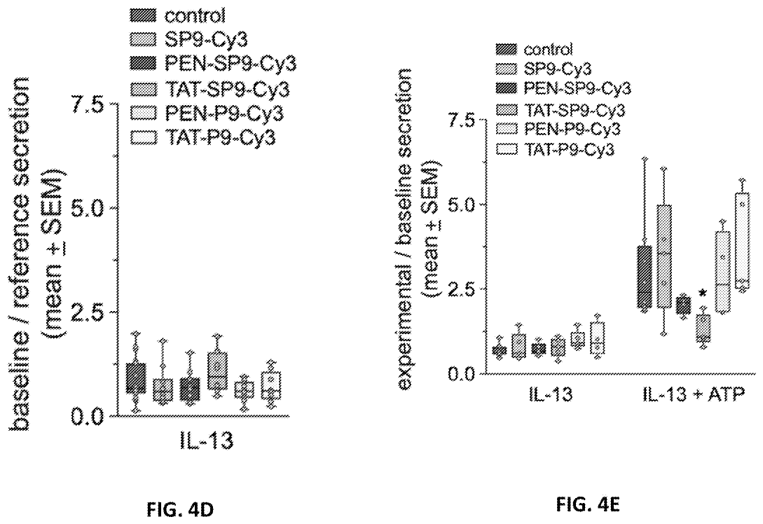

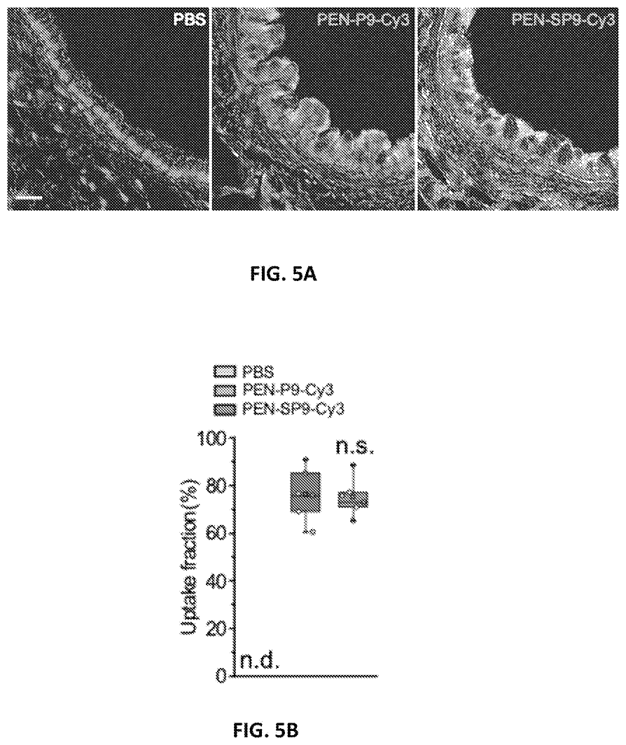

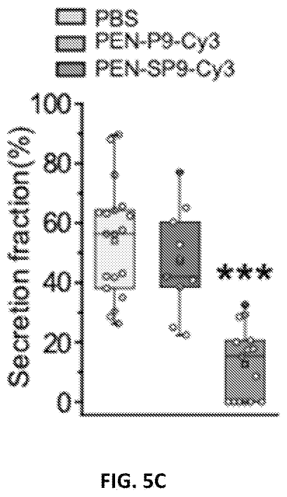

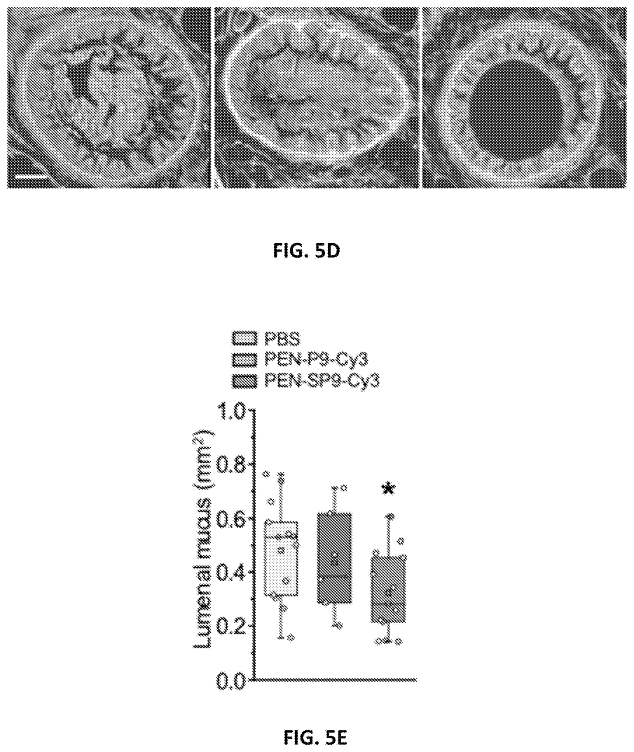

Immunofluorescence staining in HAECs. HAECs grown on Transwell filters were incubated with 20 μl of DMEM+/−100 μM specified peptides on day 28 of establishing ALI. 24 h later, cells were fixed for 20 min in 2% paraformaldehyde in DPBS (data not shown; also see FIG. 4A of U.S. Provisional Patent Application Ser. No. 63/311,001). Cells were then permeabilized for 10 min with 0.2% saponin and 10% FBS (Thermo Scientific) in DPBS. Cells were washed 2 times with DPBS and stained with anti-MUC5AC (clone 45M1; MA1-21907, Thermo Scientific) diluted 1:100 in DPBS, 0.2% saponin and 10% FBS over night at 4° C. Subsequently cells were washed 2 times with DPBS and incubated for 1 h at room temperature in DPBS, 0.2% saponin and 10% FBS containing AlexaFluor 488-labelled anti-mouse secondary antibody (1:500; Thermo Scientific) and DAPI (1: 5000; Thermo Scientific). Images were taken on an inverted confocal microscope (Leica TCS SP5) using a 40× lens (Leica HC PL APO CS2 40×1.30 OIL). Images for the blue (DAPI), green (AlexaFluor 488), red (Cy3) channels were taken in sequential mode using appropriate excitation and emission settings.

Immunofluorescence staining of CALU-3 cells. CALU-3 cells (cells derived from a lung adenocarcinoma patient) were cultivated on a 18-well p-slide (IBIDI) for 2 days. For uptake experiments, cells were left untreated (control), incubated for 30 min with dynasore (80 μM) or chloroquine (100 μM) or put on ice before adding peptides. Cells were incubated for 15, 30, or 60 min with either SP9-Cy3 or PEN-SP9-Cy3, washed, and fixed (15 min 4% PFA in PBS). Cells were counterstained with DAPI. Image acquisition involved acquiring Z-stacks covering the height of the cell (1 m intervals) in 3 different areas per well for each experiment. Z-stacks were collapsed into a single plane and individual cells were identified using “Cellpose 2.0” segmentation algorithm in the nuclear channel. Mean fluorescence intensity in the Cy3 channel was measured for each individual cells identified by the segmentation algorithm.

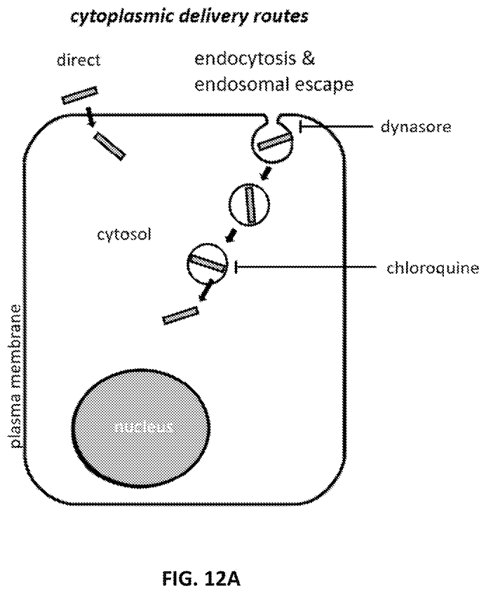

Image analysis for analysis of CPP uptake in HAECs. Serial sections of images along the basolateral to apical cell axis (z-axis) were acquired with 0.28 μm distance between individual z-sections to analyze distribution of intracellular Cy3 fluorescence (FIG. 11). Fluorescent intensity profiles along the z-axis in individual cells were calculated for all channels using the Lecia LAS X software (Leica). In short, fluorescence intensities of DAPI, AlexaFluor 488 (Muc5ac) and Cy3 were analyzed within individual cells at each z-section, normalized and fluorescence intensity traces calculated along the basolateral to apical cell axis. Traces were exported to GraphPad Prism 7 (San Diego, CA, USA) for graph plotting. For quantitative analysis of intracellular Cy3 fluorescence intensities, maximum projections of all z-sections were calculated using the Leica LAS X software and average fluorescence intensities were analyzed for individual Muc5ac⁺ cells.

Quantification and Statistical Analysis. Origin, Matlab, and Prism were used for the generation of all curves and graphs. The fusion experiments were conducted at least three times with different protein preps and vesicle reconstitutions and properties were calculated as mean±SEM. Student's t-test was used to test statistical significance in FIG. 3, and FIGS. 8 and 10 with respect to the specified reference experiment. Statistical significance in FIG. 4 and FIG. 11 was assessed using ANOVA followed by post-hoc Dunnett's test or by two-tailed Student's t-test, where appropriate.

Animal statement. All the mice work was conducted in accordance with the UT MD Anderson Cancer Center IACUC guidelines, and under the IACUC supervision; protocol No 00001214-RN02.

### Example 2. Deletion of Syt2 Protects Mice Against Airway Mucus Occlusion in a Model of Allergic Asthma

Stimulated secretion of mucins hyperproduced in response to inflammation is a major cause of airway obstruction in the pathophysiology of respiratory viral infection, asthma, COPD and cystic fibrosis (Goldblatt et al., 2020; Evans et al., 2015; Bossé et al., 2010; Hays and Fahy, 2003; Boucher et al., 2019). To validate Syt2 as a target of pharmacologic inhibition, whether deletion of Syt2 in airway epithelial cells might protect against mucus occlusion was tested. To accomplish this, Syt2^{F/F} mice (Luo et al., 2017) were crossed with Scgb1a1^{cre} knock-in mice (Liu et al, 2015; Li et al., 2008) as Syt2 knock-out mice die from complications of ataxia by postnatal day 24 (Pang et al., 2006), precluding study of the pathophysiologic role of airway mucin secretion in adult Syt2 knock-out mice. The deletant progeny of this cross (Syt2^{D/D}) were born in a Mendelian ratio and appeared healthy, and the efficiency of deletion was essentially complete (data not shown; also see EXTENDED DATA FIG. 1 of U.S. Provisional Patent Application Ser. No. 63/311,001). There was no spontaneous mucin accumulation in Syt^{D/D} mice that would indicate impairment of baseline mucin secretion (Jaramillo et al., 2018; Zhu et al., 2008). To test impairment of stimulated mucin secretion in these mice, mucous metaplasia was induced by intrapharyngeal instillation of IL-13, then secretion was stimulated with an ATP aerosol. Fractional secretion of intracellular mucin in response to ATP by WT and Syt2^{F/F} mice was 71% and 65%, respectively, whereas it was only 30% in Syt2^{D/D} mice (FIGS. 1A and 1B). To test protection against airway lumenal mucus occlusion, mucous metaplasia was first induced with IL-13, then mucin secretion and bronchoconstriction were stimulated by a methacholine aerosol. The scattered sites of airway mucus occlusion observed in WT and Syt2^{F/F} mice were reduced in Syt2^{D/D} mice, and the cross-sectional area of airway lumenal mucus in a systematic sample of the left lung was reduced in Syt2^{D/D} mice by 74% compared to WT and by 69% compared to Syt2^{F/F} mice (FIGS. 1C and 1D). Normally, the secreted mucin is not visible because it is released into the airway lumen and swept away by beating cilia. Here, the mucus is fixed in place by immersion in fixative rather than lung inflation with fixative, and a bronchoconstrictor is used to minimize clearance.

When bronchial airways of Syt2^{WT} and Syt2^{D/D} mice were stained with antibodies to Syt2 (Abcam #113545, rabbit polyclonal), there was less intense linear staining of tufted ciliated cells in the region of ciliary basal bodies, which often stain non-specifically (data not shown; also see EXTENDED DATA FIG. 1 of U.S. Provisional Patent Application Ser. No. 63/311,001). Secretory cells in Syt2^{WT} mice have a domed appearance with their apical poles staining intensely for Syt2. Secretory cells in Syt2^{D/D} mice did not stain for Syt2, but linear staining of ciliated cells was similar to Syt2^{WT} (data not shown; also see EXTENDED DATA FIG. 1 of U.S. Provisional Patent Application Ser. No. 63/311,001). We enumerated secretory cells in 3 mice of each genotype and found 46% in Syt2^{WT} and 48^{D/D} in Syt2^{D/D}±4%, which did not differ significantly, indicating there was no loss of viability of secretory cells in Syt2^{D/D} mice (data not shown; also see EXTENDED DATA FIG. 1 of U.S. Provisional Patent Application Ser. No. 63/311,001). In Syt2^{WT}mice, 91% of secretory cells stained for Syt2, whereas in Syt2^{D/D}mice, only 7% stained for Syt2, indicating a deletion efficiency ˜92%. Results for Syt2^{F/F}mice were indistinguishable from those for Syt2^{WT} (data not shown). Together these data validated Syt2 as a therapeutic target in muco-obstructive airway disease.

### Example 3. Design and Characterization of Stapled Peptides

Theoretically, helical peptides, such as a fragment of SNAP-25A involved in the primary interface), could be used to selectively interfere with this synaptotagmin-SNARE interaction and thereby disrupt the process of Ca²⁺-triggered membrane fusion (data not shown; also see FIG. 2A and EXTENDED DATA FIG. 2 of U.S. Provisional Patent Application Ser. No. 63/311,001). From a therapeutic perspective, peptide-based strategies have been successfully applied to inhibit virus-host membrane fusion (Xia et al., 2020; Kilby et al., 1998; Russell et al., 2001; Watanabe et al., 2000; Lu et al., 2014). However, the molecular mechanisms are quite different since viral membrane fusion is mediated by formation of a six-helix bundle (Harrison, 2015) whereas Ca²⁺-triggered membrane fusion is mediated by formation of four-helix SNARE bundles in cooperation with synaptotagmins and other factors (Zhou et al., 2015; Sutton et al., 1998). Moreover, peptide-based viral inhibitors function extracellularly whereas peptide-based secretory/synaptic vesicle inhibitors need to act intracellularly. Thus, the question if a peptide inhibitor strategy can be applied to disrupt Ca²⁺-triggered membrane fusion was investigated.

Peptides typically have little secondary structure in solution when taken out of context of the intact system. Thus, their efficacy as in vivo reagents may be limited by their loss of secondary structure. In analogy to the use of stapled peptides to inhibit HIV virus infection (Bird et al., 2010), non-natural amino acids containing olefin-bearing groups were used to generate hydrocarbon-stapled peptides by a Grubbs catalyst (Schafmeister et al., 2000) to interfere with the primary interface. A close-up view of the primary interface indicates the locations of residues that are important for the primary interface include R281, E295, Y338, R398, R399 in Syt1 C2B; which correspond to residues mutated in Syt1_QM) and K40, D51, E52, E55, Q56, D166 in SNAP-25A and D231, E234, E238 in Stx1A (data not shown; also see EXTENDED DATA FIG. 2C of U.S. Provisional Patent Application Ser. No. 63/311,001). The residues directly involved in the primary interface are identical for Syt1/Syt2 and mostly identical for SNAP-25A/SNAP-23, as discussed above in this disclosure (see SEQ ID NOS: 1, 2, 444-47; also see EXTENDED DATA FIGS. 2A and 2B of U.S. Provisional Patent Application Ser. No. 63/311,001). Since the crystal structure is known for the neuronal system, it was first used for the design of stapled peptides that disrupt this interaction, and the peptides in the airway epithelial system were subsequently tested. A series of hydrocarbon-stapled peptides consisting of SNAP-25A fragments that included many of the key residues involved in the primary interface (described previously in Section II; also see FIG. 2A and EXTENDED DATA FIGS. 2A-2C of U.S. Provisional Patent Application Ser. No. 63/311,001)) were designed. Within the peptides, α,α-distributed non-natural amino acids containing varying length of olefinic side chains were synthesized (see Example 1). The hydrocarbon staple was made to flank three (substitution position i and i+4) or six (substitution position i and i+7) amino acids within the SNAP-25A fragments (data not shown; also see FIGS. 2B and 2C of U.S. Provisional Patent Application Ser. No. 63/311,001). The positions of these substitutions were chosen to be away from the primary interface. P0, a non-stapled SNAP-25A fragment, that displays only 5% helicity in solution thus indicating that it is largely a random coil, (FIG. 2A) was used as a control. In contrast, many of the stapled peptides had substantial helical content, and the peptides with two hydrocarbon staples demonstrated better helical stabilization, with helical content up to 86% (FIG. 2A). None of the peptides showed substantial aggregation in size exclusion chromatography, though most of them formed dimers or oligomers (FIG. 6).

To determine whether the stapled peptides specifically interact with the C2B domains of Syt1 and Syt2, the most helical peptide, SP9, and was chosen and labeled with the fluorescent dye Cy3 at the C-terminus and nothing, Biotin, PEN, or TAT at the N-terminus (Table 3). A TAT-P9-Cy3 peptide was used as a control. In Table 3, Ac is acetyl; LCBiot is a biotinylation at the N terminus by cross-linking biotin through the carbon spacer 6-aminohexaonic; and Cy3Mal-amide is Cy3 maleimide, a mono-reactive dye containing maleimide group, which can selectively and efficiently attach Cyanine3 fluorophore.

TABLE 3
Stapled peptide sequences used in immunofluorescence experiments and their N-
and C-terminal modifications

| SEQ | | | | | |
|---|---|---|---|---|---|
| ID NO | Peptide | Linkages | N-term | C-term | Sequence |
| 36 | SP9-Cy3 | [(3, 7; | Ac | (C/Cy3Mal)-amide | Ac- |
| | | 10, 14)] | | | EE(S5)KDA(S5)IR(S5)LVM(S5)DEQ- |
| | | | | | (C/Cy3Mal)-amide |
| 37 | Biotin- | [(3, 7; | LCBiot | (C/Cy3Mal)-amide | LCBiot- |
| | SP9-Cy3 | 10, 14)] | | | EE(S5)KDA(S5)IR(S5)LVM(S5)DEQ- |
| | | | | | (C/Cy3Mal)-amide |
| 38 | PEN- | [(19, 23; | H2N | (C/Cy3Mal)-amide | H2N- |
| | SP9-Cy3 | 26, 30)] | | | RQIKIWFQNRRMKWKKEE(S5)KDA |
| | | | | | (S5)IR(S5)LVM(S5)DEQ-(C/Cy3Mal)- |
| | | | | | amide |
| 39 | PEN-P9- | | H2N | (C/Cy3Mal)-amide | H2N- |
| | Cy3 | | | | RQIKIWFQNRRMKWKKEESKDAGI |
| | | | | | RTL VMLDEQ-(C/Cy3Mal)-amide |
| 40 | TAT- | [(16, 20; | H2N | (C/Cy3Mal)-amide | H2N- |
| | SP9-Cy3 | 23, 27)] | | | GRKKRRQRRRPPQEE(S5)KDA(S5) |
| | | | | | IR(S5)LVM(S5)DEQ-(C/Cy3Mal)- |
| | | | | | amide |
| 41 | TAT-P9- | | H2N | (C/Cy3Mal)-amide | H2N- |
| | Cy3 | | | | GRKKRRQRRRPPQEESKDAGIRTL |
| | | | | | VMLDEQ-(C/Cy3Mal)-amide |
| 52 | PO-Cy3 | | | (C/Cy3Mal)-amide | EESKDAGIRTL VMLDEQGEQLD- |
| | | | | | (C/Cy3Mal)-amide |

Bulk fluorescence anisotropy was recorded after mixing Cy3 labeled SP9 with varying concentrations of Syt1 C2B, Syt2 C2B, or a quintuple mutant of Syt1 C2B (C2B_QM) (SEQ ID NOS: 49-51; also see FIG. 2B of U.S. Provisional Patent Application Ser. No. 63/311,001). SP9 binds to Syt1 C2B and Syt2 C2B with a dissociation constant (Kd) of 24 μM and 35 μM, respectively, which is comparable to the Kd between Syt1 and the SNARE complex (˜20 μM) (Zhou et al., 2015). As a control and as expected, SP9 does not bind to the quintuple mutant of Syt1 C2B_QM since that mutation disrupts the primary interface. Id. Moreover, no binding of the non-stapled peptide PO to the C2B domain of either Syt1 or Syt2 in the conditions of these experiments was observed.

These binding experiments suggest that the stabilization of SP9 peptide by staples is important for binding. To corroborate this finding, the stability of the SP9 peptide interactions with the C2B domain of Syt1 by five 1-μsec molecular dynamics simulations was assessed, starting with a conformation derived from the crystal structure of the primary complex (data not shown; also see EXTENDED DATA FIGS. 2F and 2G of U.S. Provisional Patent Application Ser. No. 63/311,001). The simulations were performed in a periodic box of water molecules, and potassium and chloride ions at physiological concentrations. Both P9 and SP9 changed conformations during these simulations. Four of the five simulations of SP9 adopt binding poses at the end of the 1 sec simulations that would interfere with the formation of the primary interface (FIG. 2C). In contrast, simulations of this peptide without staples (referred to as P9) produced only one binding pose after a 1 μsec simulation that interferes with primary complex formation (FIG. 2C). Moreover, one simulation of P9:Syt1 C2B resulted in a dissociation event (data not shown; also see EXTENDED DATA FIG. 2H of U.S. Provisional Patent Application Ser. No. 63/311,001). Interestingly, the dissociated peptide P9 is highly dynamic, revealing a variety of distorted, partially helical conformations. Presumably, this increased dynamics of the non-stapled P9 peptide resulted in the destabilization of the interactions with Syt1 C2B, producing the rather different binding poses of P9 (FIG. 2C). As control, five 1-μsec molecular dynamics simulations of the primary interface in a solvated environment were carried out. As expected, the primary interface was stable in these simulations (data not shown; also see EXTENDED DATA FIGS. 2D and 2E of U.S. Provisional Patent Application Ser. No. 63/311,001).

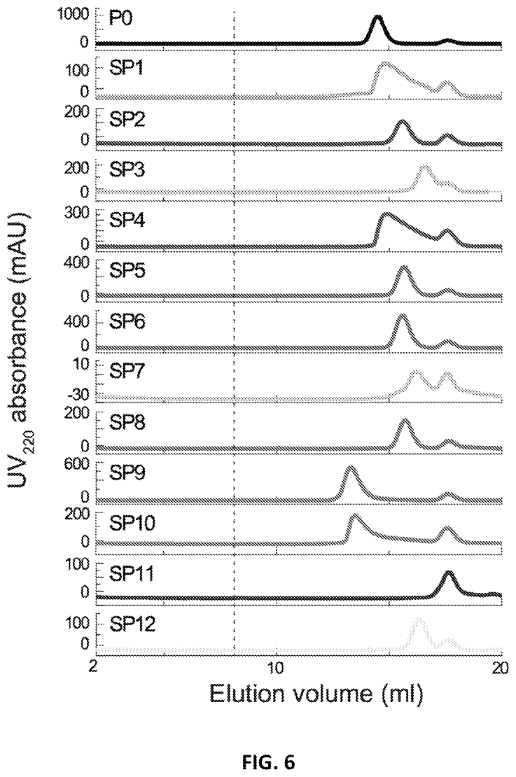

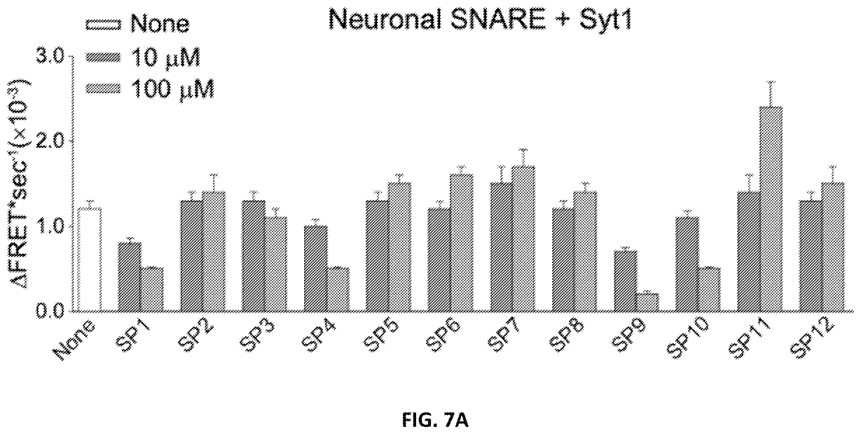

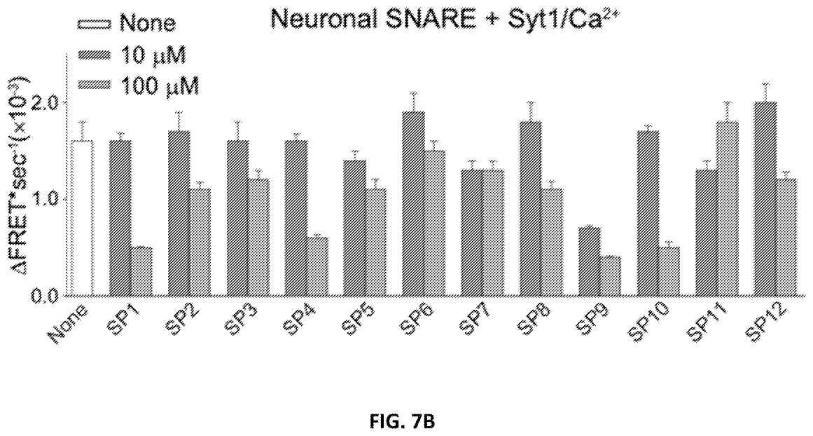

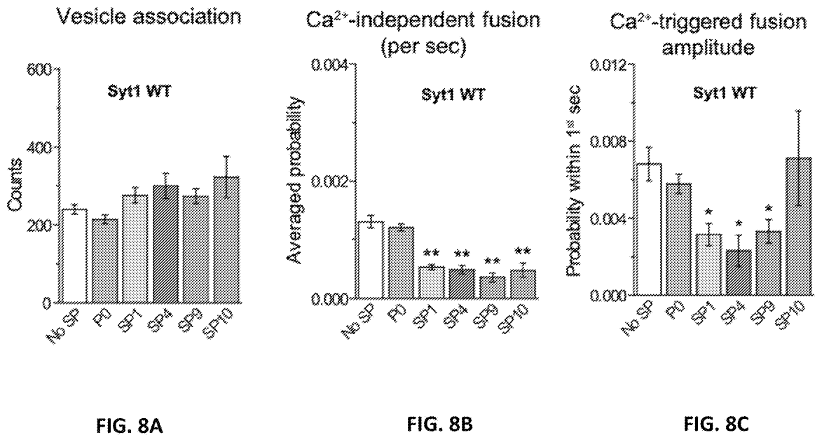

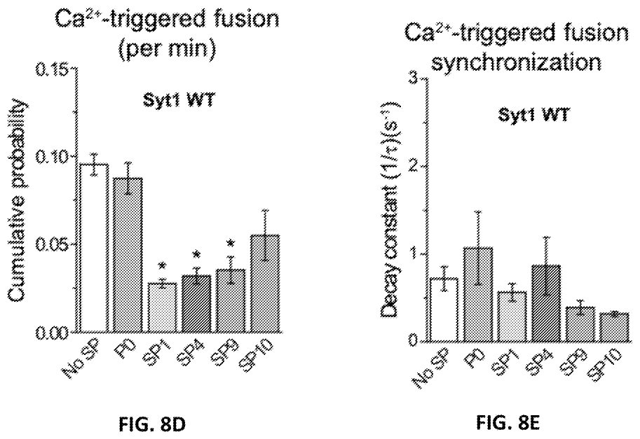

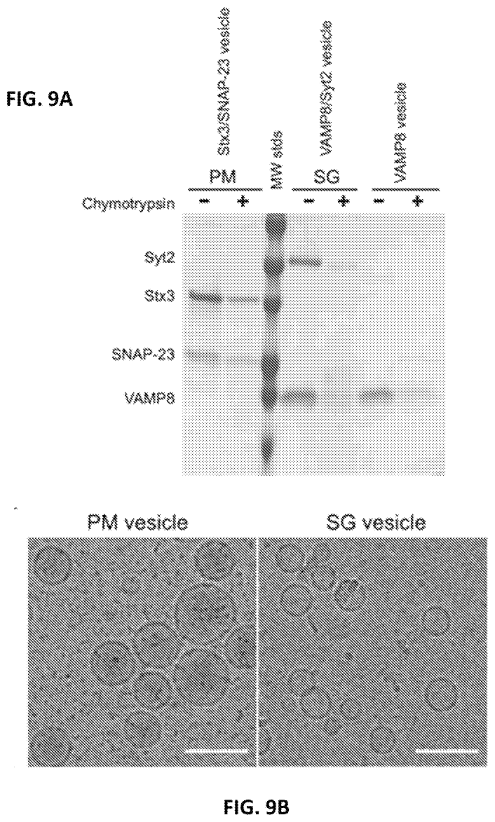

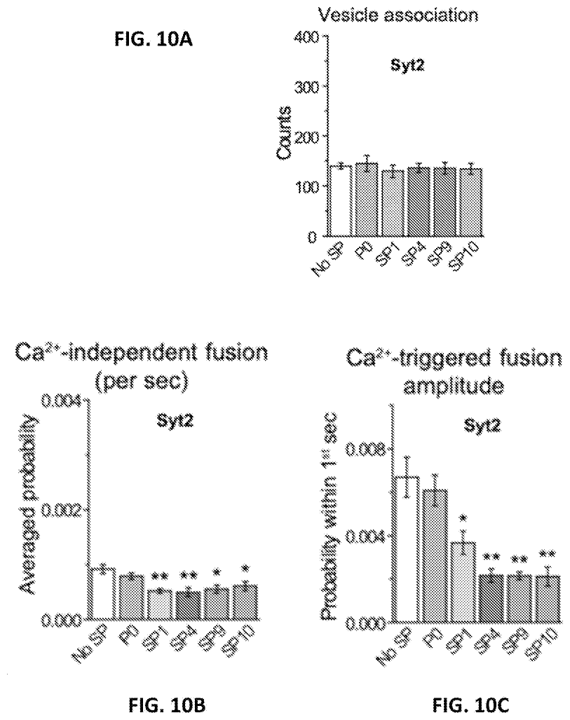

Next, whether the stapled peptides disrupt membrane fusion with reconstituted neuronal SNAREs and nearly full-length Syt1 was tested. A simple lipid mixing ensemble assay (Methods) was used, allowing rapid screening of many peptides. Among the peptides tested, the stapled peptides (SP1, SP4, SP9, SP10) substantially inhibited ensemble lipid mixing in a peptide concentration-dependent fashion with or without Ca²⁺ (FIG. 7A-7B). Notably, the peptides (SP1, SP4, SP9, SP10) that exhibit the largest inhibition in the lipid mixing assay also have the largest helical content (FIG. 2A). The non-stapled peptide (PO) has little effect on Ca²⁺-triggered ensemble lipid mixing measurements of vesicle-vesicle fusion. The two groups of vesicles are mixed at the same molar ratio with a final lipid concentration of 0.1 mM (data not shows; also see EXTENDED DATA FIG. 4C of U.S. Provisional Patent Application Ser. No. 63/311,001). Next, the effects of this subset of stapled peptides were tested in a single vesicle content mixing assay with reconstituted neuronal SNAREs and Syt1 (data not shown; also see EXTENDED DATA FIG. 5A of U.S. Provisional Patent Application Ser. No. 63/311,001) These selected peptides had no effect on vesicle association, but strongly reduced both Ca²⁺-independent and Ca²⁺-triggered full fusion (data not shown; also see EXTENDED DATA FIGS. 5B and 5C-5H of U.S. Provisional Patent Application Ser. No. 63/311,001). As a control, when Syt1 was left out (i.e., vesicles with VAMP2 only, referred to as “VAMP2 vesicles”), or replaced by the quintuple mutant of Syt1 (Syt1_QM) that disrupts binding to the SNARE complex (Zhou et al., 2015), the selected stapled peptides had little effect on either Ca²⁺-independent or Ca²⁺-triggered fusion using the single vesicle content mixing assay (data not shown; also see EXTENDED DATA FIGS. 5I-5R of U.S. Provisional Patent Application Ser. No. 63/311,001). Thus, the inhibitory effect of the stapled peptides observed in this assay is the consequence of interfering with Syt1 binding to the neuronal SNARE complex.

### Example 4. Stapled Peptides Inhibit Vesicle Fusion with Reconstituted Airway Epithelial SNAREs and Syt2

After secretory granule—airway PM vesicle association, vesicle pairs either undergo Ca²⁺-independent fusion or remain associated until fusion is triggered by Ca²⁺ addition. To test if the stapled peptides have an inhibitory effect on the process of mucin secretion, two types of vesicles were reconstituted to mimic mucin secretion: vesicles with reconstituted Stx3 and SNAP-23 that mimic the plasma membrane of epithelial cells (airway PMvesicles), and vesicles with reconstituted VAMP8 and Syt2 that mimic mucin-containing secretory granules (SG vesicles) (Methods) (FIG. 9A-9C). The same subset of the stapled peptides with strong effects in the neuronal system (i.e., SP1, SP4, SP9, SP10) were tested. Inclusion of up to 10 μM of the PO unstapled control peptide in the fusion assay had no effect on the intrinsic Ca²⁺-independent fusion probability or Ca²⁺-triggered cumulative fusion probability (FIG. 10B-10E). In marked contrast, the stapled peptides inhibited the Ca²⁺-independent fusion probability by up to 40% and Ca²⁺-triggered cumulative fusion probability by up to 70% (FIG. 10B-10E). As with the neuronal system, the peptides had no effect on vesicle association (data not shown; also see EXTENDED DATA FIG. 7B of U.S. Provisional Patent Application Ser. No. 63/311,001). As a control, when Syt2 was left out (i.e., vesicles with VAMP8 only, referred to as VAMP8 vesicles), this inhibitory effect of the stapled peptides was eliminated. (data not shown; also see EXTENDED DATA FIGS. 7I-K of U.S. Provisional Patent Application Ser. No. 63/311,001). Together, these results suggest that the selected stapled peptides could inhibit mucin secretion by interfering with interactions between the primary interface of Syt2 and the airway epithelial SNARE complex. Subsequent work focused on SP9 since it strongly affects Ca²⁺-dependent fusion, it has the highest α-helicity, and it is the shortest peptide among all the stapled peptides that were tested. Moreover, as mentioned above, the affinities of SP9 are similar to both Syt2 C2B and Syt1 C2B (FIG. 2B).

### Example 5. SP9 Strongly Inhibits Ca2+-Triggered Vesicle Fusion with Reconstituted Airway Epithelial SNAREs, Syt2, Munc18, and Munc13

TABLE 4 shows a summary of data from the single vesicle fusion experiments as indicated. Among each repeat experiment there are at least three different protein preparations and vesicle reconstitutions, so the variations observed in the bar charts reflect sample variations as well as variations among different flow chambers. For the definition of the repeat experiments see Methods. The effects of 10 μM of each of the specified peptides on the vesicle association, the corresponding Ca²⁺-independent fusion probabilities, the corresponding average probabilities of Ca²⁺-independent fusion events per second, the effects of 10 μM of each of the specified peptides on vesicle association, the corresponding Ca²⁺-independent fusion probabilities, the corresponding average probabilities of Ca²⁺-independent fusion events per second, the corresponding Ca²⁺-triggered fusion probabilities, the corresponding Ca²⁺-triggered fusion amplitude of the first 1-sec time bin upon 500 μM Ca²⁺-injection, the cumulative Ca²⁺-triggered fusion probability within 1 min, and the decay rate (1/T) of the Ca²⁺-triggered fusion histogram were also measured (data not shown; also see EXTENDED DATA FIGS. 5I-5R of U.S. Provisional Patent Application Ser. No. 63/311,001).

As the physiological functions of Munc13 are to catalyze the transition of syntaxin from the syntaxin/Munc18 complex into the ternary SNARE complex (Basu et al., 2005; Ma et al., 2013; Yang et al., 2015) and to promote proper SNARE complex formation (Lai et al., 2017), the effect of the SP9 stapled peptide was tested in a more complete reconstitution that includes airway epithelial SNAREs, Syt2, the C1C2B_MUN2 fragment of Munc13-2 (referred to as Munc13-2*) (Zhu et al., 2008), and Munc18-2 (FIG. 3A) (Kim et al., 2012; Jaramillo et al., 2019). Following previous work (Lai et al., 2017), to form the Stx3/Munc18-2 complex, first the “disassembly factors” (NSF, USNAP, ATP, Mg²⁺) were added along with Munc18-2 to tethered airway PM vesicles. Next, Munc13-2* and SNAP-23 were added, with SG vesicles (data not shown; also see EXTENDED DATA FIG. 3B of U.S. Provisional Patent Application Ser. No. 63/311,001). As a control, in the absence of Munc13-2*, neither SG vesicle tethering nor fusion events were observed because the Stx3/Munc18-2 complex is in the closed conformation and, thus, ternary SNARE complex formation cannot occur (Ma et al., 2011)

TABLE 4

| | | | | |
|---|---|---|---|---|
| Tested peptides or | Ca2+ independent | Ca2+ triggered | Number of analyzed | |
| conditions | fusion events | fusion events | vesicle pairs | Repeats |

neuronal SNAREs and Sytl (related to FIGS. 8A-8E)

| | | | | |
|---|---|---|---|---|
| None | 750 | 846 | 9,557 | 5 |
| P0 | 493 | 591 | 6,543 | 3 |
| SP1 | 235 | 210 | 7,063 | 4 |
| SP4 | 261 | 210 | 8,773 | 4 |
| SP9 | 165 | 272 | 7,122 | 4 |
| SP10 | 263 | 495 | 8,616 | 4 |

neuronal SNAREs only (related to FIGS. 8A-8E)

| | | | |
|---|---|---|---|
| None | 410 | 6,980 | 8 |
| P0 | 104 | 2,020 | 3 |
| SP1 | 245 | 5,650 | 4 |
| SP4 | 356 | 6,246 | 4 |
| SP9 | 336 | 6,546 | 4 |
| SP10 | 341 | 6,198 | 4 |

neuronal SNAREs and Sytl_QM (related to FIGS. 8A-8E)

| | | | | |
|---|---|---|---|---|
| None | 320 | 384 | 6,480 | 3 |
| SP1 | 226 | 282 | 5,044 | 3 |
| SP4 | 222 | 248 | 4,878 | 3 |
| SP9 | 242 | 260 | 2,989 | 3 |
| SP10 | 252 | 367 | 5,329 | 3 |

airway SNAREs and Syt2 (related to FIGS. 10A-10E)

| | | | | |
|---|---|---|---|---|
| None | 461 | 657 | 8,377 | 6 |
| P0 | 113 | 219 | 2,306 | 3 |
| SP1 | 251 | 275 | 8,126 | 4 |
| SP4 | 237 | 231 | 8,017 | 4 |
| SP9 | 274 | 250 | 8,445 | 4 |
| SP10 | 327 | 273 | 8,925 | 4 |

airway SNAREs only (related to FIGS. 10A-10E)

| | | | |
|---|---|---|---|
| None | 54 | 2,578 | 6 |
| P0 | 38 | 1,448 | 3 |
| SP1 | 26 | 1,094 | 4 |
| SP4 | 16 | 660 | 3 |
| SP9 | 22 | 966 | 4 |
| SP10 | 18 | 840 | 3 |

airway system with complete reconstitution (related to FIGS. 3A-3G)

| | | | | |
|---|---|---|---|---|
| No Munc 13-2* | 18 | 30 | 2,751 | 5 |
| Munc 13-2* | 82 | 647 | 1,863 | 4 |
| (500 μM Ca²+) | | | | |
| Munc13-2* + SP9 | 41 | 106 | 1,507 | 3 |
| (500 μM Cat* | | | | |
| Munc13-2* | 95 | 715 | 2,102 | 4 |
| (50 μM Ca²+) | | | | |
| Munc13-2* + SP9 | 23 | 89 | 1,155 | 3 |
| (50 μM Ca21 | | | | |

In contrast, in the presence of Munc13-2*, robust Ca²⁺-triggered fusion was observed at Ca²⁺ concentrations of both 50 and 500 μM (FIGS. 3F-H). When comparing this more complete reconstituted fusion assay with the simpler assay that only uses airway PM vesicles (FIG. 10A-10E), the Ca²⁺-independent fusion probability is similar (“No SP” in FIG. 10Bvs. “No SP9” in FIG. 3E), but the 500 μM Ca²⁺-triggered fusion amplitude, the cumulative fusion probability, and the synchronization are significantly larger (data not shown; also see “No SP” in EXTENDED DATA FIGS. 7E-7H vs. “No SP9” in FIGS. 3F-3I of U.S. Provisional Patent Application Ser. No. 63/311,001). Taken together, the more complete reconstitution that includes Syt2, Munc13-2*, Munc18-2, NSF, and αSNAP improved Ca²⁺-triggered fusion by an order of magnitude compared to the reconstitution with just epithelial airway SNAREs and Syt2. Moreover, the Ca²⁺ sensitivity of the more complete reconstitution is closer to the physiological range (Evans et al., 1999; Kim et al., 1997).

When 10 μM SP9 was added in the more complete reconstitution assay, both the 50 μM and 500 μM Ca²⁻-triggered fusion amplitude, the cumulative fusion probability, and the synchronization were strongly inhibited (FIGS. 3F-3H). The Ca²⁺-independent fusion probability (FIG. 3C) was only moderately reduced compared to the Ca²⁺ triggered amplitude and cumulative fusion probabilities. Moreover, SP9 had no effect on vesicle association (FIG. 3E). Taken together, SP9 specifically inhibits Ca²⁺-triggered membrane fusion in the reconstituted system that includes epithelial airway SNAREs, Syt2, Munc13-2*, Munc18-2, NSF, and αSNAP. From a mechanistic perspective, these results further solidify the critical and active role of the conserved primary (synaptotagmin: SNARE) interface for Ca²⁺-triggered membrane fusion.

### Example 6. SP9 is Efficiently Delivered into Primary Human Airway Epithelial Cells by Conjugation to Cell Penetrating Peptides and Inhibits Stimulated Mucin Secretion

Whether the selected stapled peptides could also inhibit mucin secretion in fully differentiated primary human airway epithelial cells (HAECs) was tested. To facilitate cellular entry of the stapled peptides, the N-terminus of SP9 was conjugated with cell penetrating peptides (CPPs) (Guidotti et al., 2017). Time traces of FRET efficiency upon mixing neuronal PM- and SV-vesicles shows that the non-stapled peptide (PO) had little effect on Ca²⁺-independent ensemble lipid mixing measurements of vesicle-vesicle fusion (data not shown; also see EXTENDED DATA FIG. 4A of U.S. Provisional Patent Application Ser. No. 63/311,001). The two groups of vesicles were mixed at the same molar ratio with a final lipid concentration of 0.1 mM with 10 μM and 100 μM P0 or SP9, respectively. For assessment of cellular entry, these peptides were conjugated with the Cy3 fluorescent dye at the C-terminus. In addition, SP9-Cy3 was conjugated with biotin and bound it to streptavidin-conjugated bacterial toxins (non-toxic mutants of clostridial C2 toxin or diphtheria toxin (CRM197)) as a possible alternative for intracellular delivery. These bacterial toxins can deliver biotin-conjugated peptides into mammalian cells via endocytosis (Fellerman et al., 2020; Fahrer et al., 2013).

Confocal imaging of fixed HAECs treated with SP9-Cy3, conjugated to either bacterial toxins or CPPs, indicated that only CPP-modified SP9-Cy3 penetrated into the cell interior (FIGS. 4A and B). Analysis of intracellular localization in airway secretory cells staining positive for Muc5ac mucin (Muc5ac+ cells) confirmed that CPP-modified SP9-Cy3 was delivered into the cytoplasm of secretory cells, while Biotin-SP9-Cy3 bound to streptavidin-conjugated bacterial toxins was mainly localized to apical mucin granules (data not shown; also see EXTENDED DATA FIG. 8B of U.S. Provisional Patent Application Ser. No. 63/311,001). Quantification of cumulative, intracellular Cy3 fluorescence intensities indicated that both Penetratin (PEN) and TAT efficiently delivered SP9-Cy3 into Muc5ac⁺ epithelial cells (FIGS. 4A and B). Intact epithelial morphology was observed in all conditions (FIG. 4B). Therefore, SP9-Cy3 was conjugated to these CPPs for all subsequent experiments.

### Example 7. SP9 Inhibits Stimulated Mucin Secretion from Airway Epithelial Cells

To investigate the effect of CPP-conjugated SP9-Cy3 on baseline and stimulated secretion under control and mucous metaplastic conditions, HAECs were cultured in the absence or presence of 10 ng/ml IL-13, respectively. IL-13 treatment induces goblet cell hyperplasia and metaplasia in vitro (Turner et al., 2011; Winkelmann et al., 2019) mimicking IL-13 induced mucous metaplasia in vivo (Zhu et al., 2015; Wills-Karp et al., 1998). Consistently, Muc5ac expression was upregulated in IL-13 treated cells (data not shown; also see EXTENDED DATA FIG. 8A of U.S. Provisional Patent Application Ser. No. 63/311,001). Next, experiments were performed in metaplastic conditions with a 30 min peptide pre-incubation at a peptide concentration of 10 μM (data not shown; also see EXTENDED DATA FIG. 4E of U.S. Provisional Patent Application Ser. No. 63/311,001). Baseline secretion was low under metaplastic conditions and not affected by peptide treatment (FIGS. 4C and 4D) and cell morphology appeared normal. Short term treatment with PEN-SP9-Cy3 and TAT-SP9-Cy3 substantially reduced stimulated (i.e., IL-13+ATP) Muc5ac secretion by 48 and 86%, respectively (FIGS. 4C and 4E). As a control and as expected, the unconjugated SP9-Cy3 peptide had no effect on stimulated secretion. The non-stapled P9-Cy3 peptides were also tested (FIG. 4D): 10 μM PEN-P9-Cy3 or TAT-P9-Cy3 had no effect on stimulated secretion, consistent with a specific action of the stapled SP9 on stimulated mucin secretion.

Since the inhibitory effect of TAT-P9-Cy3 was statistically significant while PEN-SP9-Cy3 was just below being statistically significant, the effect of the CPP-conjugated SP9-Cy3 peptides at a higher concentration and longer duration were tested. HAECs were incubated with 100 μM CPP-conjugated SP9-Cy3 for 24 h before stimulation (FIG. 11A). Similar to the experiments at lower peptide concentration, both PEN-SP9-Cy3 and TAT-SP9-Cy3 significantly reduced stimulated Muc5ac secretion by 73% and 83% in metaplastic HAECs, respectively (FIGS. 11A and B). As a further control, SP9-Cy3 (without CPP) had no effect on ATP-stimulated secretion. Taken together, these results suggest that CPP-conjugated SP9-Cy3 inhibits agonist stimulated mucin secretion from secretory airway epithelial cells.

### Example 8. PEN-SP9 Efficiently Enters Mouse Airway Epithelium, Inhibits Stimulated Mucin Secretion, and Attenuates Airway Mucus Occlusion

To investigate whether SP9 could have therapeutic benefit in vivo, SP9 conjugated to CPPs and fluorophores was introduced into mouse airways using a microsprayer inserted into the distal trachea under direct visualization with a laryngoscope. Initial pilot experiments using TAT-SP9-Cy3 and PEN-SP9-Cy3 showed fluorescent labeling of scattered airway epithelial cells, but the forcefully injected peptide solution mostly bypassed the left proximal axial bronchus between lateral branches L1 and L2. All subsequent studies of airway mucin secretion were therefore performed in the distal axial bronchus. Unexpectedly, when TAT-SP9-Cy3 was introduced into the airways of mice with mucous metaplasia, serial sections showed that Cy3-labeled cells had secreted their mucin stores without stimulation by a secretagogue such as ATP or methacholine (data not shown; also see EXTENDED DATA FIGS. 9A and 9B of U.S. Provisional Patent Application Ser. No. 63/311,001). This effect was not observed with the injection of buffer alone. Hence, secretion was not induced by the shear force of the microsprayer, but it appears to be a side-effect of the TAT-SP9-Cy3 compound in this system. Fortunately, PEN-SP9-Cy3 did not show this problem (FIGS. 5A and 5B), so it was used in all subsequent experiments. After stimulation of secretion with methacholine, mice pretreated with PBS or PEN-P9-Cy3 (P9) show greater reductions in intracellular mucin content than mice pretreated with PEN-SP9-Cy3 (data not shown; see also FIG. 5C of of U.S. Provisional Patent Application Ser. No. 63/311,001). Exploratory dose-ranging experiments showed minimal Cy3 labeling below 20 μM PEN-SP9-Cy3, and an apparent plateau above 200 μM, so a concentration 200 μM delivered 30 min before secretagogues was used in all subsequent experiments. Notably, labeling of ciliated cells with PEN-SP9-Cy3 was greater than that of secretory cells, perhaps reflecting the greater apical surface area of ciliated cells (data not shown; also see EXTENDED DATA FIGS. 9C and 9D of U.S. Provisional Patent Application Ser. No. 63/311,001).

The peptide solution was injected into the airway under pressure to generate a droplet aerosol. A high concentration of peptides was used in these experiments in part because the solution is expected to be in contact with the airway epithelium only briefly and to mostly end up in the alveolar region. Serial lung sections after reintroduction of fluorescent labeled peptide confirmed this expectation (data not shown; also see EXTENDED DATA FIG. 9A-9D of U.S. Provisional Patent Application Ser. No. 63/311,001). In serial transverse sections of the left axial bronchus of a mouse with mucous metaplasia induced by prior instillation of IL-13, then treated with 300 μM aerosolized TAT-SP9-Cy3, staining with PAFS demonstrates intracellular mucin, and shows some cells with high mucin content and other cells with low mucin content, presumably due to induced mucin secretion (data not shown; also see EXTENDED DATA FIG. 9A of U.S. Provisional Patent Application Ser. No. 63/311,001). Cells that internalize TAT-SP9-Cy3 tend to have low intracellular mucin content (data not shown; also see EXTENDED DATA FIG. 9A of U.S. Provisional Patent Application Ser. No. 63/311,001).

Sections of the left axial bronchus of mice with mucous metaplasia induced by prior instillation of IL-13subsequently treated with aerosolized 100 mM ATP or 1 mM TAT-SP9-Cy3 show high intracellular mucin content in the mice not treated with an aerosolized drug, extensive secretion of intracellular mucin in the mice treated with ATP, and extensive apocrine mucin secretion in the mice treated with TAT-SP9-Cy3 (data not shown; also see EXTENDED DATA FIG. 9B of U.S. Provisional Patent Application Ser. No. 63/311,001) Transverse section of the left axial bronchus of a mouse taken 30 min after treatment with aerosolized 20 μM PEN-SP9-Cy3 and immunofluorescent staining for CCSP show that secretory cells are observed to not take up PEN-SP9-Cy3, which is presumed to be internalized into ciliated cells, the other major airway epithelial cell type (data not shown; also see EXTENDED DATA FIG. 9C of U.S. Provisional Patent Application Ser. No. 63/311,001). Ciliary tufts were clearly visible by differential interference microscopy, but no staining of intervening secretory cells (data not shown; also see EXTENDED DATA FIG. 9C of U.S. Provisional Patent Application Ser. No. 63/311,001).

Approximate concentrations of PEN-SP9 required for jet nebulizer can be inferred based upon the concentration of PEN-SP9 required to achieve a therapeutic effect in cultured epithelial cells (FIG. 4, 10 μM, rather than 200 μM in mice). Such correlations have been extensively studied, and the concentration of drug in the airway surface liquid can be predicted to be approximately 20% that in the nebulizer (Knust J. et al., 2009; Patton J. S. et al., 2010; Durairaj L., 2004). This assumes an approximate deposition of 0.1% of the nebulized drug per mouse, and an average airway surface liquid volume in mice of 20 μl. These assumptions were tested in previous work with an aerosolized drug (Evans S. E., et al. 2011) and found to be approximately correct (Alfaro V. Y., et al., 2014; Cleaver J. O., et al., 2014).

For allometric scaling for human drug delivery, it is generally believed that concentrations of drug in a jet nebulizer can be kept constant since the lung size of mammals, and hence the inspiratory volume, scales with size. This is certainly true with aerosol drug delivery to human children and adults using the same device and formulation. Consistent with this, here precisely the same concentration of drug in a jet nebulizer in mice administered for a defined period of time that fell on the upper inflection point for a pharmacodynamic marker (Alfaro V Y, et al., 2014) also fell on the upper inflection point of a biomarker in a human dose-escalation study (NCT 02124278). Substantially lower concentrations and/or dosages could be administered if the inhaler used can deliver drug to the conducting airways with high efficiency.

Aerosol administration of 200 μM PEN-SP9-Cy3 labeled 76% of epithelial cells in the distal left axial bronchus, while 200 μM of the control peptide PEN-P9-Cy3 labeled 77% of epithelial cells (FIGS. 5A and 5B). Pretreatment with PEN-SP9-Cy3 markedly reduced the fractional secretion of intracellular mucin stimulated by methacholine (by 82.3%), whereas PEN-P9-Cy3 had no effect (FIG. 5C). Pretreatment with PEN-SP9-Cy3 also significantly reduced airway lumenal mucus accumulation in the right lung (by 33.1%), whereas PEN-P9-Cy3 had no effect (FIGS. 5D and 5E).

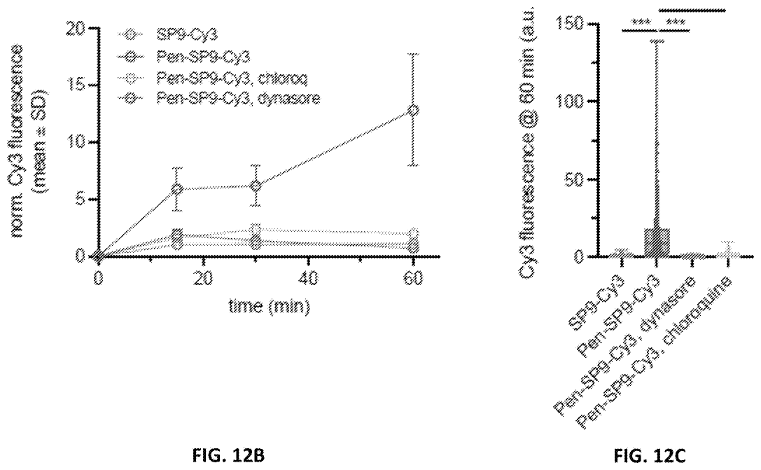

As shown in FIGS. 12A-12C, cytoplasmic delivery of PEN-SP9-Cy3 is inhibited by 80 μM dynasore, an inhibitor of endocytosis, or by endosomal acidification using 100 μM chloroquine. Therefore, cytoplasmic delivery of SP9 depends on endocytosis and endosomal escape.

### Example 9. Sequences of the Disclosure

A listing of the sequences by SEQ ID NO is provided below in Table 5.

TABLE 5
Sequences of disclosure

SEQ ID NO: 1 (P0, SNAP-25A)
EESKDAGIRTLVMLDEQGEQLD
SEQ ID NO: 2 (P0, SNAP-23)
IESQDAGIKTITMLDEQKEQLN
SEQ ID NO: 3 (SP3 with (5, 12)] linkage)
Ac-SKDA(R8)IRGLVM(S5)DEQC-amide
SEQ ID NO: 4 (SP8 with [(5, 9; 12, 16)] linkages)
Ac-SKDA(S5)IRT(S5)VM(S5)DEQ(S5)EQL-amide
SEQ ID NO: 5 (SP1 with [(4, 8; 15, 19)] linkages)
Ac-SKD(S5)GIR(S5)LVMLDE(S5)GEQ(S5)DR-amide
SEQ ID NO: 6 (SP4 with [(5, 9; 12, 16)] linkages)
Ac-SKDA(S5)IRT(S5)VM(S5)DEQ(S5)EQL-amide
SEQ ID NO: 7 (SP7 with [(5, 9; 15, 19)] linkages)
Ac-SKDA(S5)IRT(S5)VMLDE(S5)GEQ(S5)DR-amide
SEQ ID NO: 8 (SP9 with [(3, 7; 10, 14)] linkages)
Ac-EE(S5)KDA(S5)IR(S5)LVM(S5)DEQC
SEQ ID NO: 9 (SP10 with [(4, 8; 11, 15)] linkages)
Ac-SKD(S5)GIR(S5)LV(S5)LDE(S5)GEQL-amide
SEQ ID NO: 10 (P9, SNAP-25A amino acid residues
37-53)
EESKDAGIRTLVMLDEQ
SEQ ID NO: 11 (SP2)
Ac-SKDA(R8)IRTLVM(S5)DEQGEQL-amide
SEQ ID NO: 12 (SP5)
Ac-SKDAGIR(R8)LVMGDE(SS)GEQL-amide
SEQ ID NO: 13 (SP6)
Ac-SKDAGIRT(R8)VMGDEQ(S5)EQL-amide
SEQ ID NO: 14 (SP11)
Ac-SKDA(S5)IRT(S5)V(S5)LDE(S5)GEQL-amide
SEQ ID NO: 15 (SP 12)
Ac-SKD(R8)GIRTLV(S5)LDEQC-amide
SEQ ID NO: 16 (penetratin)
RQIKIWFQNRRMKWKK
SEQ ID NO: 17
VKRGLKLRHVRPRVTRMDV
SEQ ID NO: 18 (HIV glycoprotein 41)
GALFLGFLGAAGSTMGAWSQPKKKRKV
SEQ ID NO: 19 (Tryptophan-rich cluster)
KETWWETWWTEWSQPKKKRKV
SEQ ID NO: 20 (Cadherin)
LLIILRRRIRKQAHAHSK
SEQ ID NO: 21 (p14ARF)
MVRRFLVTLRIRRACGPPRVRV
SEQ ID NO: 22 (Bovine prion)
MVKSKIGSWILVLFVAMWSDVGLCKKRP
SEQ ID NO: 23
KLALKLALKALKAALKLA
SEQ ID NO: 24 (galanin-mastoparan)
GWTLNSAGYLLGKINLKALAALAKKIL
SEQ ID NO: 25 (Azurin)
LSTAADMQGVVTDGMASGLDKDYLKPDD
SEQ ID NO: 26
DPKGDPKGVTVTVTVTVTGKGDPKPD
SEQ ID NO: 27 (Bactenecin family)
RRIRPRPPRLPRPRPRPLPFPRPG
SEQ ID NO: 28 (Antitrypsin)
CSIPPEVKFNKPFVYLI
SEQ ID NO: 29 (C105Y)
PFVYLI
SEQ ID NO: 30 (CHL8)
SDLWEMMMVSLACQY
SEQ ID NO: 31 (MAP)
KLALKLALKALKAALKLA
SEQ ID NO: 32 (Pept1)
PLILLRLLRGQF
SEQ ID NO: 33 (Pept2)
PLIYLRLLRGQF
SEQ ID NO: 34 (IVV-14)
KLWMRWYSPTTRRYG
SEQ ID NO: 35 (Ig(v))
MGLGHLLVLAAALQGAKKKRKV
SEQ ID NO: 36 (SP9-Cy3)
Ac-EE(S5)KDA(S5)IR(S5)LVM(S5)DEQ-(C/Cy3Mal)-
amide
SEQ ID NO: 37 (Biotin-SP9-Cy3)
LCBiot-EE(S5)KDA(S5)IR(S5)LVM(S5)DEQ-(C/Cy3Mal)-
amide
SEQ ID NO: 38 (PEN-SP9-Cy3)
H2N-IRQIKIWFQNRRMKWKKEE(S5)KDA(S5)IR(S5)LVM
(S5)DEQ-(C/Cy3Mal)-amide
SEQ ID NO: 39 (PEN-P9-Cy3)
H2N-RQIKIWFQNRRMKWKKEESKDAGIRTLVMLDEQ-
(C/Cy3Mal)-amide
SEQ ID NO: 40 (TAT-SP9-Cy3)
H2N-GRKKRRQRRRPPQEE(S5)KDA(S5)IR(S5)LVM(S5)DEQ-
(C/Cy3Mal)-amide
SEQ ID NO: 41 (TAT-P9-Cy3)
H2N-GRKKRRQRRRPPQEESKDAGIRTLVMLDEQ-(C/Cy3Mal)-
amide
SEQ ID NO: 42 (Stx1A, aa 222-247)
LVESQGEMIDRIEYNVEHAVDYVERA
SEQ ID NO: 43 (Stx3, aa 222-247)
LVENQGEMLDNIELNVMHTVDHVEKA
SEQ ID NO: 44 (SNAP-25A, aa 156-170)
IIGNLRHMALDMGNE
SEQ ID NO: 45 (SNAP-23, aa 156-170)
ILGNLKDMALNIGNE
SEQ ID NO: 46 (Syt1, aa 269-408)
EEQEKLGDICFSLRYVPTAGKLTVVILEAKNLKKMDVGGLSDPYVKIH
LMQNGKRLKKKKTTIKKNTLNPYYNESFSFEVPFEQIQKVQVVVTVLD
YDKIGKNDAIGKVFVGYNSTGAELRHWSDMLANPRRPIAQWHTL
SEQ ID NO: 47 (Syt2, aa 268-407)
EEPEKLGDICTSLRYVPTAGKLTVCILEAKNLKKMDVGGLSDPYVKIHL
MQNGKRLKKKKTTVKKKTLNPYFNESFSFEIPFEQIQKVQVVVTVLDY
DKLGKNEAIGKIFVGSNATGTELRHWSDMLANPRRPIAQWHSL
SEQ ID NO: 48 (TAT)
GRKKRRQRRRPPQ
SEQ ID NO: 49 (Syt2 C2B, aa 272-422)
EKLGDICTSLRYVPTAGKLTVCILEAKNLKKMDVGGLSDPYVKIHLMQ
NGKRLKKKKTTVKKKTLNPYFNESFSFEIPFEQIQKVQVVVTVLDYDK
LGKNEAIGKIFVGSNATGTELRHWSDMLANPRRPIAQWHSLKPEEEVD
ALLGKNK
SEQ ID NO: 50 (Syt1C2B, aa 271-421)
EKLGDICFSLRYVPTAGKLTVVILEAKNLKKMDVGGLSDPYVKIHLMQ
NGKRLKKKKTTIKKNTLNPYYNESFSFEVPFEQIQKVQVVVTVLDYDK
IGKNDAIGKVFVGYNSTGAELRHWSDMLANPRRPIAQWHTLQVEEEV
DAMLAVKK
SEQ ID NO: 51 (Syt1C2B(QM), aa 271-421)
EKLGDICFSLAYVPTAGKLTVVILAAKNLKKMDVGGLSDPYVKIHLMQ
NGKRLKKKKTTIKKNTLNPAYNESFSFEVPFEQIQKVQVVVTVLDYDK
IGKNDAIGKVFVGYNSTGAELRHWSDMLANPAAPIAQWHTLQVEEEV
DAMLAVKK
SEQ ID NO: 52 (P0-Cy3)
EESKDAGIRTLVMLDEQGEQLD-(C/Cy3Mal)

The foregoing description of some examples has been presented only for the purpose of illustration and description and is not intended to be exhaustive or to limit the disclosure to the precise forms disclosed. It is recognized that those of ordinary skill in the art may make modifications and variations in the embodiments described herein without departing from the spirit or scope of the present disclosure. All publications, patent applications, patents, figures and other references mentioned herein are expressly incorporated by reference in their entirety.

It is noted that the description and/or claims may be drafted to exclude any optional element. As such, this statement is intended to serve as an antecedent basis for use of such exclusive terminology as “solely,” “only,” and the like in connection with the recitation of claim elements or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Where applicable or not specifically disclaimed, any one of the embodiments described herein is contemplated to be able to combine with any other one or more embodiments, even though the embodiments are described, under different aspects of the invention. As such, the preceding general areas of utility are given by way of example only and are not intended to be limiting on the scope of the present disclosure and appended claims. Additional objects and advantages associated with the compounds, compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional advantages, objects, and embodiments are expressly included within the scope of the present invention.

1. A polypeptide comprising a first peptide having at least 64% identity to SEQ ID NO: 1 or SEQ ID NO: 2 attached to a cell penetrating peptide, the peptide comprising a first pair of non-natural amino acids comprising a macrocyclic crosslink.

2. The polypeptide of claim 1, wherein the first peptide comprises a second pair of non-natural amino acids comprising a macrocyclic crosslink.

3. The polypeptide of claim 1, wherein the first pair of non-natural amino acids flanks three or six contiguous amino acid residues in the peptide.

4. The polypeptide of claim 2, wherein the first pair of non-natural amino acids and second pair of non-natural amino acids each flank three contiguous amino acid residues in the peptide, and wherein the three contiguous amino acid residues flanked by the first pair of non-natural amino acids are different than the three contiguous amino acid residues flanked by the second pair of non-natural amino acids.

5. The polypeptide of claim 1, wherein the first pair of non-natural amino acids correspond to amino acids 6 and 13, 7 and 14, 10 and 17, or 11 and 18 in SEQ ID NO: 1 or SEQ ID NO: 2.

6. The polypeptide of claim 1, wherein the first pair of non-natural amino acids correspond to amino acids 3 and 7, 6 and 10, or 7 and 11 in SEQ ID NO: 1 or SEQ ID NO: 2.

7. The polypeptide of claim 1, wherein the second pair of non-natural amino acids correspond to amino acids 10 and 14, 13 and 17, 14 and 18, or 17 and 21 in SEQ ID NO: 1 or SEQ ID NO: 2.

8. The polypeptide of claim 1, wherein the macrocyclic crosslink is positioned on a non-binding side of the first peptide, wherein the non-binding side of the first peptide does not contain amino acid residues that interact with amino acid residues in the C2B domain of a synaptotagmin protein.

9. The polypeptide of claim 1, wherein the first peptide comprises any one of SEQ ID NO: 3 or 4.

10. The polypeptide of claim 1, wherein the first peptide comprises any one of SEQ ID NO: 5, 6, 7, 8, or 9.

11. The polypeptide of claim 10, comprising SEQ ID NO: 8.

12. The polypeptide of claim 1, wherein the cell penetrating peptide has a sequence that is at least 70% identical to at least one cell penetrating peptide listed in Table 1.

13. The polypeptide of claim 1, wherein the cell penetrating peptide comprises penetratin.

14. The polypeptide of claim 1, wherein the cell penetrating peptide is not an HIV-1 TAT peptide.

15. The polypeptide of claim 1, wherein the C-terminus of the cell penetrating peptide is linked to the N-terminus of the peptide.

16. A polynucleotide that inhibits expression of SYT2, wherein the polynucleotide is a SYT2 targeting siRNA, shRNA, ASO, miRNA, or CRISPR/Cas system guide RNA.

17. (canceled)

18. A pharmaceutical composition comprising a polypeptide according to a claim 1, and a pharmaceutically acceptable excipient.

19. (canceled)

20. A method of treating a subject having mucus hypersecretion-based airway obstruction, the method comprising administering to the subject a therapeutically effective amount of the polypeptide of claim 1.

21. The method of claim 20, wherein the subject has a respiratory viral infection, asthma, chronic obstructive pulmonary disease (COPD), or cystic fibrosis.

22. The method of claim 20, wherein the subject has mucus occlusions.

23. The method of claim 20, wherein the peptide is administered to the subject via inhalation.

24. The method of claim 20, further comprising administering a therapeutically effective amount of an inhibitor of at least one of Munc18, VAMP8, Munc13, or Stx3.

25. A method of inhibiting mucin secretion in an airway epithelial cell, the method comprising contacting the airway epithelial cell or a cell derived from an airway epithelial cell with a polypeptide according to claim 1.

26. A method of inhibiting Syt2-mediated stimulated mucin secretion, triggered by Ca²⁺ release after ATP or methacholine bind to hepta-helical PM receptors coupled to Gq, the method comprising contacting an epithelial cell with a polypeptide according to claim 1.
